(19) United States
(12) Patent Application Publication
(10) Pub. No.: US 2023/0313208 A1
(43) Pub. Date: Oct. 5, 2023

(54) RECOMBINANT MICROORGANISMS

(71) Applicants: Academia Sinica, Nankang, Taipei (TW); The Regents of the University of California, Oakland, CA (US)

(72) Inventors: Yu-Hsiao Chen, Nankang (TW); Hsin-Wei Jung, Nankang (TW); Chao-Yin Tsuei, Nankang (TW); James C. Liao, Nankang (TW)

(21) Appl. No.: 18/016,432

(22) PCT Filed: Jul. 14, 2021
(86) PCT No.: PCT/US2021/041540

§ 371 Date: Jan. 16, 2023

Related U.S. Application Data
(60) Provisional application No. 63/051,672, filed on Jul. 14, 2020.

(51) Int. Cl. C12N 15/70; C12N 9/04; C12N 9/88; C12N 9/92; C12N 1/20; C12N 1/32; C12P 7/06; C12P 7/16; C12P 7/46

(52) U.S. Cl. CPC C12N 15/70; C12N 1/205; C12N 1/32; C12N 9/0006; C12N 9/88; C12N 9/92; C12P 7/065; C12P 7/16; C12P 7/46; C12R 2001/19; C12Y 101/01244; C12Y 401/02043; C12Y 503/01006; C12Y 503/01009; C12Y 503/01027

(57) ABSTRACT

Provided herein are metabolically-modified microorganisms that can grow on an organic C1 carbon source.

### CROSS REFERENCE TO RELATED APPLICATIONS

This application claims priority to U.S. Provisional Application Serial No. 63/051,672, filed Jul. 14, 2020, the disclosures of which are incorporated herein by reference.

### TECHNICAL FIELD

Metabolically-modified microorganisms and methods of producing such organisms are provided.

### INCORPORATION BY REFERENCE OF SEQUENCE LISTING

Accompanying this filing is a Sequence Listing entitled “Sequence-Listing_ST25.txt”, created on Jul. 14, 2021 and having 110,913 bytes of data, machine formatted on IBM-PC, MS-Windows operating system. The sequence listing is hereby incorporated herein by reference in its entirety for all purposes.

### BACKGROUND

Methanol, being electron-rich and derivable from methane or CO₂, is a potentially renewable one-carbon (C1) feedstock for microorganisms. Although the ribulose monophosphate (RuMP) cycle used by methylotrophs to assimilate methanol differs from the typical sugar metabolism by only three enzymes, turning a non-methylotrophic organism to a synthetic methylotroph that grows to a high cell density has been challenging.

### SUMMARY

The disclosure provides a synthetic methylotroph (SM) that grows on methanol as the sole carbon source, has a doubling time (t_{D}) of about 12 hours or less. In another embodiment, the SM has a methanol tolerance of ~1.2 M (e.g., from about 50 mM to about 1.2 M). In one embodiment, the SM expresses a polypeptide having methanol dehydrogenase activity, a polypeptide having hexulose-6-phosphate synthase activity, a polypeptide having 3-hexulose-6-phosphate isomerase (sometimes refered to as 6-phospho-3-hexuloisomerase) activity and comprises increased activity of a polypeptide having phosphoglucoisomerase activity, wherein the SM can grow on methanol up to ~1.2 M (e.g., 50 mM, 60 mM, 70 mM, 80 mM, 90 mM, 1 M, 1.1 M, 1.2 M, 1.3 M, 1.4 M or a value between any two of the foregoing values). In another or further embodiment, the SM contains a deletion or reduction in the expression or activity of a glyceraldehyde dehydrogenase A polypeptide, S-(hydroxymethyl) glutathione dehydrogenase A polypeptide, phosphofructokinase polypeptide, histidine-containing protein, and/or a proQ polypeptide. In yet another or further embodiment, the SM has an increased in copy number variation of 2 to 85 of a region between yggE to yghO, rrsA to rrlB, and/or ygiG to smf. In still yet another or further rembodiment, the SM is obtained by engineering a parental microorganism selected from the group consisting of Escherichia, Bacillus, Clostridium, Enterobacter, Klebsiella, Enterobacteria, Mannheimia, Pseudomonas, Acinetobacter, Shewanella, Ralstonia, Geobacter, Zymomonas, Acetobacter, Geobacillus, Lactococcus, Streptococcus, Lactobacillus, Corynebacterium, Streptomyces, Propionibacterium, Synechocystis, Synechococcus, Cyanobacteria, Chlorobi, Deinococcus and Saccharomyces sp. In a further embodiment, the parental microorganism is E. coli. In yet another or further embodiment, the SM further expresses a ribose-5-phosphate isomerase A. In another embodiment, the SM comprises the genetic make up of ATCC deposit accession number.

The disclosure provides a synthetic methylotroph designated Escherichia Coli SM1 having ATCC accession no. PTA-126783. The disclosure further provides progeny and cultures of the microorganism having accession no. PTA-126783.

The disclosure provides a method for producing a metabolite, comprising growing a SM of any of the foregoing embodiments in a medium comprising methanol, whereby the metabolite is produced. In a further embodiment, the metabolite is selected from the group consisting of 4-carbon chemicals, diacids, 3-carbon chemicals, higher carboxylic acids, alcohols of higher carboxylic acids, carotenoids, isoprenoids, cannabinoids and polyhydroxyalkanoates.

The disclosure provides a recombinant microorganism that assimilates a C1 carbon source and comprises a plurality of enzymes selected from the group consisting of Medh, Hps, Phi, Pgi, RpiA, Tkt, Tal and any combination thereof. In one embodiment, the microorganism is obtained by engineering a parental microorganism of the species E. coli. In a further embodiment, the recombinant microorganism comprises a reduction or knockout of a gene selected from the group consisting of pfkA, gapA, frmA, ptsH, proQ and any combination thereof. In a further embodiment, of any of the foregoing, the recombinant microorganism comprises an increased copy number of a region of the genome.

The disclosure provides a recombinant microorganism that expresses one or more heterologous polynucleotide or over-expression of one or more heterologous polynucleotide encoding a polypeptide having methanol dehydrogenase activity, hexulose-6-phosphate synthase activity, 6-phospho-3-hexulose isomerase activity, glucose phosphate isomerase activity and ribose-phosphate isomerase A activity, with a concomitant reduction or elimination of glyceraldehyde-3-phsophate dehydrogenase activity, reduction or elimination of S-(hydroxymethyl)glutathione dehydrogenase (FrmA) activity, reduction or deletion of phosphocarrier protein HPr (also referred to as Histidine-containing protein, HPr and/or PtsH) activity, and the reduction or elimination of ProQ provides, wherein the microorganism grows on methanol.

The disclosure also provides a recombinant microorganism that grows on methanol and comprises the metabolic pathway of FIG. 1A.

The details of one or more embodiments of the disclosure are set forth in the accompanying drawings and the description below. Other features, objects, and advantages will be apparent from the description and drawings, and from the claims.

### BRIEF DESCRIPTION OF THE DRAWINGS

The accompanying drawings, which are incorporated into and constitute a part of this specification, illustrate one or more embodiments of the disclosure and, together with the detailed description, serve to explain the principles and implementations of the invention.

FIGS. 1A-B presents the build and evolution a synthetic methylotrophic E. coli strain. (A) Pathway and mutations relevant to synthetic methylotrophic E. coli SM1. The cog icons represent rationally designed and engineered gene modifications. Solid boxes indicate up-regulated or high copy number genes, while dashed boxes represent genes knocked out or mutated. (B) Flowchart for construction and evolution of synthetic methylotroph. Abbreviations are defined in Table 1. See also FIG. 8 and Table 2.

FIG. 2. Ensemble-Modelling Robust Analysis (EMRA) of the claimed pathway. The x-axis represents the fold change of a specific enzyme activity, while the y-axis refers to the ratio of the 100 parameter sets that are robust at the specific perturbed enzyme activity. Results indicate that high level expression of pfk, gapA, pgk, gpmM, eno, and pyk may cause a system stability problem because of kinetic traps. This result indicates that that high activities of Pfk and enzymes in the lower glycolysis may be detrimental to the system. Hypothesizing that E. coli possesses high glycolytic activity natively, pfkA was knocked out, and knocked down the gapA gene, which is the first gene in the lower glycolysis that is unstable, by replacing it with a functional BL21 gapC gene.

FIGS. 3A-E. Evolution results and verification of E. coli growing on methanol as the sole carbon source. (A) The evolution trajectory (step iv in FIG. 1B) of CFC526.1-20. The media consisted of a decreasing portion of an amino acid mixture (HDA) in MOPS, while keeping methanol at 400 mM. The last passage (purple line) was in methanol only (step v in FIG. 1B). The thick solid line represents HDA percentage in the media. Other lines represent growth curve of cultures in different media (B) Growth curves of CFC 680.1-20 throughout evolution in methanol MOPS (MM) media with nitrate. (C) Growth curve that shows the evolution process of CFC688.1-20 culture with serial inoculation in MM without nitrate. (D) and (E) ¹³C labelling patterns of acetate and formate from CFC680.8. The red lines represent the sample, while the black line illustrates a 13C standard. See also FIG. 9.

FIGS. 4A-D shows DNA-protein crosslinking (DPC) products identified in methylotrophic E. coli cultures. (A) Extended lag phase seen when E. coli is subcultured in methanol media MM at stationary phase. CFC526.40 was being passed from time point I~VI and showed various levels of time lag. Note that starting from time point V, the strain experienced a serious lag phase for growth in methanol. (B) Flow cytometry-based cell viability test. All cells are stained with SYTO-9, while propidium iodide (PI) only stains dead cells when the cell membrane can be penetrated. The coordinates were defined by control samples, including healthy E. coli cells and ethanol-treated dead cells. (C) TEM images of DPC products extracted from different growth stages of CFC526.41, and their uncrosslinked forms. (D) Quantitative proteomics analysis of the proteins from uncrosslinked DPC samples from CFC 526.41 and CFC680.24. Among 6 samples, CFC526.41#2, CFC680.24 #2 and CFC680.24 #3 were selected for analysis based on their similar growth trends. 30 out of 61 common top hits ranked by average abundance were presented. See also FIGS. 10 and 11.

FIGS. 5A-D. Genomic analysis of Methylotrophic E. coli. (A) Venn diagram of mutations of CFC526 along the laboratory evolution process. Single nucleotide variations (SNVs) that are higher than 30% are reported in the graph. The notation 7k, 70k, 130k, and 240k refer to a region spanning the respective size with high copy numbers. The superscripted numbers refer to the type of mutations. (B) Genome structure of SM1. The top part shows Illumina Hiseq mapping coverage of SM1, while the bottom presents a 70k-tandem repeat in SM1 derived from Pacbio and Nanopore sequencing. Some important metabolic genes including a synthetic operon encoding RuMP cycle genes are illustrated. (C) Genome structure of BB1. The 7k region including the ddp operon shows about 84-fold increase in read coverage from the Hiseq mapping. (D) Schematics of the original designed plasmid pFC139 with a rpiAB library, and mutated plasmid pFC139A, B, C emerged during the evolution. See also FIGS. 12, 14, and Table 2.

FIGS. 6A-E shows copy number and plasmid variation in methylotrophic E. coli. (A) Copy number of the multiplicated 70k gene of cultures throughout the evolution process, derived from Illumina Miseq/ Hiseq coverage data. (B) Estimated plasmid composition variation in evolved cultures. The plasmids are categorized into the following: pFC139A, pFC139B, and pFC139C, and the rest of the original pFC139 with RBS library. (C) 70k region copy number dynamics experiment. SM1 was first passed in LB 4 times and MM 1 time subsequently, and then streaked out on a LB plate twice. 7 colonies were then picked and were regarded as individual biological repeats. The colonies were then once again inoculated into LB and was recorded as “Gen1”. They were then passed 3 more times in LB to “Gen4” and another 3 times to “Gen7”. “Gen1”, “Gen4”, and 566 “Gen7” were then inoculated into MM to calculate growth rates. The copy number of the 70k region in LB was determined by digital PCR. The error bars of the copy number are calculated from the mean and SD from sampling 4 genes in the 70k region. The statistical significance between Gen1, Gen4, Gen7 was determined by a t-test, n=4. **p<0.01, *p<0.1, ns= no significance. (D) 70k region copy number comparison between LB cultures and their subsequent MM culture. n=7 (E) 2d-box plot overlaid with scatter plot. The box plot values were calculated by doubling time in methanol and average values of copy numbers. The error bar on the scattered dots are calculated from the mean and SD from sampling 4 genes in the 70k region. n=7.

FIGS. 7A-G shows Characterization of SM1 strain. (A) Core methanol production/ consumption gene transcript ratios (OD600 1.1/0.7) in 400 mM methanol MOPS medium measuring by RNA-seq and qRT-PCR. The RNA-seq results of the ED pathway genes are also shown in dotted bars. (B) The volcano plot of RNA-seq (log2 transcript ratio of OD600 1.1/ 0.7, 400 mM methanol). Triangle: **p < 0.01, log₂ratio > 2; diamond: **p < 0.01, log₂ratio <-2; circle: p >0.01, |log₂ ratio| < 2; square: genes involved in the multi-copy 70k region with ***p < 0.001. (C) Expression Profile of SM1 sorted by metabolic pathways. TPM (Transcripts per million) was deduced by RNA-seq of SM1 during log phase growth (OD600 =0.7). (D) Growth phenotype of SM1 strain re-expressing tpi, gltA, proQ, ptsH pfkA, frmA, ptsP, pgi, gapA in 400 mM methanol. n=3. (E) Specific Activity of Pgi and Pgi mutant (V236_H249del). (F) Growth of SM1 strain in various methanol concentrations. (G) Fermentation profile of SM1. Lines represent growth (circle), methanol consumption (diamond), formate (triangle) and acetate (square). All error bars are defined as standard deviations, n=3. See also FIG. 13.

FIGS. 8A-E shows Construct and evolve a methanol auxotroph strain, related to FIG. 1. (A) Methanol auxotrophy scheme. (B) Two synthetic operons integrated in CFC381.0. “SS3” refers to a safe spot for genome integration. (C) Bioprospecting Hps. Other than Bacillus methanolicus Hps, bioprospecting was performed another Hps was identified from Methylomicrobium buryatense 5GB1S. Specific activity was tested with a coupled assay with rpiA, feeding a fixed amount (2 mM) of either formaldehyde or R5P. Noticeably, the Hps (Mb) has higher activity under low concentrations of R5P, though performs worse in reacting with formaldehyde. The bars represent biologically independent triplicate mean value with error bars as the standard deviation. (D) Growth curve showing the evolution of CFC381 in in HDA media with 400 mM methanol and 20 mM xylose (HMX). (E) Growth curve showing the evolution of CFC381 in MOPS with 400 mM methanol and 20 mM xylose (MMX), after evolution in HMX for 10 generations.

FIGS. 9A-B shows Evolve a synthetic methylotrophic strain, related to FIG. 3. (A) Detailed flowchart of the entire evolution process to enable E. coli to grow on methanol as the sole carbon source. Note that aside from the methylotrophic strain SM1, a non-methylotrophic strain BB1 was also isolated in the final mixed culture that can grow on methanol. (B) Growth curve that shows the evolution of CFC526.23-53 in 400 mM methanol with nitrate.

FIGS. 10A-C shows Further Characterization of DPC in methanol growing strains, related to FIG. 4. (A) SDS-PAGE analysis of proteins extracted from DPC. There is a clear trend that DPC accumulates when OD600 increases. Although the pattern of the bands looks similar, the amount of DPCs detected varies among samples. (B) Growth curve of CFC526.41 and its offspring CFC526.42 growing in 200 mM methanol. No lag phase observed after inoculation of 562.42. (C) TEM images of DNA/DPCs extracted from E. coli cultures grown in different conditions. The LB 526 and LB BW25113 samples are controls for the experiment. Note that lower methanol concentrations (200 mM) alleviated DPC.

FIGS. 11A-B shows Detailed Proteomics data of proteins extracted from DPCs, related to FIG. 4. (A) Complete heat map of the common top 61 hits. The map is ranked by average protein abundance at stationary phase. Note that the deoxyribonuclease (DNAS) entry is an externally added enzyme used for DNA clean up and an internal standard. (B) Individual top 100 hits. The DNAS data is omitted.

FIG. 12 shows Strain characterization of methylotrophic E. coli, related to FIG. 5. Relationships between evolution cultures that are sequenced by Illumina Miseq/ Hiseq. Only mutations that contribute to SM1 were annotated.

FIGS. 13A-B shows Growth phenotype of methylotrophic E. coli, related to FIG. 7. (A) SM1 metabolic flexibility in switching LB & methanol media. The “L” (Grey dot) and “M” (white dot) represent LB medium and methanol MOPS media data respectively. Strains are passed at an inoculation volume of 100 ul with initial OD600 of 0.05. (B) SM1 growing in 400 mM methanol without nitrate or vitamin. SM1 can be stably passed in a minimal media with methanol as the sole carbon source, without any supply of nitrate or vitamin. Strains are passed when it reached OD₆₀₀=1 with an initial OD₆₀₀=1.

FIGS. 14A-B shows Long-read sequencing methylotrophic E. coli, related to FIG. 5. (A) Pacbio and Nanopore sequencing established the genomic structure of the 70k repeated region. The longest read from Pacbio Sequel that mapped between the 70k tandem repeat is 34k, while the longest read from Nanopore that mapped between tandem repeats is 110 kb-long. The latter proves the presence of an at-minimum triplicated 70k tandem-repeat. (B) Mummer plot comparing SM1 and BW25113. The SM1 genome is acquired by de novo assembly from Pacbio sequel data. The main contig highly correlates with the WT genome, suggesting that data is reliable. Moreover, there are two more contigs, including the plamid and interesting, the 70k region. Note that the 70k aligns well on the BW25113 with a breaking point, due to the lack of the synthetic promoter that is integrated in SM1. The plasmid mapped to the WT rpiA position as excepted.

FIGS. 15A-C shows (A) ethanol, (B) succinate and (C) lactate production of methylotrophic E. coli. A titer of more than 2 mM was achieved, detected by Gas Chromatography -Flame Ionization Detector and Liquid Chromatography - Tandem Mass Spectroscopy.

FIGS. 16A-B provide tables showing the natural fermentation products that can be produced by SM1. All products were detected by Liquid Chromatography-Orbitrap Mass Spectroscopy and confirmed with MS/MS metabolomics database. (A) shows products detected in positive mode, while (B) shows products in negative mode.

### DETAILED DESCRIPTION

As used herein and in the appended claims, the singular forms “a,” “an,” and “the” include plural referents unless the context clearly dictates otherwise. Thus, for example, reference to “a polynucleotide” includes a plurality of such polynucleotides and reference to “the microorganism” includes reference to one or more microorganisms, and so forth.

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

Any publications discussed above and throughout the text are provided solely for their disclosure prior to the filing date of the present application. Nothing herein is to be construed as an admission that the inventors are not entitled to antedate such disclosure by virtue of prior disclosure.

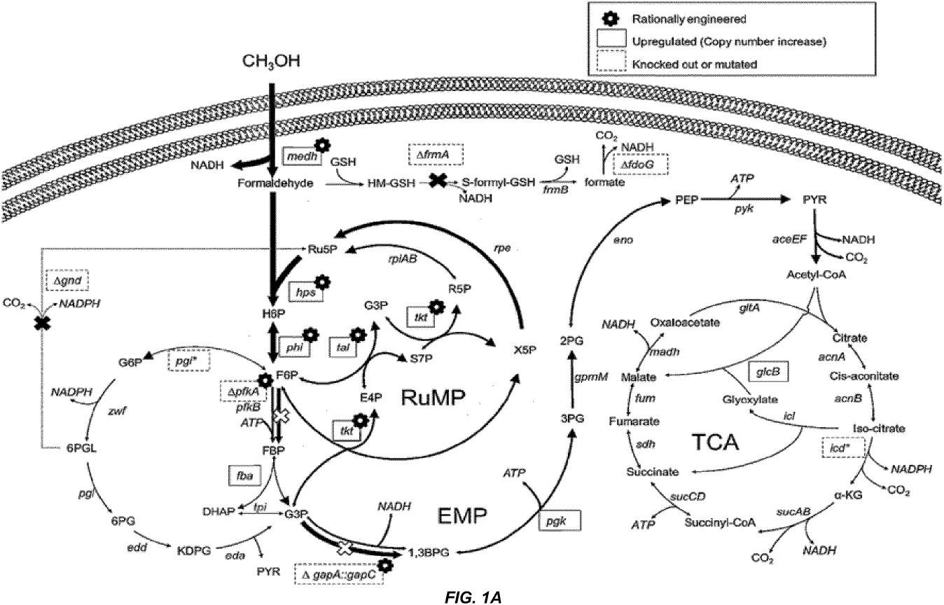

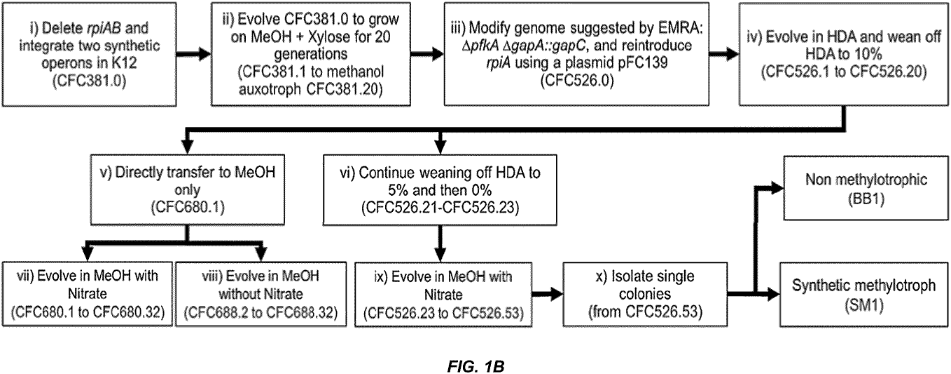

One-carbon (C1) compound assimilation by microorganisms has emerged as a promising approach in abating climate change. Among all C1 compounds, methanol is the most electron-rich in the liquid form, which avoids the diffusion barrier compared to gaseous C1 compounds, methane or CO₂. In addition, methanol is currently an industrial feedstock chemical, ready to use in bioconversion with minimal infrastructural changes. The native methanol utilization and conversion pathways in natural methylotrophs such as Methylobacterium extorquens and Bacillus methanolicus have been well-characterized. These organisms typically utilize the RuMP cycle or the serine pathway for methanol assimilation. In particular, the enzymes involved in the RuMP cycle overlap with those used in the typical sugar metabolism (FIG. 1A and Table 1), except three enzymes (methanol dehydrogenase, Medh; hexulose-6-phosphate synthase, Hps; 6-phospho-3-hexuloisomerase, Phi). Thus, significant efforts have been made to convert sugar heterotrophs to methylotrophs for both scientific and industrial interests by overexpressing these three enzymes.

TABLE 1

Metabolites and genes list, related to FIG. 1 :

| | | | |
|---|---|---|---|
| Metabolite Acronym | Full metabolite name | Gene | Encoding enzyme |
| G6P | Glucose 6-phosphate | zwf | NADP⁺-dependent glucose-6-phosphate dehydrogenase |
| 6PGL | 6-phospho D-glucono-1,5-lactone | gnd | 6-phosphogluconate dehydrogenase |
| 6PG | D-gluconate 6-phosphate | pgl | 6-phosphogluconolactonase |
| KDPG | 2-dehydro-3-deoxy-D-gluconate 6-phosphate | edd | Phosphogluconate dehydratase |
| PYR | pyruvate | eda | 2-keto-3-deoxygluconate 6-phosphate aldolase |
| DHAP | Dihydroxyacetone phosphate | tpi | Triose-phosphate isomerase |
| HM-GSH | hydroxymethyl glutathione | medh | methanol dehydrogenase |
| S-formyl-GSH | S-formylglutathione | frmA | S-(hydroxymethyl)glutathione dehydrogenase |
| H6P | Hexulose-6-phosphate synthase | frmB | S-formylglutathione hydrolase |
| F6P | fructose 6-phosphate | phi | 6-phospho-3-hexuloisomerase |
| FBP | Fructose-1,6-bisphosphate | fdoG | formate dehydrogenase-O |
| G3P | 3-phospho-D-glycerate | pfkA | ATP-dependent 6-phosphofructokinase |
| 1,3BPG | 1,3-Bisphosphoglycerate | pfkB | ATP-dependent 6-phosphofructokinase isozyme |
| 3PG | 3-phosphoglycerate | gapA | glyceraldehyde-3-phosphate dehydrogenase A |
| 2PG | 2-phosphoglycerate | gapC | glyceraldehyde-3-phosphate dehydrogenase |
| PEP | Phosphoenolpyruvate | pgk | phosphoglycerate kinase |
| G3P | Glycerol-3-phosphate | rpiAB | ribose-5-phosphate isomerase |
| E4P | Erythrose 4-phosphate | gpmM | cofactor-independent phosphoglycerate mutase |
| S7P | Sedoheptulose-7-phosphate | eno | enolase |
| R5P | Ribose-5-phosphate | pyk | pyruvate kinase |
| Ru5P | Ribulose 5-phosphate | aceE F | pyruvate dehydrogenase |
| α-KG | Alpha-Ketoglutarate | tal | transaldolase |
| | | tkt | transketolase |
| | | rpe | ribulose-phosphate 3-epimerase |
| | | glta | citrate synthase |
| | | acnA | aconitate hydratase A |
| | | acnB | aconitate hydratase B |
| | | icd | isocitrate dehydrogenase |
| | | sucA B | 2-oxoglutarate dehydrogenase |
| | | sucC D | succinyl-CoA synthase |
| | | frd | succinic dehydrogenase |
| | | fum | fumarase |
| | | madh | malate dehydrogenase |
| | | glcB | malate synthase |
| | | icl | isocitrate lyase |

Despite initial successes in engineering sugar heterotrophs to assimilate methanol, it has not been possible to convert such heterotrophs to methylotrophs that utilize methanol as the sole carbon and energy source efficiently. Reported examples either required other carbon sources or nutrients in the medium to support growth, or demonstrated minimal growth with a doubling time of 55 hours and a maximum OD₆₀₀ of 0.2 with methanol alone. Apparently, successful expression of three heterologous genes is insufficient to turn non-methyltrophs such as, for example, E. coli, into a methylotroph.

This disclosure identifies a major problem involving DNA-protein crosslinking (DPC) that prevented E. coli from growing in methanol as the sole carbon source, and how genome editing, copy number variations, and mutations from evolution overcame this hurdle, resulting in a synthetic methylotrophic E. coli that grows to a high Optical Density (OD) efficiently with a doubling time of 12 hrs or less (e.g., 11.8, 11.6, 11.4, 11.2, 11.0, 10.8, 10.6, 10.4, 10.2, 10, 9.8, 9.6, 9.4, 9.2, 9.0, 8.8, 8.6. 8.4, 8.2, 8.0, 7.8, 7.6, 7.4, 7.2, 7.0, 6.8, 6.6, 6.4, 6.2, 6.0, 5.8, 5.6, 5.4, 5.2, 5.0, 4.8, 4.6, 4.4, 4.2, 4.0, 3.8, 3.6, 3.4, 3.2, 3.0, 2.8, 2.6, 2.4, 2.2, 2.0 hrs. etc. and any value between any of the two foregoing values).

This disclosure demonstrates the tropism change of a microorganism. With only three missing genes of the RuMP cycle (methanol dehydrogenase, Medh; hexulose-6-phosphate synthase, Hps; 6-phospho-3-hexuloisomerase, Phi), the metabolic rewiring turns out to be unexpectedly intricate to convert a microorganism to a methylotroph. Experiments began from a methanol auxotrophy strategy that established the working pathway for methanol assimilation, but the regeneration of the co-substrate, Ru5P, for formaldehyde conversion was supplied from an external carbon source, xylose. This methanol auxotrophy strain was evolved to grow very well with one sixth of its carbon derived from methanol. The remaining task was to wean off xylose and regenerate Ru5P by diverting part of the glycolytic flux to the RuMP cycle. Unexpectedly, this task was challenging, and yet most revealing one in converting a non-methylotroph, e.g., E. coli, to a synthetic methylotroph. In the early stage of evolution for methanol auxotrophic growth (CFC381.20), the formaldehyde detoxification gene, frmA, was inactivated by a frameshift mutation to direct the formaldehyde flux to the productive RuMP pathways.

The disclosure demonstrates that methylotrophic growth on methanol requires a proper balance between RuMP cycle, glycolysis, pentose phosphate pathway, and the ED pathway, imbalance among these pathways causes the shortage of either Ru5P for formaldehyde assimilation, pyruvate for building blocks, or NADPH for biosynthesis. Shortage of Ru5P will result in formaldehyde-induced DPC and then cell death. Shortage of pyruvate or NADPH will hamper growth. Analysis using Ensemble Modeling for Robustness Analysis (EMRA) (Lee et al., 2014; Rivera et al. 2015) was performed and the results suggested that Pfk and Gapdh need to be down regulated in order to avoid severe imbalance among different pathways. Pfk catalyzes a major metabolic step involved in ATP consumption and tunes glycolysis and gluconeogenesis, while Gapdh is a key metabolic node involved in NADH generation and is a junction among glycolysis, RuMP cycle and the pentose phosphate pathway. After implementing EMRA-suggested genomic changes, the cells were able to gain growth advantage in methanol and evolve towards methylotrophic growth. Without these genomic edits demonstrated by the disclosure, the cells appeared to be trapped by DPC and not be able to evolve at the time scale of interest.

The DPC problem was visualized by transmission electron microscopy (TEM), clearly demonstrating the difficulty in turning cells, such as E. coli, to growth in methanol. The DPC phenomenon was most significant in the stationary phase. Even when the cells were able to grow in methanol, DPC kills cells in the stationary phase. Since DPC occurred in a large number of proteins, mutations in protein sequences are not a feasible solution. Typical microbes detoxify formaldehyde by oxidizing it to CO₂, but this strategy wastes the biosynthetic carbon source. For methylotrophy, the organism needs to achieve a fine balance among formaldehyde generation and formaldehyde consumption flux. Native methylotrophs presumably have achieved this fine regulation through natural evolution.

Throughout evolution, divergence created sub-populations identified by genome sequencing. Reviewing this divergence two main populations were identified, the methylotrophic SM1 and the non-methylotrophic BB1 strain (Table 2). SM1 grows on methanol and produces acetate in the late growth phase, which may feed the BB1 strain for growth.

TABLE 2

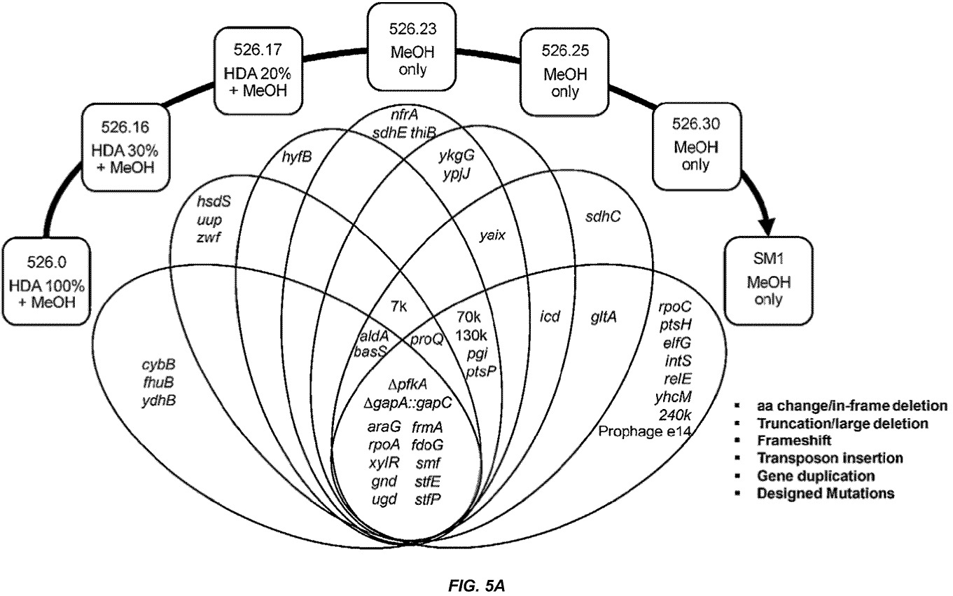

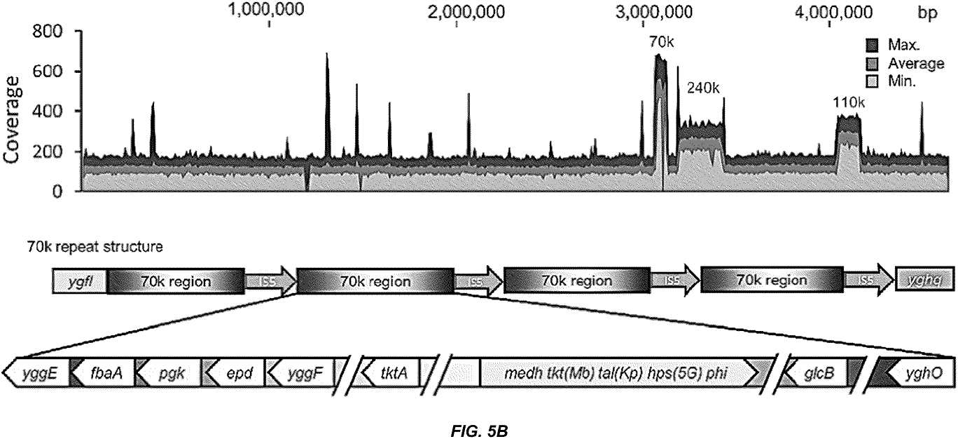

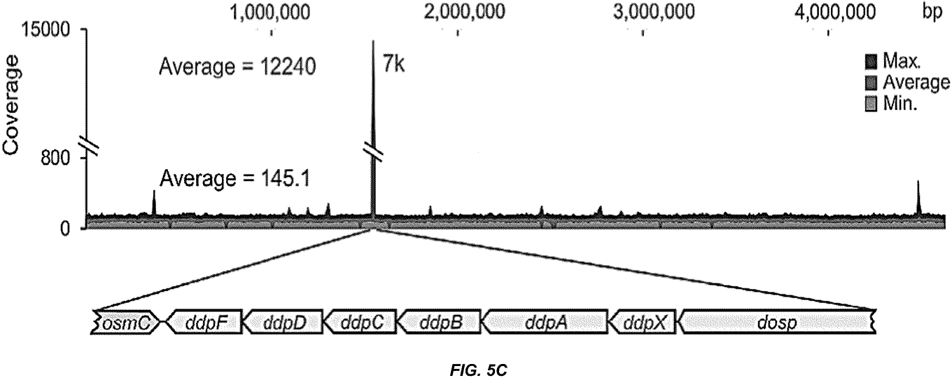

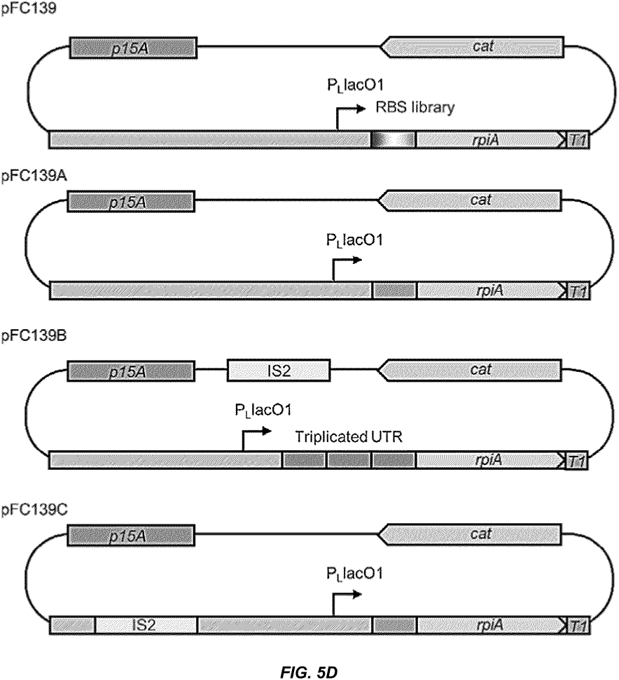

Genotype of strains and cultures (See, FIGS. 1 and 5 )

| | | | | | |
|---|---|---|---|---|---|
| | Codon Change | Noncoding Region | Indel Codon Change | Large Genome Truncation | Copy number Variation (CNV) |
| CFC381.0 | n/a | n/a | n/a | ΔrpiA ΔrpiB | n/a |
| CFC381.2 0 (Same as CFC381.0 except the list to the right) | araG (I275R) rpoA (G315C) xyIR (K320Q) | n/a | fdoG (2,194_del G) [Frame shift] | 2,093,044-2,095,229 ugd N-terminal and operon and, wbbL deletion | 70k (ygge to yghO) duplicate |
| | Low frequency SNVs: cybB (Y69S) fhu (M484G) ydhB (A45G) ydhB (V46G) ydhB (P47A) | | frmA (383_ins CCCG) [Frame shift] | | |
| | | | ugd (1-51_del) [Early stop codon] | | |
| | | | smf (198_ins IS2) [Early stop codon] | stfP_291-stfE_3 transversion | |
| CFC526.0 (Same as CFC381.2 0 except the list to the right) | n/a | n/a | n/a | ΔgapA::gapC ΔptkA | n/a |
| SM1 (Same as CFC526.0 except the list to the right) | rpoC (S733F) proQ (E12*) icd (D398E) icd (D410E) | gItA upstream (IS2 insertion at 750,112) [Disrupts promoter] | pgi (705_del GTTGCAAAA CAC) [Codon:236-239_deIVAKH ] | 1,191,676-1,206,868 cryptic prophage e14 deletion | 70k (ygge to yghO) 4 fold |
| | absence of the low frequency SNVs | ptsH upstream (IS2 insertion at 2,527,069 ) [Disrupts promoter] | relE (202_ins IS2) [Early stop codon] | | 130k (rrsA to rrlB) duplicate |
| BB1 (Same as CFC526.0 except the list to the right) | dacC (V298M) glpR (T22I) rpoA (A267T) | yihL upstream (IS4 insertion at 4053628) [Disrupts promoter] | alsC (59_ins A) [Early Stop Codon] | | 70k (ygge to yghO) 1 fold |
| | | | frmA (384_del T) [Back to in-frame] | | |
| | | | ompF (106_del T) [Frame shift] | | 7k (osmC to dosP) 85 fold |

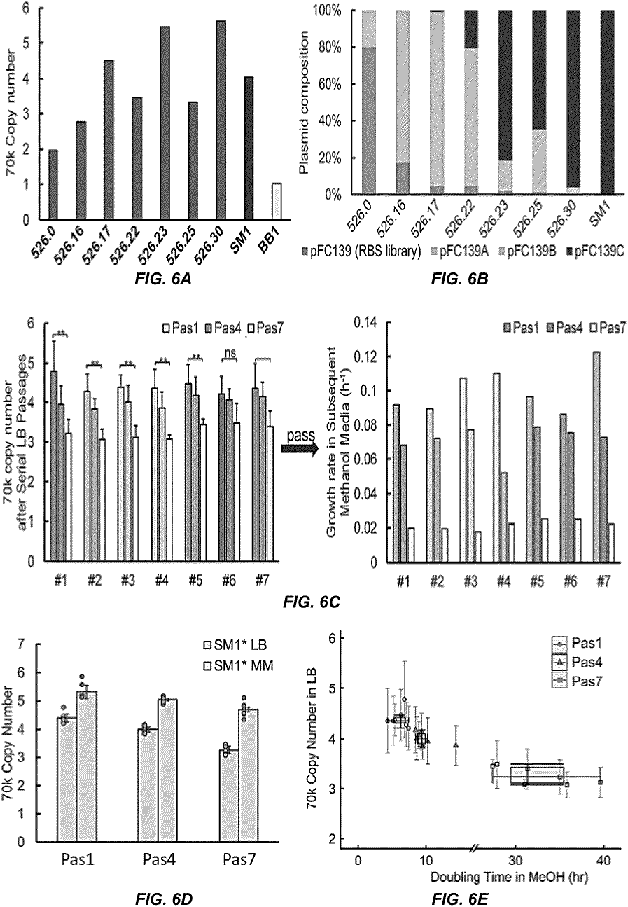

An intriguing feature that laboratory evolution used to solve the DPC problem is copy number variation (CNV). In the SM1 strain, the copy number of the 70K repeated region increased as the evolution proceeded. The isolated SM1 strain showed that the copy of the 70K region decreased when the strain was cultured in LB, but increased when changing from LB to methanol minimal medium (FIG. 6D). This phenomenon was observed in all colonies tested, which disfavored the mixed-population hypothesis. It appears that SM1 uses CNV dynamically to adapt to new environment. The co-evolved non-methylotrophic BB1 strain does not contain the high copy 70k region, but acquired an extremely high (85) copy 7k region flanked by IS. It implies that IS-mediated CNV plays an important role in laboratory evolution for adapting to a challenging environment. E. coli dynamically tuned CNV along with environmental changes.

Moreover, the copy number for the 70k region in the initial CFC526.0 is already 2, indicating that this CNV may have occurred since methanol auxotrophy evolution. Thus, this may also explain why the stepwise evolution strategy is effective. Without this auxotroph strategy to prepare the genomic background, the 70k region may not have become available for further copy number increase and optimization.

After evolution, the final synthetic methylotroph strain exhibits doubling time (t_{D}) of about 8.5 hours and methanol tolerance (up to 1.2 M) comparable to native methylotrophs such as Methylobacterium extorquens AM1, (t_{D} = 4 hr (Nayak and Marx, 2014)), Methylobacterium extorquens TK0001 (t_{D} = 4~6 hr (Belkhelfa et al., 2019)) and Pichia pastoris (t_{D} = 8.2 hr (Moser et al., 2017)) in methanol only conditions.

The disclosure provides for reprogrammed prokaryotic microorganisms, such as E. coli, using metabolic robustness criteria followed by laboratory evolution to establish a strain(s) that can utilize methanol as the sole carbon source efficiently. This “synthetic methylotroph” overcomes a heretofore uncharacterized hurdle, DNA-protein crosslinking (DPC), by insertion sequence (IS) mediated copy number variations (CNV) and balancing the metabolic flux by mutations. The synthetic methylotrophs are capable of growing at a rate comparable to natural methylotrophs in a wide-range of methanol concentrations, these synthetic methylotrophic strain(s) illustrate genome editing and evolution for microbial tropism changes, and expands the scope of biological C1 conversion. The disclosure provides a solution to the problems identified above by introducing two genome edits followed by laboratory evolution.

The disclosure provides a synthetic methylotroph strain having a doubling time (t_{D}) of less than 12 hours (e.g., less than 11 hours, 10 hours, 9 hours, 8 hours, 7 hours, 6 hours, 5 hours, 4 hours, 3 hours, 2 hours etc. and any value between any of the foregoing two values) comparable to native methylotrophs such as Methylobacterium extorquens AM1, (t_{D} = 4 hr), Methylobacterium extorquens TK0001 (t_{D} = 4~6 hr) and Pichia pastoris (t_{D} = 8.2 hr) in methanol only conditions. In one embodiment, the disclosure provides a synthetic methylotroph comprising enzymes of the RuMP cycle and further including a methanol dehydrogenase, a hexulose-6-phosphate synthase and a hexulose-6-phosphate isomerase.

The terms “methylotroph,” “methylotrophic microorganism” and “methylotrophic microbe” are used herein interchangeably and refer to a microbe capable of metabolizing a one-carbon compound (e.g., an organic-carbon compound), such as methane or methanol, into its cell mass, a metabolite or a combination thereof.

The terms “non-methylotroph,” “non-methylotrophic microorganism” and “non-methylotrophic microbe” are used herein interchangeably and refer to a microbe incapable of metabolizing a one-carbon compound, such as methane or methanol, into its cell mass, a metabolite or a combination thereof.

The terms “non-naturally occurring methylotroph,” “non-naturally occurring methylotrophic microorganism” and “synthetic methylotroph” are used herein interchangeably and refer to a methylotroph that has been prepared by modifying one or more native genes and/or expressing one or more heterologous genes in a non-methylotroph and/or synthetically evolving the microorganism such that it comprises genotypic differences compared to a parental microorganism. Stated differently, a “synthetic methylotroph” refers to a microorganism derived from a parental microorganism that lacks the ability grow efficiently or to grow at all on an organic C1 carbon source, but through recombinant engineering or recombinant engineering and laboratory evolution is engineered and adapted to grow on an organic C1 carbon sources such as methanol.

A synthetic methylotroph is a recombinant microorganism selected from the group consisting of facultative aerobic organisms, facultative anaerobic organisms and anaerobic organisms that have been engineered to utilize an organic C1 carbon source into its cell mass. The synthetic methylotroph can be engineered from a parental microbe selected from the group consisting of phyla Proteobacteria, Firmicutes, Actinobacteria, Cyanobacteria, Chlorobi and Deinococcus-Thermus. In some embodiments, the synthetic methylotroph is a microbe engineered from a parental microbe selected from the group consisting of Acetobacter, Acinetobacter, Bacillus, Chlorobi, Clostridium, Corynebacterium, Cyanobacteria, Deinococcus, Enterobacter, Enterobacteria, Escherichia, Geobacillus, Geobacter, Klebsiella, Lactobacillus, Lactococcus, Mannheimia, Propionibacterium, Pseudomonas, Ralstonia, Shewanella, Streptococcus, Streptomyces, Synechococcus, Synechocystis and Zymomonas. In one embodiment, the synthetic methylotroph is engineered from a parental Escherichia coli.

In one embodiment, a synthetic methylotroph provided herein includes elevated expression of a hexulose-6-phosphate synthase as compared to a parental microorganism. This expression may be combined with the expression or over-expression with other enzymes in the metabolic pathway to metabolize/assimilate, and grow on an organic C1 carbon source. The recombinant microorganism produces a metabolite that includes hexulose-6-phosphate from formaldehyde and ribulose-5-phosphate. The hexulose-6-phosphate synthase can be encoded by an hps gene, polynucleotide or homolog thereof. The hps gene or polynucleotide can be derived from various microorganisms including B. subtilis.

In addition to the foregoing, the terms “hexulose-6-phosphate synthase” or “Hps” refer to proteins that are capable of catalyzing the formation of hexulose-6-phosphate from formaldehyde and ribulose-5-phosphate, and which share at least about 40%, 45%, 50%, 55%, 60%, 65%, 70%, 75%, 80%, 85%, 90%, 95%, 96%, 97%, 98%, 99% (or a value between any two of the foregoing values) or greater sequence identity, or at least about 50%, 60%, 70%, 80%, 90%, 95%, 96%, 97%, 98%, 99% (or a value between any two of the foregoing values) or greater sequence similarity, as calculated by NCBI BLAST, using default parameters, to SEQ ID NO:2.

In another or further embodiment, a synthetic methylotroph provided herein includes elevated expression of a hexulose-6-phosphate isomerase as compared to a parental microorganism. This expression may be combined with the expression or over-expression with other enzymes in the metabolic pathway to metabolize/assimilate, and grow on an organic C1 carbon source. The recombinant microorganism produces a metabolite that includes fructose-6-phosphate from hexulose-6-phosphate. The hexulose-6-phosphate isomerase can be encoded by a phi gene, polynucleotide or homolog thereof. The phi gene or polynucleotide can be derived from various microorganisms including M. Flagettus.

In addition to the foregoing, the terms “hexulose-6-phosphate isomerase” or “Phi” refer to proteins that are capable of catalyzing the formation of fructose-6-phosphate from hexulose-6-phosphate, and which share at least about 40%, 45%, 50%, 55%, 60%, 65%, 70%, 75%, 80%, 85%, 90%, 95%, 96%, 97%, 98%, 99% (or a value between any two of the foregoing values) or greater sequence identity, or at least about 50%, 60%, 70%, 80%, 90%, 95%, 96%, 97%, 98%, 99% (or a value between any two of the foregoing values) or greater sequence similarity, as calculated by NCBI BLAST, using default parameters, to SEQ ID NO:4.

In another or further embodiment, a recombinant microorganism provided herein includes elevated expression of methanol dehydrogenase (Mdh, also referred to as Medh) as compared to a parental microorganism. This expression may be combined with the expression or over-expression with other enzymes in a pathway to metabolize/assimilate, and grow on an organic C1 carbon source. The recombinant microorganism produces a metabolite that includes formaldehyde from a substrate that includes methanol. The methanol dehydrogenase can be encoded by an medh gene, polynucleotide or homolog thereof. The medh gene or Medh polynucleotide can be derived from various microorganisms including B.methanolicus.

In addition to the foregoing, the terms “methanol dehydrogenase” or “Mdh” or “Medh” refer to proteins that are capable of catalyzing the formation of formaldehyde from methanol, and which share at least about 40%, 45%, 50%, 55%, 60%, 65%, 70%, 75%, 80%, 85%, 90%, 95%, 96%, 97%, 98%, 99% (or a value between any two of the foregoing values) or greater sequence identity, or at least about 50%, 60%, 70%, 80%, 90%, 95%, 96%, 97%, 98%, 99% (or a value between any two of the foregoing values) or greater sequence similarity, as calculated by NCBI BLAST, using default parameters, to SEQ ID NO:6.

In another embodiment, a recombinant microorganism provided herein includes elevated expression of transaldolase as compared to a parental microorganism. This expression may be combined with the expression or over-expression with other enzymes in the metabolic pathway to metabolize/assimilate, and grow on an organic C1 carbon source. The recombinant microorganism produces a metabolite that includes sedoheptulose-7-phosphate from a substrate that includes erythrose-4-phosphate and fructose-6-phosphate. The transaldolase can be encoded by a tal gene, polyncleotide or homolog thereof. The tal gene or polynucleotide can be derived from various microorganisms including E. coli.

In addition to the foregoing, the terms “transaldolase” or “Tal” refer to proteins that are capable of catalyzing the formation of sedoheptulose-7-phosphate from erythrose-4-phosphate and fructose-6-phosphate, and which share at least about 40%, 45%, 50%, 55%, 60%, 65%, 70%, 75%, 80%, 85%, 90%, 95%, 96%, 97%, 98%, 99% (or a value between any two of the foregoing values) or greater sequence identity, or at least about 50%, 60%, 70%, 80%, 90%, 95%, 96%, 97%, 98%, 99% (or a value between any two of the foregoing values) or greater sequence similarity, as calculated by NCBI BLAST, using default parameters, to SEQ ID NO:17. Additional homologs include: Bifidobacterium breve DSM 20213 ZP_06596167.1 having 30% identity to SEQ ID NO:17; Homo sapiens AAC51151.1 having 67% identity to SEQ ID NO:17; Cyanothece sp. CCY0110 ZP_01731137.1 having 57% identity to SEQ ID NO:17; Ralstonia eutropha JMP134 YP_296277.2 having 57% identity to SEQ ID NO:17; and Bacillus subtilis BEST7613 NP_440132.1 having 59% identity to SEQ ID NO:17. The sequences associated with the foregoing accession numbers are incorporated herein by reference.

In another embodiment, a recombinant microorganism provided herein includes elevated expression of transketolase as compared to a parental microorganism. This expression may be combined with the expression or over-expression with other enzymes in a pathway to metabolize/assimilate, and grow on an organic C1 carbon source such as methanol. The recombinant microorganism produces a metabolite that includes (i) ribose-5-phosphate and xylulose-5-phosphate from sedoheptulose-7-phosphate and glyceraldhyde-3-phosphate; and/or (ii) glyceraldehyde-3-phosphate and fructose-6-phosphate from xylulose-5-phosphate and erythrose-4-phosphate. The transketolase can be encoded by a tkt gene, polyncleotide or homolog thereof. The tkt gene or polynucleotide can be derived from various microorganisms including E. coli.

In addition to the foregoing, the terms “transketolase” or “Tkt” refer to proteins that are capable of catalyzing the formation of (i) ribose-5-phosphate and xylulose-5-phosphate from sedoheptulose-7-phosphate and glyceraldhyde-3-phosphate; and/or (ii) glyceraldehyde-3-phosphate and fructose-6-phosphate from xylulose-5-phosphate and erythrose-4-phosphate, and which share at least about 40%, 45%, 50%, 55%, 60%, 65%, 70%, 75%, 80%, 85%, 90%, 95%, 96%, 97%, 98%, 99% (or a value between any two of the foregoing values) or greater sequence identity, or at least about 50%, 60%, 70%, 80%, 90%, 95%, 96%, 97%, 98%, 99% (or a value between any two of the foregoing values) or greater sequence similarity, as calculated by NCBI BLAST, using default parameters, to SEQ ID NO:19. Additional homologs include: Neisseria meningitidis M13399 ZP_11612112.1 having 65% identity to SEQ ID NO: 19; Bifidobacterium breve DSM 20213 ZP_06596168.1 having 41% identity to SEQ ID NO:19; Ralstonia eutropha JMP134 YP_297046.1 having 66% identity to SEQ ID NO: 19; Synechococcus elongatus PCC 6301 YP_171693.1 having 56% identity to SEQ ID NO: 19; and Bacillus subtilis BEST7613 NP_440630.1 having 54% identity to SEQ ID NO: 19. The sequences associated with the foregoing accession numbers are incorporated herein by reference.

In another embodiment, a recombinant microorganism provided herein includes elevated expression of a fructose 1,6 bisphosphate aldolase as compared to a parental microorganism. This expression may be combined with the expression or over-expression with other enzymes in a pathway to metabolize/assimilate, and grow on an organic C1 carbon source such as methanol. The recombinant microorganism produces a metabolite that includes fructose 1,6-bisphosphate from a substrate that includes dihydroxyacetone phosphate and glyceraldehyde-3-phosphate. The fructose 1,6 bisphosphate aldolase can be encoded by a fba gene, polyncleotide or homolog thereof. The fba gene or polynucleotide can be derived from various microorganisms including E. coli.

In addition to the foregoing, the terms “fructose 1,6 bisphosphate aldolase” or “Fba” refer to proteins that are capable of catalyzing the formation of fructose 1,6-bisphosphate from a substrate that includes dihydroxyacetone phosphate and glyceraldehyde-3-phosphate, and which share at least about 40%, 45%, 50%, 55%, 60%, 65%, 70%, 75%, 80%, 85%, 90%, 95%, 96%, 97%, 98%, 99% (or a value between any two of the foregoing values) or greater sequence identity, or at least about 50%, 60%, 70%, 80%, 90%, 95%, 96%, 97%, 98%, 99% (or a value between any two of the foregoing values) or greater sequence similarity, as calculated by NCBI BLAST, using default parameters, to SEQ ID NO:21. Additional homologs include: Synechococcus elongatus PCC 6301 YP_170823.1 having 26% identity to SEQ ID NO: 20; Vibrio nigripulchritudo ATCC 27043 ZP_08732298.1 having 80% identity to SEQ ID NO: 20; Methylomicrobium album BG8 ZP_09865128.1 having 76% identity to SEQ ID NO: 20; Pseudomonas fluorescens Pf0-1 YP_350990.1 having 25% identity to SEQ ID NO: 20; and Methylobacterium nodulans ORS 2060 YP_002502325.1 having 24% identity to SEQ ID NO:20. The sequences associated with the foregoing accession numbers are incorporated herein by reference.

In another embodiment, a system or recombinant microorganism provided herein includes a phosphoglycerate kinase. This enzyme may be combined with the expression or over-expression with other enzymes in a pathway to metabolize/assimilate, and grow on an organic C1 carbon source such as methanol. The enzyme produces a metabolite that includes 3-phosphoglycerate from 1,3-bisphosphoglycerate and ADP. The phosphoglycerate kinase can be encoded by by a pgk gene, polyncleotide or homolog thereof. The pgk gene or polynucleotide can be derived from various microorganisms including G. stearothermophilus.

In addition to the foregoing, the terms “phosphoglycerate kinase” or “Pgk” refer to proteins that are capable of catalyzing the formation of 3-phosphoglycerate from 1,3-bisphosphoglycerate and ADP, and which share at least about 40%, 45%, 50%, 55%, 60%, 65%, 70%, 75%, 80%, 85%, 90%, 95%, 96%, 97%, 98%, 99% (or a value between any two of the foregoing values) or greater sequence identity to SEQ ID NO:22, or at least about 50%, 60%, 70%, 80%, 90%, 95%, 96%, 97%, 98%, 99% (or a value between any two of the foregoing values) or greater sequence similarity, as calculated by NCBI BLAST, using default parameters.

Fructose 6-phosphate (F6P) catalyzed by the enzymes, 3-hexulose-6-phosphate synthase (HPS) and 6-phospho-3-hexuloisomerase (PHI) can then be metabolized via the main metabolic cellular pathways: glycolysis (the EMP pathway), the Entner-Doudoroff (ED) pathway, or Pentose Phosphate Pathway (PPP).

In yet another or further embodiment, a synthetic methylotroph of the disclosure can also benefit from other recombinant engineering processes and genes. For example, in one embodiment, the synthetic methylotroph can benefit from over expression or activity of phosphoglucoisomerase (glucosephosphate isomerase) expression or activity. This expression may be combined with the expression or over-expression with other enzymes in a pathway to metabolize/assimilate, and grow on an organic C1 carbon source. The glucosephosphate isomerase can be encoded by a pgi gene, polynucleotide or homolog thereof. The pgi gene or polynucleotide can be derived from various microorganisms including E. coli.

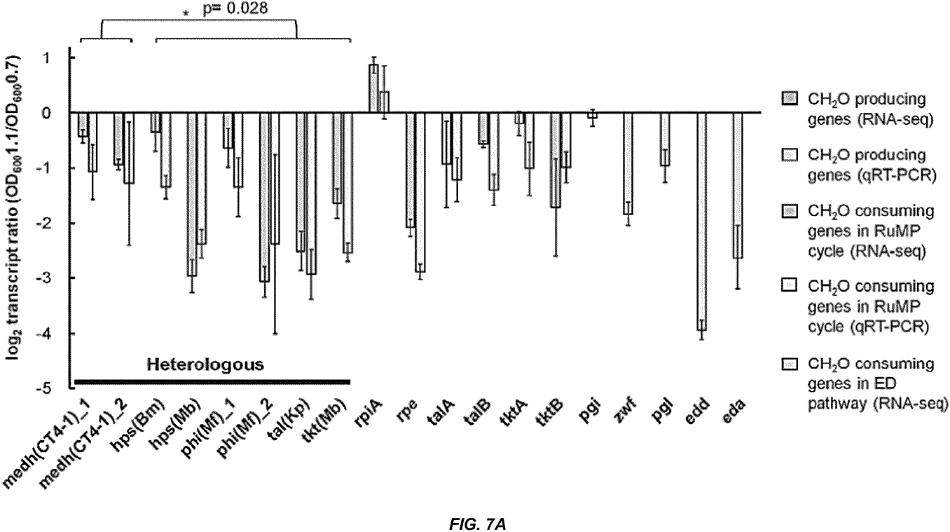

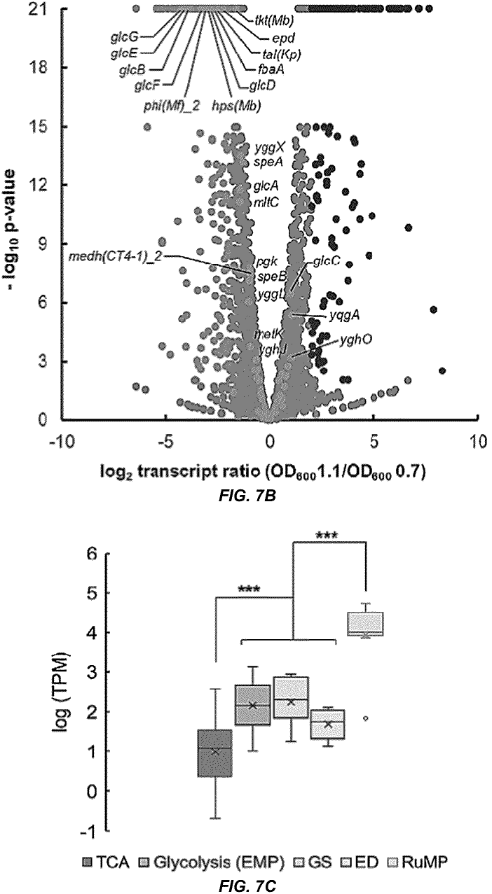

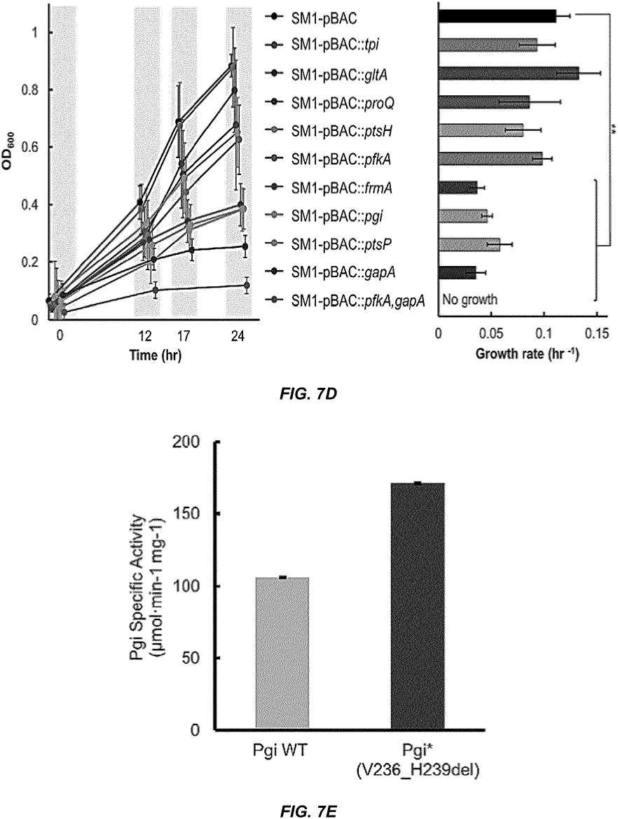

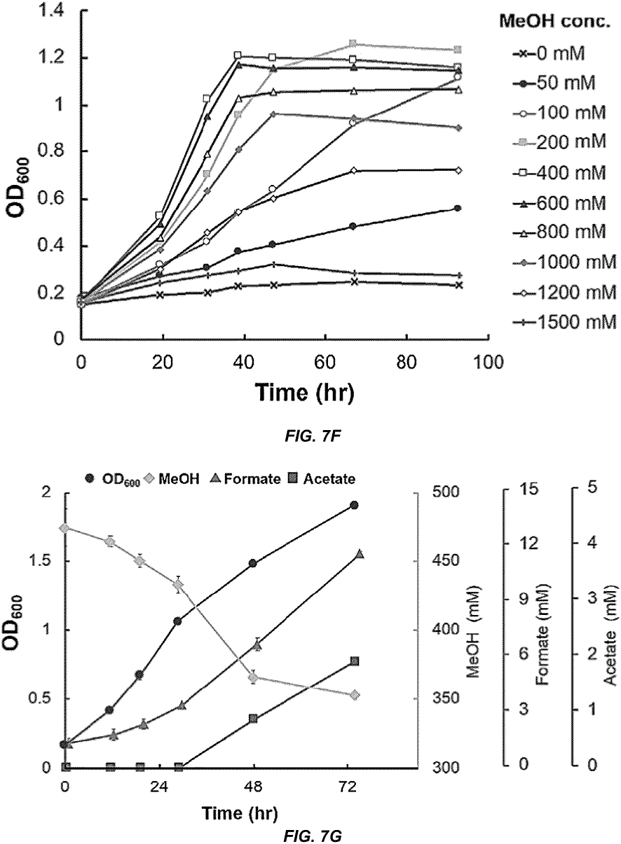

In addition to the foregoing, the terms “phosphoglucose isomerase” or “glucose phosphate isomerase” or “Pgi” refer to proteins that are capable of catalyzing the reversible isomerization of glucose-6phosphate and fructose-6-phosphate, and which share at least about 40%, 45%, 50%, 55%, 60%, 65%, 70%, 75%, 80%, 85%, 90%, 95%, 96%, 97%, 98%, 99% (or a value between any two of the foregoing values) or greater sequence identity, or at least about 50%, 60%, 70%, 80%, 90%, 95%, 96%, 97%, 98%, 99% (or a value between any two of the foregoing values) or greater sequence similarity, as calculated by NCBI BLAST, using default parameters, to SEQ ID NO:8. In one embodiment, the Pgi is a mutant Pgi comprising a 12 bp deletion in the coding sequence which gives rise to a Pgi polypeptide of SEQ ID NO:10 and sequences that are at least 95%-100% identical thereto. For example, the disclosure demonstrates that other mutations, such as a 12-bp deletion in Pgi, which increased its activity (FIG. 7D). It is suspected that Pgi activity diverts part of the flux to the oxidative pentose phosphate pathway and generate NADPH for growth. The carbon flux then feeds into the ED pathway to generate pyruvate for growth and G3P for the RuMP pathway (FIG. 1A).

In another embodiment, the recombinant microorganism has an increased activity or expression of a ribose-5-phosphate isomerase or a homologue or variant thereof. In some embodiments, the ribose-5-phosphate isomerase is ribose-5-phosphate isomerase A. In some embodiments, the ribose-5-phosphate isomerase A is alkali-inducible. An example of ribose-5-phosphate isomerase A is rpiA from E. coli. Ribose 5-phosphate isomerases interconvert ribose 5-phosphate and ribulose 5-phosphate. This reaction allows the synthesis of ribose from the pentose phosphate pathway and represents a system for the salvage of carbohydrates. RpiA is highly conserved and present in almost all organisms. In E. coli, the enzyme is constitutively expressed.

In addition to the foregoing, the terms “ribose-5-phosphate isomerase” or “rpiA” refer to proteins that are capable of interconversion of ribose 5-phosphate and ribulose 5-phosphate, and which share at least about 40%, 45%, 50%, 55%, 60%, 65%, 70%, 75%, 80%, 85%, 90%, 95%, 96%, 97%, 98%, 99% (or a value between any two of the foregoing values) or greater sequence identity, or at least about 50%, 60%, 70%, 80%, 90%, 95%, 96%, 97%, 98%, 99% (or a value between any two of the foregoing values) or greater sequence similarity, as calculated by NCBI BLAST, using default parameters, to SEQ ID NO:14.

In one embodiment, the disclosure provides a recombinant microorganism comprising elevated expression of at least one target enzyme as compared to a parental microorganism or encodes an enzyme not found in the parental organism. For example, the recombinant microorganism (e.g., synthetic methylotroph) can be engineered to express or over-express one or more enzymes selected from the group consisting of Medh, Hps, Phi, Tkt, Tal and Pgi. In a further embodiment, the recombinant microorganism can express or overexpress rpiA or has increased RpiA activity. In one embodiment, the recombinant microorganism is engineered to express Medh, Hps, Phi and a mutant Pgi.

In another or further embodiment, the microorganism comprises a reduction, disruption or knockout of at least one gene encoding an enzyme. In one embodiment, the recombinant microorganism comprises a knockout or disruption of a phosphocarrier protein HPr (also referred to as Histidine-containing protein, HPr and/or ptsH). In a further embodiment, the ptsH polypeptide has a sequence that is at least 95%-100% identical to SEQ ID NO:11. Polynucleotide sequences encoding ptsH can be derived/identified from SEQ ID NO:11 by using well known codon tables and the degeneracy of the genetic code. In another or further embodiment, the recombinant microorganism comprises or further comprises a knockout or disruption in a proQ gene. The proQ gene encodes a polypeptide having a sequence that is at least 95%-100% identical to SEQ ID NO:12. The gene/polynucleotide encoding a polypeptide of SEQ ID NO:12 can be derived/identified by using well known codon tables and the degeneracy of the geneitic code.

In yet another or further embodiment, the recombinant microorganism (e.g., synthetic methylotroph) comprises a reduction or knockout in the expression of a formaldehyde dehydrogenase (frma) or the elimination or reduction in activity of a formaldehyde dehydrogenase (frmA). Various frmAs and their homologs are known, e.g., formaldehyde dehydrogenase (frmA) from E. coli has accession number HG738867. Homologs of frmaA are known; such as formaldehyde dehydrogenase from P. putida having Acc. #CP005976; or from K. pneumoniae having Acc. #D16172; or from D. dadantii having Acc. #CP001654 or from P. stutzeri from Acc. #CP003677 (the sequences of the identified accession numbers are incorporated herein by reference).

In yet another or further embodiment of any of the foregoing, the microorganism can comprise a deletion (knockout) of a glyceraldehyde-3-phosphate dehydrogenase (gapA, or a homolog thereof). In another embodiment, the recombinant microorganism comprises a weakened gapA activity. In a further embodiment, the microorganism comprises a gapC activity that is about 40% (e.g., 32%, 33%, 34%, 35%, 36%, 37%, 38%, 39%, 41%, 42%, 43%, 44%, 45%, 46%, 47%, 48%) of the gap A activity in the wild-type activity. The terms “glyceraldehyde-3-phosphate dehydrogenase A” and “GapA” are used interchangeably herein and refer to a protein having an enzymatic activity capable of catalyzing the conversion of glyceraldehyde 3-phosphate + phosphate + NAD⁺ to 3-phospho-D-glyceroyl-phosphate + NADH + H. Typical glyceraldehyde-3-phosphate dehydrogenases are characterized by EC 1.2.1.12. Glyceraldehyde-3-phosphate dehydrogenase is encoded by gapA in E. coli. In another embodiment, the gapA is replaced with gapC. GapC is a glyceraldehyde-3-phosphate dehydrogenase and can have a sequence that is at least 92%, 95%, 98% (or any value between any two of the foregoing values), or 100% sequence identity to SEQ ID NO:15.

In yet another or further embodiment of any of the foregoing, the microorganism can comprise a reduction or deletion (knockout) of a 6-phosphofructokinase 1 (PfkA, or a homolog thereof). In another embodiment, the recombinant microorganism comprises a weakened PfkA activity. In a further embodiment, the microorganism comprises a PfkB activity that is about 5% (e.g., 2%, 3%, 4%, 5%, 6%, 7%, 8%, 9%, 11%, 12%, 13%, 14%, 15%, 16%, 17%, 18%) of overall Pfk activity in E. coli. The terms “6-phosphofructokinase 1” and “PfkA” are used interchangeably herein and refer to a protein having an enzymatic activity capable of catalyzing the conversion of ATP + β-D-fructose 6-phosphate to ADP + β-D-fructose 1,6-bisphosphate + H⁺. Typical phosphofructokinases are characterized by EC 2.7.1.11. 6-phosphofructokinase 1 is encoded by pfkA in E. coli. A pfkA nucleotides sequence can comprise a sequence that is at least 70-100% identical to SEQ ID NO:41 and encodes a polypeptide that catalyzes the conversion of ATP + β-D-fructose 6-phosphate to ADP + β-D-fructose 1,6-bisphosphate + H⁺. In another embodiment, a PfkA comprises a sequence that is at least 70-100% identical to SEQ ID NO:42 and catalyzes the conversion of ATP + β-D-fructose 6-phosphate to ADP + β-D-fructose 1,6-bisphosphate + H⁺.

In another embodiment, a microorganism that comprises a reduction or knockout of the PfkA is compensated by expression of PfkB. PfkB is a 6-phosphofructokinase 2 and can have a sequence that is at least 92%, 95%, 98% or 100% (or any value between any two of the foregoing values), identical to SEQ ID NO:44.

In still another or further embodiment, a recombinant microorganism (e.g., synthetic methylotroph) of the disclosure comprises a region of the genome having a copy number of greater than 2. For example, in one embodiment, the recombinant microorganism has a copy number of greater than 2 (e.g., 3, 4, 5, 6, 7, 8 to 85 fold) of a region selected from the group consisting of: yggE to yghO, rrsA to rriB, and/or ygiG to smf. In one embodment, the recombinant microorganism comprises a copy number variation of 2, 3, 4 or more of the 70k yggE to yghO region. In certain embodiments, the copy number if a fixed value greater than 2 and less than 90 (and includes any value therebetween as if expressly listed here).

As used herein, the term “metabolically engineered” or “metabolic engineering” involves rational pathway design and assembly of biosynthetic genes, genes associated with operons, and control elements of such polynucleotides, for the production of a desired metabolite or metabolism of a particular substrate. “Metabolically engineered” can further include optimization of metabolic flux by regulation and optimization of transcription, translation, protein stability and protein functionality using genetic engineering and appropriate culture condition including the reduction of, disruption, or knocking out of, a competing metabolic pathway that competes with an intermediate leading to a desired pathway. A biosynthetic gene can be heterologous to the host microorganism, either by virtue of being foreign to the host, or being modified by mutagenesis, recombination, and/or association with a heterologous expression control sequence in an endogenous host cell. In one embodiment, where the polynucleotide is xenogenetic to the host organism, the polynucleotide can be codon optimized.

The term “biosynthetic pathway”, also referred to as “metabolic pathway”, refers to a set of anabolic or catabolic biochemical reactions for converting (transmuting) one chemical species into another. Gene products belong to the same “metabolic pathway” if they, in parallel or in series, act on the same substrate, produce the same product, or act on or produce a metabolic intermediate (i.e., metabolite) between the same substrate and metabolite end product.

The term “substrate” or “suitable substrate” refers to any substance or compound that is converted or meant to be converted into another compound by the action of an enzyme. The term includes not only a single compound, but also combinations of compounds, such as solutions, mixtures and other materials which contain at least one substrate, or derivatives thereof. Further, the term “substrate” encompasses not only compounds that provide a carbon source suitable for use as a starting material, such as a C1 carbon source (e.g., methanol), but also intermediate and end product metabolites used in a pathway associated with a metabolically engineered microorganism as described herein.

Recombinant microorganisms provided herein can express a plurality of target enzymes involved in the use of a C1 carbon source as a substrate (e.g., methanol). The plurality of enzymes are selected from the group consisting of Medh, Hps, Phi, Pgi, rpiA, Tkt, Tal and any combination thereof (at least one of which is heterologous to the recombinant microorganism or expressed at a nonnatural level). In a further embodiment, the recombinant microorganism includes a reduction or knockout of a gene selected from the group consisting of pfkA, gapA, frmA, ptsH, proQ and any combination thereof. In still a further embodiment, the recombinant microorganism includes an amplified (e.g., high copy number (2, 3, 4, 5 to 85)) region of the genome. The recombinant microorganism can grow on a C1 carbon sources such as methanol.

Accordingly, metabolically “engineered” or “modified” microorganisms are produced via the introduction of genetic material into a host or parental microorganism of choice thereby modifying or altering the cellular physiology and biochemistry of the microorganism. Through the introduction of genetic material the parental microorganism acquires new properties, e.g., the ability to produce a new, or greater quantities of, an intracellular metabolite or grow and metabolize a substrate that is not natural for the microorganism. The genetic material introduced into the parental microorganism contains gene(s), or parts of genes, coding for one or more of the enzymes involved in a biosynthetic pathway for using a C1 carbon source for integration into the cell’s mass.

An engineered or modified microorganism can also include in the alternative or in addition to the introduction of a genetic material into a host or parental micoorganism, the disruption, deletion or knocking out of a gene or polynucleotide to alter the cellular physiology and biochemistry of the microorganism. Through the reduction, disruption or knocking out of a gene or polynucleotide the microorganism acquires new or improved properties (e.g., the ability to produced a new or greater quantity of an interacellular metabolite, improve the flux of a metabolite down a desired pathway, and/or reduce the production of undesireable byproducts).

The disclosure demonstrates that the expression of one or more heterologous polynucleotide or over-expression of one or more heterologous polynucleotide encoding a polypeptide having methanol dehydrogenase activity, hexulose-6-phosphate synthase activity, 6-phospho-3-hexulose isomerase activity, glucose phosphate isomerase activity and ribose-phosphate isomerase A activity, with a concomitant reduction or elimination of phosphofructokinase activity, reduction or elimination of glyceraldehyde-3-phosphate dehydrogenase activity, reduction or elimination of S-(hydroxymethyl)glutathione dehydrnase (frmA) activity, reduction or deletion of phosphocarrier protein HPr (also referred to as Histidine-containing protein, HPr and/or ptsH) activity, and the reduction or elimination of of proQ provides a microorganism with the ability to grown on methanol.

Microorganisms provided herein are modified to produce metabolites in quantities not available in the parental microorganism. A “metabolite” refers to any substance produced by metabolism or a substance necessary for or taking part in a particular metabolic process. A metabolite can be an organic compound that is a starting material (e.g., methanol), an intermediate (e.g., glucose-6-phosphate) in, or an end product of metabolism. Metabolites can be used to construct more complex molecules, or they can be broken down into simpler ones. Intermediate metabolites may be synthesized from other metabolites, perhaps used to make more complex substances, or broken down into simpler compounds, often with the release of chemical energy.

The disclosure identifies specific genes useful in the methods, compositions and organisms of the disclosure; however, it will be recognized that absolute identity to such genes is not necessary. For example, changes in a particular gene or polynucleotide comprising a sequence encoding a polypeptide or enzyme can be performed and screened for activity. Typically, such changes comprise conservative mutation and silent mutations. Such modified or mutated polynucleotides and polypeptides can be screened for expression of a function enzyme activity using methods known in the art.

Due to the inherent degeneracy of the genetic code, other polynucleotides which encode substantially the same or a functionally equivalent polypeptide can also be used to clone and express the polynucleotides encoding such enzymes.

As will be understood by those of skill in the art, it can be advantageous to modify a coding sequence to enhance its expression in a particular host. The genetic code is redundant with 64 possible codons, but most organisms typically use a subset of these codons. The codons that are utilized most often in a species are called optimal codons, and those not utilized very often are classified as rare or low-usage codons. Codons can be substituted to reflect the preferred codon usage of the host, a process sometimes called “codon optimization” or “controlling for species codon bias.”

Optimized coding sequences containing codons preferred by a particular prokaryotic or eukaryotic host (see also, Murray et al. (1989) Nucl. Acids Res. 17:477-508) can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. For example, typical stop codons for S. cerevisiae and mammals are UAA and UGA, respectively. The typical stop codon for monocotyledonous plants is UGA, whereas insects and E. coli commonly use UAA as the stop codon (Dalphin et al. (1996) Nucl. Acids Res. 24: 216-218). Methodology for optimizing a nucleotide sequence for expression in a plant is provided, for example, in U.S. Pat. No. 6,015,891, and the references cited therein.

Those of skill in the art will recognize that, due to the degenerate nature of the genetic code, a variety of DNA compounds differing in their nucleotide sequences can be used to encode a given enzyme of the disclosure. The native DNA sequence encoding the biosynthetic enzymes described herein are referenced merely to illustrate an embodiment of the disclosure, and the disclosure includes DNA compounds of any sequence that encode the amino acid sequences of the polypeptides and proteins of the enzymes utilized in the methods of the disclosure. In similar fashion, a polypeptide can typically tolerate one or more amino acid substitutions, deletions, and insertions in its amino acid sequence without loss or significant loss of a desired activity. The disclosure includes such polypeptides with different amino acid sequences than the specific proteins described herein so long as they modified or variant polypeptides have the enzymatic anabolic or catabolic activity of the reference polypeptide. Furthermore, the amino acid sequences encoded by the DNA sequences shown herein merely illustrate embodiments of the disclosure.

In addition, homologs of enzymes useful for generating metabolites are encompassed by the microorganisms and methods provided herein. The term “homologs” used with respect to an original enzyme or gene of a first family or species refers to distinct enzymes or genes of a second family or species which are determined by functional, structural or genomic analyses to be an enzyme or gene of the second family or species which corresponds to the original enzyme or gene of the first family or species. Most often, homologs will have functional, structural or genomic similarities. Techniques are known by which homologs of an enzyme or gene can readily be cloned using genetic probes and PCR. Identity of cloned sequences as homolog can be confirmed using functional assays and/or by genomic mapping of the genes.

A protein has “homology” or is “homologous” to a second protein if the nucleic acid sequence that encodes the protein has a similar sequence to the nucleic acid sequence that encodes the second protein. Alternatively, a protein has homology to a second protein if the two proteins have “similar” amino acid sequences. (Thus, the term “homologous proteins” is defined to mean that the two proteins have similar amino acid sequences).

As used herein, two proteins (or a region of the proteins) are substantially homologous when the amino acid sequences have at least about 30%, 40%, 50% 60%, 65%, 70%, 75%, 80%, 85%, 90%, 91%, 92%, 93%, 94%, 95%, 96%, 97%, 98%, or 99% identity. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In one embodiment, the length of a reference sequence aligned for comparison purposes is at least 30%, typically at least 40%, more typically at least 50%, even more typically at least 60%, and even more typically at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences. Sequence for each of the genes and polypeptides/enzymes listed herein can be readily identified using databases available on the World-Wide-Web (see, e.g., http: (//)eecoli.kaist.ac.kr/main.html). In addition, the amino acid sequence and nucleic acid sequence can be readily compared for identity using commonly used algorithms in the art.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art (see, e.g., Pearson et al., 1994, hereby incorporated herein by reference).

The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Methionine (M), Alanine (A), Valine (V), and 6) Phenylalanine (F), Tyrosine (Y), Tryptophan (W).

Sequence homology for polypeptides, which is also referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild type protein and a mutein thereof. See, e.g., GCG Version 6.1.

A typical algorithm used comparing a molecule sequence to a database containing a large number of sequences from different organisms is the computer program BLAST (Altschul, 1990; Gish, 1993; Madden, 1996; Altschul, 1997; Zhang, 1997), especially blastp or tblastn (Altschul, 1997). Typical parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62.

When searching a database containing sequences from a large number of different organisms, it is typical to compare amino acid sequences. Database searching using amino acid sequences can be measured by algorithms other than blastp known in the art. For instance, polypeptide sequences can be compared using FASTA, a program in GCG Version 6.1. FASTA provides alignments and percent sequence identity of the regions of the best overlap between the query and search sequences (Pearson, 1990, hereby incorporated herein by reference). For example, percent sequence identity between amino acid sequences can be determined using FASTA with its default parameters (a word size of 2 and the PAM250 scoring matrix), as provided in GCG Version 6.1, hereby incorporated herein by reference.

The disclosure provides accession numbers for various genes, homologs and variants useful in the generation of recombinant microorganism described herein. It is to be understood that homologs and variants described herein are exemplary and nonlimiting. Additional homologs, variants and sequences are available to those of skill in the art using various databases including, for example, the National Center for Biotechnology Information (NCBI) access to which is available on the World-Wide-Web.

The disclosure also provides deposited microorganisms. The deposited microorganisms are exemplary only and, based upon the disclosure, one of ordinary skill in the art can modify additional parental organisms of different species or genotypes to arrive at a microorganism of the disclosure that can incorporate a C1 substrate into the cell’s mass.

The disclosure provides a recombinant microorganism designated Escherichi coli SM1 and having ATCC accession no. PTA-126783 as deposited with the ATCC on Jun. 19, 2020 (ATCC Patent Depository, 10801 University Boulevard, Manassas, Virginia 20110, U.S.A.). The disclosure includes cultures of microorganisms comprising a population of a microorganism of ATCC accession no. PTA-126783 including mixed cultures. Also provided are polynucleotide fragments derived from ATCC accession no. PTA-126783, which are useful in the preparation of a microorganism that can survive on methanol as a source of carbon. Also included are bioreactors comprising a population of the microorganism having ATCC accession no. PTA-126783. One of ordinary skill in the art, using the deposited microorganism, can readily determine the sequence of the deposited organism or fragments thereof encoding any of the genes and polynucleotides described herein, including locations of knockouts or gene disruptions. Moreover, the disclosure contemplates the use of the deposited microorganisms in the development of child-strains having improved activity and product production. For example, using the microorganism of the disclosure, one can engineer the microorganisms to use methanaol as a carbon source for the production of various chemicals and alcohols.

The synthetic methyltrophs of the disclosure including the deposited strains can be used in a bioreactor system for the processing of methane, formate or carbon dioxide, wherein the methane is converted to methanol upon which the recombinant microorganisms of the disclosure (i.e., the synthetic methylotrophs) can be cultured to produce more complex chemicals and/or alcohols.

The term “prokaryotes” is art recognized and refers to cells which contain no nucleus or other cell organelles. The prokaryotes are generally classified in one of two domains, the Bacteria and the Archaea. The definitive difference between organisms of the Archaea and Bacteria domains is based on fundamental differences in the nucleotide base sequence in the 16S ribosomal RNA.

The term “Archaea” refers to a categorization of organisms of the division Mendosicutes, typically found in unusual environments and distinguished from the rest of the procaryotes by several criteria, including the number of ribosomal proteins and the lack of muramic acid in cell walls. On the basis of ssrRNA analysis, the Archaea consist of two phylogenetically-distinct groups: Crenarchaeota and Euryarchaeota. On the basis of their physiology, the Archaea can be organized into three types: methanogens (prokaryotes that produce methane); extreme halophiles (prokaryotes that live at very high concentrations of salt ((NaCl)); and extreme (hyper) thermophilus (prokaryotes that live at very high temperatures). Besides the unifying archaeal features that distinguish them from Bacteria (i.e., no murein in cell wall, esterlinked membrane lipids, etc.), these prokaryotes exhibit unique structural or biochemical attributes which adapt them to their particular habitats. The Crenarchaeota consists mainly of hyperthermophilic sulfur-dependent prokaryotes and the Euryarchaeota contains the methanogens and extreme halophiles.

“Bacteria”, or “eubacteria”, refers to a domain of prokaryotic organisms. Bacteria include at least 11 distinct groups as follows: (1) Gram-positive (gram+) bacteria, of which there are two major subdivisions: (1) high G+C group (Actinomycetes, Mycobacteria, Micrococcus, others) (2) low G+C group (Bacillus, Clostridia, Lactobacillus, Staphylococci, Streptococci, Mycoplasmas); (2) Proteobacteria, e.g., Purple photosynthetic +non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; (11) Thermotoga and Thermosipho thermophiles.

“Gram-negative bacteria” include cocci, nonenteric rods, and enteric rods. The genera of Gram-negative bacteria include, for example, Neisseria, Spirillum, Pasteurella, Brucella, Yersinia, Francisella, Haemophilus, Bordetella, Escherichia, Salmonella, Shigella, Klebsiella, Proteus, Vibrio, Pseudomonas, Bacteroides, Acetobacter, Aerobacter, Agrobacterium, Azotobacter, Spirilla, Serratia, Vibrio, Rhizobium, Chlamydia, Rickettsia, Treponema, and Fusobacterium.

“Gram positive bacteria” include cocci, nonsporulating rods, and sporulating rods. The genera of gram positive bacteria include, for example, Actinomyces, Bacillus, Clostridium, Corynebacterium, Erysipelothrix, Lactobacillus, Listeria, Mycobacterium, Myxococcus, Nocardia, Staphylococcus, Streptococcus, and Streptomyces.

The term “recombinant microorganism” and “recombinant host cell” are used interchangeably herein and refer to microorganisms that have been genetically modified to express or over-express endogenous polynucleotides, or to express non-endogenous sequences, such as those included in a vector, or which have a reduction in expression of an endogenous gene. The polynucleotide generally encodes a target enzyme involved in a metabolic pathway for producing a desired metabolite as described above. Accordingly, recombinant microorganisms described herein have been genetically engineered to express or over-express target enzymes not previously expressed or over-expressed by a parental microorganism. It is understood that the terms “recombinant microorganism” and “recombinant host cell” refer not only to the particular recombinant microorganism but to the progeny or potential progeny of such a microorganism.

A “parental microorganism” refers to a cell used to generate a recombinant microorganism. The term “parental microorganism” describes a cell that occurs in nature, i.e. a “wild-type” cell that has not been genetically modified. The term “parental microorganism” also describes a cell that has been genetically modified. For example, a wild-type microorganism can be genetically modified to express or over express a first target enzyme. This microorganism can act as a parental microorganism in the generation of a microorganism modified to express or over-express a second target enzyme etc. Accordingly, a parental microorganism functions as a reference cell for successive genetic modification events. Each modification event can be accomplished by introducing a nucleic acid molecule in to the reference cell. The introduction facilitates the expression or over-expression of a target enzyme. It is understood that the term “facilitates” encompasses the activation of endogenous polynucleotides encoding a target enzyme through genetic modification of e.g., a promoter sequence in a parental microorganism. It is further understood that the term “facilitates” encompasses the introduction of exogenous polynucleotides encoding a target enzyme in to a parental microorganism.

A “protein” or “polypeptide”, which terms are used interchangeably herein, comprises one or more chains of chemical building blocks called amino acids that are linked together by chemical bonds called peptide bonds. An “enzyme” means any substance, composed wholly or largely of protein, that catalyzes or promotes, more or less specifically, one or more chemical or biochemical reactions. The term “enzyme” can also refer to a catalytic polynucleotide (e.g., RNA or DNA). A “native” or “wild-type” protein, enzyme, polynucleotide, gene, or cell, means a protein, enzyme, polynucleotide, gene, or cell that occurs in nature.

It is understood that the polynucleotides described above include “genes” and that the nucleic acid molecules described above include “vectors” or “plasmids.” For example, a polynucleotide encoding a methanol dehydrogenase can be encoded by an medh gene or homolog thereof. Accordingly, the term “gene”, also called a “structural gene” refers to a polynucleotide that codes for a particular sequence of amino acids, which comprise all or part of one or more proteins or enzymes, and may include regulatory (non-transcribed) DNA sequences, such as promoter sequences, which determine for example the conditions under which the gene is expressed. The transcribed region of the gene may include untranslated regions, including introns, 5′-untranslated region (UTR), and 3′-UTR, as well as the coding sequence. The term “nucleic acid” or “recombinant nucleic acid” refers to polynucleotides such as deoxyribonucleic acid (DNA), and, where appropriate, ribonucleic acid (RNA). The term “expression” with respect to a gene sequence refers to transcription of the gene and, as appropriate, translation of the resulting mRNA transcript to a protein. Thus, as will be clear from the context, expression of a protein results from transcription and translation of the open reading frame sequence.

The term “operon” refers to two or more genes which are transcribed as a single transcriptional unit from a common promoter. In some embodiments, the genes comprising the operon are contiguous genes. It is understood that transcription of an entire operon can be modified (i.e., increased, decreased, or eliminated) by modifying the common promoter. Alternatively, any gene or combination of genes in an operon can be modified to alter the function or activity of the encoded polypeptide. The modification can result in an increase in the activity of the encoded polypeptide. Further, the modification can impart new activities on the encoded polypeptide. Exemplary new activities include the use of alternative substrates and/or the ability to function in alternative environmental conditions.

A “vector” is any means by which a nucleic acid can be propagated and/or transferred between organisms, cells, or cellular components. Vectors include viruses, bacteriophage, pro-viruses, plasmids, phagemids, transposons, and artificial chromosomes such as YACs (yeast artificial chromosomes), BACs (bacterial artificial chromosomes), and PLACs (plant artificial chromosomes), and the like, that are “episomes,” that is, that replicate autonomously or can integrate into a chromosome of a host cell. A vector can also be a naked RNA polynucleotide, a naked DNA polynucleotide, a polynucleotide composed of both DNA and RNA within the same strand, a poly-lysine -conjugated DNA or RNA, a peptide-conjugated DNA or RNA, a liposome-conjugated DNA, or the like, that are not episomal in nature, or it can be an organism which comprises one or more of the above polynucleotide constructs such as an agrobacterium or a bacterium. The disclosure provides a number of vectors (plasmids) in Table 5.

“Transformation” refers to the process by which a vector is introduced into a host cell. Transformation (or transduction, or transfection), can be achieved by any one of a number of means including electroporation, microinjection, biolistics (or particle bombardment-mediated delivery), or agrobacterium mediated transformation.

The disclosure provides nucleic acid molecules in the form of recombinant DNA expression vectors or plasmids, as described in more detail below, that encode one or more target enzymes. Generally, such vectors can either replicate in the cytoplasm of the host microorganism or integrate into the chromosomal DNA of the host microorganism. In either case, the vector can be a stable vector (i.e., the vector remains present over many cell divisions, even if only with selective pressure) or a transient vector (i.e., the vector is gradually lost by host microorganisms with increasing numbers of cell divisions). The disclosure provides DNA molecules in isolated (i.e., not pure, but existing in a preparation in an abundance and/or concentration not found in nature) and purified (i.e., substantially free of contaminating materials or substantially free of materials with which the corresponding DNA would be found in nature) forms.

The term expression vector refers to a nucleic acid that can be introduced into a host microorganism or cell-free transcription and translation system. An expression vector can be maintained permanently or transiently in a microorganism, whether as part of the chromosomal or other DNA in the microorganism or in any cellular compartment, such as a replicating vector in the cytoplasm. An expression vector also comprises a promoter that drives expression of an RNA, which typically is translated into a polypeptide in the microorganism or cell extract. For efficient translation of RNA into protein, the expression vector also typically contains a ribosome-binding site sequence positioned upstream of the start codon of the coding sequence of the gene to be expressed. Other elements, such as enhancers, secretion signal sequences, transcription termination sequences, and one or more marker genes by which host microorganisms containing the vector can be identified and/or selected, may also be present in an expression vector. Selectable markers, i.e., genes that confer antibiotic resistance or sensitivity, are used and confer a selectable phenotype on transformed cells when the cells are grown in an appropriate selective medium.

The various components of an expression vector can vary widely, depending on the intended use of the vector and the host cell(s) in which the vector is intended to replicate or drive expression. Expression vector components suitable for the expression of genes and maintenance of vectors in E. coli, yeast, Streptomyces, and other commonly used cells are widely known and commercially available. For example, suitable promoters for inclusion in the expression vectors of the disclosure include those that function in eukaryotic or prokaryotic host microorganisms. Promoters can comprise regulatory sequences that allow for regulation of expression relative to the growth of the host microorganism or that cause the expression of a gene to be turned on or off in response to a chemical or physical stimulus. For E. coli and certain other bacterial host cells, promoters derived from genes for biosynthetic enzymes, antibiotic-resistance conferring enzymes, and phage proteins can be used and include, for example, the galactose, lactose (lac), maltose, tryptophan (trp), beta-lactamase (bla), bacteriophage lambda PL, and T5 promoters. In addition, synthetic promoters, such as the tac promoter (U.S. Pat. No. 4,551,433), can also be used. For E. coli expression vectors, it is useful to include an E. coli origin of replication, such as from pUC, p1P, p1, and pBR.

Thus, recombinant expression vectors contain at least one expression system, which, in turn, is composed of at least a portion of PKS and/or other biosynthetic gene coding sequences operably linked to a promoter and optionally termination sequences that operate to effect expression of the coding sequence in compatible host cells. The host cells are modified by transformation with the recombinant DNA expression vectors of the disclosure to contain the expression system sequences either as extrachromosomal elements or integrated into the chromosome.

A nucleic acid of the disclosure can be amplified using cDNA, mRNA or alternatively, genomic DNA, as a template and appropriate oligonucleotide primers according to standard PCR amplification techniques and those procedures described in the Examples section below. The nucleic acid so amplified can be cloned into an appropriate vector and characterized by DNA sequence analysis. Furthermore, oligonucleotides corresponding to nucleotide sequences can be prepared by standard synthetic techniques, e.g., using an automated DNA synthesizer.

It is also understood that an isolated nucleic acid molecule encoding a polypeptide homologous to the enzymes described herein can be created by introducing one or more nucleotide substitutions, additions or deletions into the nucleotide sequence encoding the particular polypeptide, such that one or more amino acid substitutions, additions or deletions are introduced into the encoded protein. Mutations can be introduced into the polynucleotide by standard techniques, such as site-directed mutagenesis and PCR-mediated mutagenesis. In contrast to those positions where it may be desirable to make a non-conservative amino acid substitutions (see above), in some positions it is preferable to make conservative amino acid substitutions. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), betabranched side chains (e.g., threonine, valine, isoleucine) and aromatic side chains (e.g., tyrosine, phenylalanine, tryptophan, histidine).

As previously discussed, general texts which describe molecular biological techniques useful herein, including the use of vectors, promoters and many other relevant topics, include Berger and Kimmel, Guide to Molecular Cloning Techniques, Methods in Enzymology Volume 152, (Academic Press, Inc., San Diego, Calif.) (“Berger”); Sambrook et al., Molecular Cloning—A Laboratory Manual, 2d ed., Vol. 1-3, Cold Spring Harbor Laboratory, Cold Spring Harbor, N.Y., 1989 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 1999) (“Ausubel”). Examples of protocols sufficient to direct persons of skill through in vitro amplification methods, including the polymerase chain reaction (PCR), the ligase chain reaction (LCR), Qβ-replicase amplification and other RNA polymerase mediated techniques (e.g., NASBA), e.g., for the production of the homologous nucleic acids of the disclosure are found in Berger, Sambrook, and Ausubel, as well as in Mullis et al. (1987) U.S. Pat. No. 4,683,202; Innis et al., eds. (1990) PCR Protocols: A Guide to Methods and Applications (Academic Press Inc. San Diego, Calif.) (“Innis”); Arnheim & Levinson (Oct. 1, 1990) C&EN 36-47; The Journal Of NIH Research (1991) 3: 81-94; Kwoh et al. (1989) Proc. Natl. Acad. Sci. USA 86: 1173; Guatelli et al. (1990) Proc. Nat′l. Acad. Sci. USA 87: 1874; Lomell et al. (1989) J. Clin. Chem 35: 1826; Landegren et al. (1988) Science 241: 1077-1080; Van Brunt (1990) Biotechnology 8: 291-294; Wu and Wallace (1989) Gene 4:560; Barringer et al. (1990) Gene 89:117; and Sooknanan and Malek (1995) Biotechnology 13: 563-564. Improved methods for cloning in vitro amplified nucleic acids are described in Wallace et al., U.S. Pat. No. 5,426,039. Improved methods for amplifying large nucleic acids by PCR are summarized in Cheng et al. (1994) Nature 369: 684-685 and the references cited therein, in which PCR amplicons of up to 40 kb are generated. One of skill will appreciate that essentially any RNA can be converted into a double stranded DNA suitable for restriction digestion, PCR expansion and sequencing using reverse transcriptase and a polymerase. See, e.g., Ausubel, Sambrook and Berger, all supra.

Appropriate culture conditions are conditions of culture medium pH, ionic strength, nutritive content, etc.; temperature; oxygen/CO₂/nitrogen content; humidity; and other culture conditions that permit production of the compound by the host microorganism, i.e., by the metabolic action of the microorganism. Appropriate culture conditions are well known for microorganisms that can serve as host cells.

The disclosure is illustrated in the following examples, which are provided by way of illustration and are not intended to be limiting.

Exemplary microorganisms of the disclosure were deposited on Jun. 19, 2020 with the American Type Culture Collection (ATCC), 10801 University Boulevard, Manassas, Virginia 20110, U.S.A., as ATCC Number PTA-126783 (designation Escherichi coli SM1) under the Budapest Treaty. This deposit will be maintained at an authorized depository and replaced in the event of mutation, nonviability or destruction for a period of at least five years after the most recent request for release of a sample was received by the depository, for a period of at least thirty years after the date of the deposit, or during the enforceable life of the related patent, whichever period is longest. All restrictions on the availability to the public of these cell lines will be irrevocably removed upon the issuance of a patent from the application.

### EXAMPLES

Escherichia Coli. E. coli K-12 BW25113 was used as the experimental model.

Media and growth conditions. All strains were grown in at 37° C. 250 rpm in a New Brunswick Scientific Innova 44, unless specified otherwise. LB (Becton Dickinson) was used for cloning purposes and the priority media when rich media was required. Antibiotics were used when required, at a final concentration of 100 mg/L for carbenicillin, 30 mg/L for kanamycin, 50 mg/L for chloramphenicol, or 250 mg/L for spectinomycin. Hi Def azure media (HDA, Teknova) was used as a preliminary-stage medium for adaptive evolution with limited nutrients. MOPS EZ buffer (MOPS, Teknova) was modified and utilized as a minimal medium, which consisted of 40 mM MOPS, 50 mM NaCl, 9.5 mM NH₄Cl, 0.525 mM MgCl₂, 4 mM tricine, 1.32 mM K₂PO₄, 0.276 mM K₂SO₄, 0.01 mM FeSO₄, 0.5 µM CaCl₂, 40 nM H₃BO₃, 8.08 nM MnCl₂, 3.02 nM CoCl₂, 0.962 nM CuSO₄, 0.974 nM ZnSO₄, and 0.292 nM (NH₄) ₂MoO₄. OD₆₀₀ was monitored by a G30 spectrometer (Thermo Scientific).

Strain construction and adaptive evolution of the methanol auxotrophy strain. Strains used in this study are summarized in Table 2. The initial auxotrophy strain, CFC381.0 was constructed from a ΔrpiA strain included in Keio collection (Baba et al., 2006), followed by kanamycin cassette removal with a pCP20 plasmid encoding the FLP recombinase (Cherepanov and Wackernagel, 1995). Subsequently, rpiB removal and insertion of two operons, PLlacO1::medh-hps(Mb)-phi in the SS3 site (between ompW and yciE) (Bassalo et al., 2016) and PLlacO1::medh-tkt(Mb)-tal(Kp)-hps(Mb)-phi in the nupG site, were performed by a modified Crispr/Cas9 system from Jiang et al. (Jiang et al., 2015). pCas9-transformed strains were grown overnight, and reinoculated with an initial OD=0.1 and grown for 4 hrs in a 30° C. shaker with LB and 100 mM arabinose. The strains are then electroporated with the pTarget plasmid and plated on a LB plate at 30° C. overnight. Successful gene editing targets were confirmed with colony PCR. The pTarget plasmid was then removed by growing cell on LB with 0.1 mM IPTG, while the pCas9 was almost ultimately removed by growing the variant in LB 37° C. and extensive screening. All E. coli strains were grown in a 3 ml PP tube with the cap sealed to prevent evaporation and transferred to the next passage when OD₆₀₀ exceeded 1.2, or reached a stationary phase. Strains are typically inoculated at an initial OD₆₀₀ at 0.05 to 0.2. Accordingly, the population size at the bottleneck between transfers are approximately ~1.5-6x10⁸ cells. Thus, effectively about 3-4 generations elapsed per passage. The unevolved ΔrpiAB strain CFC381 was first inoculated in Terrific Broth (TB, Sigma) with 20 mM ribose and 20 mM xylose. Strains grew to saturation in two days and were then passed to HDA and MOPS separately, both with 400 mM methanol and 20 mM xylose induced by 1 mM IPTG (media named as HMX and MMX, respectively). From Passage 2 to Passage 10, cells were passed from HMX to HMX. Beginning from Passage 11, cells were passed into MMX up until Passage 21 (CFC381.20), where the strain was subjected to further gene modifications after incorporating results suggested from theoretical calculations (See EMRA details).

Strain construction and adaptive evolution of methanol growth strain. After methanol auxotroph was achieved from adaptive evolution, further knockout of pfkA and replacement of gapA with gapC were implemented by Crispr/Cas9 considering EMRA results. The Crispr protocol is identical to the one in the previous section. A plasmid pFC139 consisting rpiA with an RBS library was transformed into the strain by electroporation. Adaptive evolution was carried out by mixing a ratio of HDA and MOPS with 400 mM of methanol. In addition, a vitamin mix was added regardless of the ratio of MOPS and HDA medium, where the following final concentration is reached: 40.94 µM nicotinamide, 14.82 µM thiamine hydrochloride, 13.29 µM riboflavin, 10.49 µM calcium pantothenate, 8.19 µM biotin, 4.53 µM folic acid, 0.07 µM vitamin B12. Moreover, 10 mM of NaNO₃ was incorporated and 1 mM of IPTG was used for induction at the point of inoculation. The evolution began with 100:0 of the HDA: MOPS media (After adding methanol and all supplement vitamins, the actual ratio of HDA medium was diluted to 95.2%). At passage 2 (CFC526.2), HDA: MOPS was adjusted to 50%. From passage 3 (CFC526.3) to passage 16 (CFC526.16), HDA was reduced to 30% HDA ratio was further reduced to 20% and 10% from passage 17 (CFC526.17) to 19 (CFC526.19), and passage 19 (CFC526.19) to 20 (CFC526.20), respectively. Finally, HDA was completely eliminated where MOPS was used on Passage 21, which was renamed to CFC680.1. CFC680.1 was then further evolved on solely MOPS and 400 mM methanol for 31 passages until CFC680.31. CFC688.2 was simultaneously grown from CFC680.1 with MOPS and 400 mM methanol without nitrate, and then evolved for 30 passages until CFC 688.32. Further, at CFC526.20, a slower HDA approach was done. Specifically, 10% and 5% HDA supplement was provided until passage 21 (CFC526.21) and passage 22 (CFC526.22) respectively. HDA was completely omitted at passage 23 (CFC526.23). CFC526.23 was then evolved for 30 passages until CFC526.53.

A final single colony of strain SM1 was obtained by first streaking out CFC526.53 on a MOPS plus 400 mM methanol agar plate. The single colony was then inoculated into MOPS plus 400 mM methanol liquid culture again, followed by streaking out on a LB plate in anaerobic conditions. The SM1 was finally retrieved by growing single colonies in LB with colony-PCR confirmation. The other single colony strain BB1 was simply isolated by growing CFC526.53 in LB liquid and LB plate.

Plasmid Construction. All plasmids are summarized in the Resources Table. All of the plasmids were constructed by Gibson Assembly with the NEBuilder kit (New England Biolabs) while DNA fragments were amplified by KODone (Toyobo). E. coli DH5alpha was used as the cloning host.

The final SM1 strain is grown in the 1x MOPS EZ media (10X stock from Catalog No. M2101, Teknova) along with 400 mM MeOH, the previously mentioned vitamin mix, 1 mM IPTG and 50 mg/L chloramphenicol. Note that the chloramphenicol was dissolved in pure methanol as well, and was added to the media by a 1000x stock.

RESOURCES TABLE

| REAGENT or RESOURCE | SOURCE | IDENTIFIER |
|---|---|---|
| Bacterial and Virus Strains | | |
| E. coli strain BW25113 | Coli Genetic Stock Center (CGSC) | CGSC #: 7636 |
| E. coli strain BW25113 ΔrpiA | CGSC | CGSC #: 11414 |
| E. coli strain Dh5alpha | CGSC | CGSC #: 14231 |
| Chemicals, Peptides, and Recombinant Proteins | | |
| Media | | |
| Luria-Bertani (LB) | BD | BD 244610 |
| Bacto Agar | BD | BD 214010 |
| Hi Def azure media (HDA) | Teknova | Cat. #3H5000 |
| MOPS EZ buffer | Teknova | Cat. #M2101 |
| Terrific Broth (TB) | Sigma-Aldrich | Cat. #T0918 |
| Antibiotics | | |
| Carbenicillin disodium | AKsci | Cat. # C435 |
| Kanamycin Sulfate | Amresco | Cat. # 97062-956 |
| Chloramphenicol | Sigma-Aldrich | Cat. # C0378 |
| Spectinomycin | Sigma-Aldrich | Cat. # S4014 |
| D-(-)-Ribose | Sigma-Aldrich | Cat. #R7500 |
| D-Xylose | Sigma-Aldrich | Cat. #X1500 |
| Methanol | Sigma-Aldrich | Cat. #646377 |
| Isopropyl β-D-1-thiogalactopyranoside (IPTG) | Amresco | Cat. #0487-10G |
| Nicotinamide | Sigma-Aldrich | Cat. #N0636 |
| Thiamine hydrochloride | Sigma-Aldrich | Cat. #T1270 |
| Riboflavin | Sigma-Aldrich | Cat. #R9504 |
| Calcium pantothenate | Sigma-Aldrich | Cat. #C8731 |
| Biotin | Sigma-Aldrich | Cat. #B4639 |
| Folic acid | Sigma-Aldrich | Cat. #8758 |
| Vitamin B12 | Sigma-Aldrich | Cat. #V6629 |
| Sodium nitrate | Alfa Aesar | Cat. #AF-14493 |
| Methanol-¹³C, | Sigma-Aldrich | Cat. #277177 |
| Sodium acetate-¹³C₂ | Sigma-Aldrich | Cat. #282014 |
| Sodium formate-¹³C | Sigma-Aldrich | Cat. #279412 |
| Lysozyme | Bioshop | Cat. #LYS702 |
| DNAzol reagent | ThermoFisher | Cat. #10503027 |
| Ethanol | Fisher | Cat. #AC615090010 |
| Urea | Sigma-Aldrich | Cat. #15604 |
| Sodium dodecyl sulfate (SDS) | Amresco | Cat. #AM-0227 |
| Sodium hydroxide | Amresco | Cat. #AM-E584 |
| Tris | VWR | Cat. #PT-0826 |
| Hydrochloric Acid | J.T.Baker | Cat #9673-00 |
| L-(+)-Arabinose | Sigma-Aldrich | Cat. #A3256 |
| Miniprep kit | Qiagen | Cat. #27106 |
| Puregene kit | Qiagen | Cat. #69506 |
| NEBuilder kit | New England BioLabs | NEB #C2987 |
| Pierce™ silver staining kit | Thermo Scientific | Cat. #24612 |
| Pierce™Coomassie Plus (Bradford) Assay | Thermo Scientific | Cat. #23236 |
| KAPA Hyper Prep Kit | Roche | Cat. 07962363001 |
| Ligation Sequencing Kit | Oxford Nanopore technologies | Cat. SQK-LSK109 |
| RNeasy Plus mini kit | Qiagen | Cat. #74136 |
| QuantiNova reverse transcription kit | Qiagen | Cat. #205413 |
| QuantiNova SYBR green RT-PCR kit | Qiagen | Cat. #208152 |
| CFC381 | This study | See Table 3 |
| CFC381.20 | This study | See Table 3 |
| CFC526.0 | This study | See Table 3 |
| CFC526.53 | This study | See Table 3 |
| CFC680.1 | This study | See Table 3 |
| CFC680.32 | This study | See Table 3 |
| CFC688.1 | This study | See Table 3 |
| CFC688.32 | This study | See Table 3 |
| SM1 | This study | See Table 3 |
| BB1 | This study | See Table 3 |
| See Table 4 for primers used | Purigo | N/A |
| See Table 5 for all plasmids used and constructed | See Table 5 | See Table 5 |
| Microsoft Excel | Microsoft | www.microsoft.com |
| R software | R Core Team | www.r-project.org |
| CLC Genomics Workbench 20 | Qiagen | https[://]digitalinsigh ts.qiagen.com/prod ucts-overview/discovery-insights-portfolio/analysis-and-visualization/qiagen -clc-genomics-workbench/ |
| Geneious 2020 | Geneious | [www.]geneious.co m/ |
| Matlab 2019b | Matlab | mathworks.com |
| MUMmer4 | MUMmer4 | [www.]mummer4.git hub.io/ |

TABLE 3

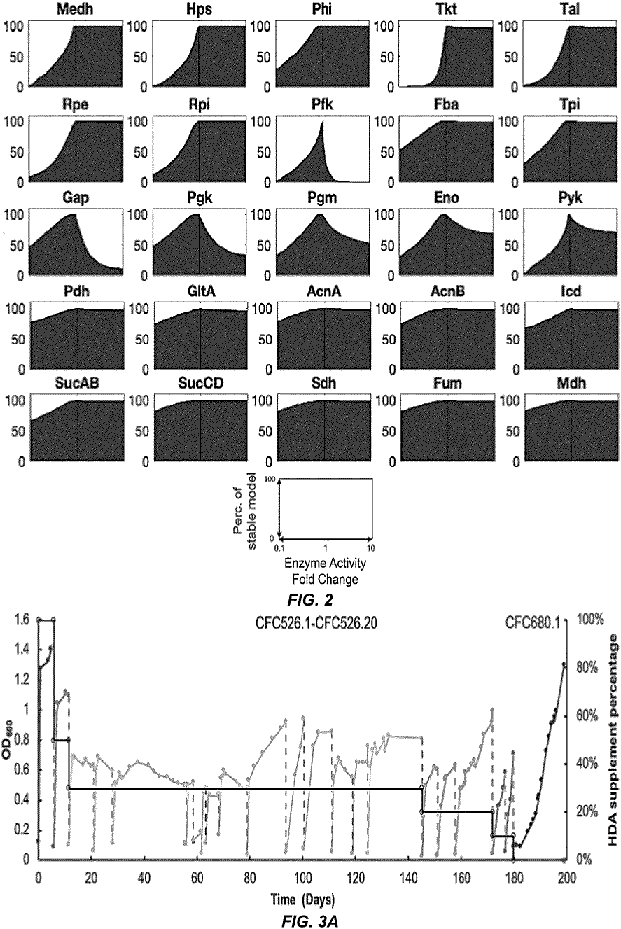

Strain list

| | | |
|---|---|---|
| Name | Genotype | Description |
| Dh5alpha | F- endA1 glnV44 thi-1 recA1 relA1 gyrA96 deoR nupG purB20 φ80d/acZΔM15 Δ(lacZYA-argF)U169, hsdR17(r_{K}⁻m_{K}⁺), A- | Wild-type used for plasmid construction. |
| BW25113 | F- LAM- rmB3 ΔlacZ4787 hsdR514 Δ(araBAD)567 Δ(rhaBAD)568 rph-1 | Wild-type used for establishing synthetic methanol growth strain. |
| CFC381 | BW25113 ΔrpiA::FRT ΔrpiB ΔnupG::P_{L}lacO₁::medh/tkt(Mb)/tal(Kp)/hps(Mb)/phi SS3(intergenic site)::P_{L}lacO₁::medh/hps(Bm)/phi | Initial strain to evolve for methanol auxotrophy |
| CFC381.20 | Refer to FIG. 2C | CFC381 evolved to grow in methanol and xylose after 20 passages. A methanol auxotroph. |
| CFC526.0 | CFC381.20 ΔptkA ΔgapA::gapC with pFC139 | Initial strain to evolve for synthetic methylotrophy |
| CFC526.53 | Undetermined (Mixed culture) | Evolved from CFC526.0 to grow in methanol plus nitrate, with slower nutrient reduction. |
| CFC680.1 | Undetermined (Mixed culture) | Evolved from CFC526.0 to grow in methanol plus nitrate with faster nutrient reduction. |
| CFC680.32 | Undetermined (Mixed culture) | CFC680.1 evolved to grow in methanol after 32 passages. |
| CFC688.1 | Undetermined (Mixed culture) | CFC680.1 grown in methanol without nitrate |
| CFC688.32 | Undetermined (Mixed culture) | CFC688.1 to grow in methanol without nitrate after 32 passages. |
| SM1 | Refer to Table 1, with pFC139C | Isolated single colony from CFC526.53 that can grow in methanol without nitrate or vitamin |
| BB1 | Refer to Table 1, with pFC139B | Isolated single colony from CFC526.53 that cannot grow in methanol |

TABLE 4

Primer list.

| | |
|---|---|
| Description | Sequence (SEQ ID NO) |
| rpiA knockout validation forward primer | 5′- CGCCTTCTACCAGCAGAAAC -3′ (SEQ ID NO:23) |
| rpiA knockout validation reverse primer | 5′- CCCAGACCGTTGTATGCTTT -3′(SEQ ID NO:24) |
| rpiB knockout validation forward primer | 5′- GGAAGCGCTGAATCAAACTC -3′(SEQ ID NO:25) |
| rpi8 knockout validation reverse primer | 5′- GCTCTTCATCCTCCAGTTGC -3′(SEQ ID NO:26) |
| nupG knock-in validation forward primer | 5′- ATATGCCATTTGCCACACCA -3′(SEQ ID NO:27) |
| nupG knock-in validation reverse primer | 5′- CTTATATTCGCGGTGACGTG -3′(SEQ ID NO:28) |
| SS3 site knock-in validation forward primer | 5′- TGTTAATTAGCGGGCAATTGTACC -3′(SEQ ID NO:29) |
| SS3 site knock-in validation reverse primer | 5′- GATACCTACAGCGCAGAAAAACAA -3′(SEQ ID NO:30) |
| gapA knockout/gapC knock-in validation forward primer | 5′- TGCTTCGATATTATGGCGGGCTT -3′(SEQ ID NO:31) |
| gapA knockout/gapC knock-in validation reverse primer | 5′- GCCAGATGTGCAGGTTTCTCTTT -3′(SEQ ID NO:32) |
| pfkA knockout validation forward primer | 5′- ATCAATCTTATGGACGGCTGGTC -3′(SEQ ID NO:33) |
| pfkA knockout validation reverse primer | 5′- TGCTGATCTGATCGAACGTACCG -3′(SEQ ID NO:34) |
| frmA frameshift validation forward primer | 5′- TATTTTGCCAGCCGCCAAAG -3′(SEQ ID NO:35) |
| frmA frameshift validation reverse primer | 5′- CGAAATGACTGCTACAGCCG -3′(SEQ ID NO:36) |
| pgi deletion validation forward primer | 5′- GAAGTCAACGCGGTGCTG -3′(SEQ ID NO:37) |
| pgi deletion validation reverse primer | 5′- CCCTGGTGGATCAGCTGG -3′(SEQ ID NO:38) |
| pFC139 plasmid variation validation forward primer | 5′- ATCCTACTGCTTTTTTCAATTCATC -3′(SEQ ID NO:39) |
| pFC139 plasmid variation validation reverse primer | 5′- CAAGGGTGAACACTATCCCA -3′(SEQ ID NO:40) |

TABLE 5

Plasmid list.

| | | |
|---|---|---|
| Name | Description | Reference |
| pCas9 | Plasmid for Cripsr-Cas9 with lambda-red recombinase system | Yu Jiang, et al. |
| pFC98 | pTarget for Crispr knockout of ΔrpiB Spec^{R} | This disclosure |
| pFC99 | pTarget for Crispr editing of ΔSS3::P_{L}lacO₁::medh/hps(Mb)/phi Spec^{R} | This disclosure |
| pCT309 | pTarget for Crispr editing of ΔnupG::P_{L}lacO₁::medh/tkt(Mb)/tal(Kp)/hps(Mb)/phi Spec^{R} | This disclosure |
| pFC139 | PL/acO₁::RBS library::rpiA p15A ori Cm^{R} Selected from pFC139 library with a specific RBS | This disclosure |
| pFC139A | 5′-GACTAAAAACATTCGGAGGCTTAAGCAGTCATCGT-3′ (SEQ ID NO:45) | This disclosure |
| pFC139B | Same as pFC139, but with IS2 sequence between p15A and cat, Triplicated UTR before rpiA | This disclosure |
| pFC139C | Same as pFC139A, but with an additional IS2 between cat and rpiA | This disclosure |
| pFC144 | pTarget for Crispr knockout of ΔpfkA Spec^{R} | This disclosure |
| pFC149 | pTarget for Crispr editing of ΔgapA::gapC Spec^{R} | This disclosure |
| pCY96 | Same as pBAC (oriV ori Amp^{R}), empty plasmid | This disclosure |
| pCY153 | pBAC::Native BW25113 pfkA operon (4,096,980-4,098,472) | This disclosure |
| pCY154 | pBAC::Native BW25113 frmA operon (373,388-375,577) | This disclosure |
| pCY156 | pBAC::Native BW25113 gapA operon (1,856,478-1,858,053) | This disclosure |
| pCY161 | pBAC::gapA operon (pCY156), pfkA operon (pCY153) | This disclosure |

Robustness of RuMP-EMP-TCA cycle by EMRA. EMRA is a calculation method developed to determine the likelihood of perturbations in enzyme expression and kinetics that cause instability of the steady state. After pre-setting a reference steady state for the entire pathway, a total of 100 parameter sets were then generated and perturbed randomly from 0.1-fold to 10-fold for each enzyme. Results were reported as an indication of the robustness of the system, where Y_{R,M} refers to the ratio of the 100 parameter sets that are robust at each point.

¹³C labelling experiment. Qualitative analysis of ¹³C labelled acetate and formate was conducted by an Agilent Technologies 7890 gas chromatography along with a 5977B mass spectrometer. Samples were prepared by aliquoting the supernatant of the culture after centrifugation at 15000 rpm for 3 minutes. 0.5 ul of sample was injected into the GC. A DB-FFAP column (Agilent Technologies, 0.32 mm × 30 m × 0.25 µm) was utilized along with constant pressure of 7.0633 psi helium gas supply. The thermal cycle was carried out with an initial temperature of 40° C. for 2 minutes, followed by a ramp rate of 10° C./min to 60° C. and 100° C./min to 240° C. along with a final 2-minute hold.

Cell viability Test. The cell viability assay was done by using LIVE/DEAD BacLight Bacterial Viability Kit (Thermofisher Scientific, USA) following its protocol. The fluorescence of cells was then detected by a 2018 Attune NxT Flow Cytometer (Thermofisher Scientific, USA). The Blue laser (Excitation Wavelength 488 nm) and BL1 filter (Emission filter 530±30 nm) was selected for SYTO-9 detection, while the yellow laser (Excitation Wavelength 561 nm) and YL2 filter (Emission filter 620±15 nm) was used for propidium iodide detection.

Isolation of DPC complexes. The extraction protocol was modified from that reported by Qiu et al. (Qiu and Wang, 2009b) and Barker et al. (Barker et al., 2005). 2 ml of E. coli (CFC526.41 and CFC680.24) was first pelleted by centrifugation at 5000 g for 5 minutes, and then resuspended in 100 µl of 10 mM MOPS buffer containing 2.0 mg/ml of lysozyme, with incubation of 30 minutes at 37° C. 500 µl of DNAzol reagent was then added and mixed for 5 minutes, followed by centrifugation at 12000 rpm for 10 minutes. The supernatant was then transferred into a new tube where 300 µl ice cold 100% ethanol was added for DNA precipitation. Samples were then stored at -80° C. for at least 1 hr. Subsequently, after removal of supernatant by centrifugation at 12000 rpm for 5 minutes, the DNA pellet was re-dissolved in 190 µl of 8 mM NaOH. Subsequently, 10 µl of 1 M Tris-HCl, pH 7.4 was added while urea and SDS were added as well to a final concentration of 8 M & 2% w/v respectively for protein denaturing and disassociation of non-specific binding protein to DNA. The entire mixture was gently shaken at 37° C. for 30 minutes. Protein was then salted out by adding equal volume of 5 M NaCl and subjected to gentle shaking at 37° C. for 30 minutes. After centrifugation at 12000 rpm, 20 minutes, the supernatant was transferred to an Amicon Ultra-4 mL Centrifugal Filters with a 3 kDa cutoff (Millipore) and washed with 10 mM of Tris pH 7.4 thrice to a final dilution factor of 10000. When the volume was finally concentrated to 450 µl, 50 µl of 3 M potassium acetate and 1 ml of ice-cold 100% ethanol were added and stored once again at -80° C. for 1 hr. After centrifugation at 12000 rpm, 20 minutes, 4° C., the DNA pellet was retained and washed with 1 ml 70% ethanol. The pellet was then dissolved with 10 mM Tris-HCl, typically 100 µl. The DNA was then quantified by 260 nm Nanodrop (Thermo).

Transmission electron microscopy. Purified DPC complex at a scale around 500 ng of DNA was mounted on an activated 300-mesh copper grid coated with carbon-stabilized formvar (Ted Pella) for 1 minute at room temperature. Following liquid removal by filter paper blotting, samples were stained with 2.5% uranyl acetate for 1 minute. After excess stain removal, the samples were air dried at room temperature. Transmission electron microscopy was performed with a Tecnai G2 Spirit Bio TWIN (FEI Co.), while images were recorded with a K3 Base IS CCD camera (Gatan) at a magnifying ratio of 2700x to 15000x.

Purification of DPC protein portion. Decrosslinking of the DNA was done by incubation at 70° C. for 1 hour, followed by a DNAase I (NEB) and S1 nuclease (Thermo) treatment, 1 ul each. The small digested DNA fragment was then removed by using an Amicon Ultra-0.5 mL Centrifugal Filters with a 3 kDa cutoff and the final volume was reduced to 50 µl. Sample concentration was then estimated by 280 nm Nanodrop. SDS-PAGE was run for quick analysis with a 12% precast gel (Biorad), while staining was done with a Pierce silver staining kit (Thermo Scientific).

Protein Sample preparation for quantitative proteomics and LC-MS/MS analysis. Proteins were denatured by adding urea to a final concentration of 4 M, followed by reduction with 10 mM dithioerythritol at 37° C. for 45 minutes, and cysteines alkylation with 25 mM iodoacetamide at room temperature in the dark for 1 hour. Protein samples were digested overnight at 37° C. using LysC protease and trypsin at an enzyme-to-substrate ratio of 1:50 (w/w). After tryptic digestion, the peptides were desalted directly by C18 StageTip. Samples were performed on EASY-nLCTM 1200 system connected to a Thermo Scientific Orbitrap Fusion Lumos Tribrid Mass Spectrometer (Thermo Fisher Scientific, Bremen, Germany). Data analysis was performed using SEQUEST HT algorithm integrated in the Proteome Discoverer 2.4 (Thermo Finnigan). MS/MS scans were matched against an E. coli K12 database (UniProtKB/Swiss-Prot 2019_10 Release).

The data was analyzed by first normalizing the abundance of the internal standard DNase to the same value within different time points of the same sample. Then the normalized abundance of each sample was divided by their DNA concentration. Last, the heat map was plotted by listing out the top 100 hits of ranked by protein abundance and taking the common hits to visualize in a log scale, with descending order based on average abundance of the final time point of individual samples.

DNA Next Generation Sequencing. Genomic DNA was purified by Qiagen Puregene kit (Qiagen). All strains that were sequenced are summarized in Table 2. Samples that are collected throughout the adaptive evolution were sequenced by either Illumina Miseq or Illumina Hiseq Rapid (Illumina), with a 2 × 150 bp pair-end format. Samples that were in the middle of adaptive evolution were all ensured to have a coverage of at least 60 to differentiate sequencing error from SNPs. Data was then processed by Geneious 11 software (Geneious), by trimming with BBduk and then mapped to a reference by the software’s native mapper. SNP variants were called by setting the criteria to a frequency of 25%.

The final stain was then sequenced by Pacbio Sequel (Pacific Biosciences) and Nanopore sequencing (Oxford Nanopore Technologies). For Pacbio Sequel, a 25 kb SMRTbell library (Pacific Biosciences) was prepared and its quality was assessed by fragment analyzer (Agilent). The library was then run in a CLR 20-hr diffusion mode. The reads were assembled by HGAP4. Nanopore sample preparation and sequencing were conducted by Health GeneTech Corp., Taiwan.

Digital PCR. CNVs were detected by droplet digital PCR (ddPCR) with standard protocols using the QX200TM ddPCR system (BioRad). Genomic DNA was first extracted as done in the NGS experiment. 0.5 µg of DNA was then digested with HindIII for 1 hr. The PCR reaction was carried out by a “ddPCR Supermix for Probes” kit (Biorad) after loading 25 pg of the digested DNA. Data was analyzed by QuantaSoft Analysis Pro Software.

Copy number variation Dynamics. SM1 was streaked out on a LB plate, where a single colony was picked and inoculated in LB. The LB culture was then passed to LB culture 3 more times, followed by a MM culture inoculation. The MM culture was then streaked out onto LB plates twice, where 7 colonies were then inoculated into LB once again. Data shown here starts from this point. The strain was then passed into LB for 7 times. The 1st, 4th and 7th pass (annotated as “Pas1”, “Pas4”, “Pas7” culture were passed into MM culture to test methanol growth. The genomic DNA of the LB culture and the subsequent MM culture were extracted by the Qiagen Puregene kit, where the copy number were tested by digital PCR.

qRT-PCR analysis. E. coli total RNA was prepared using RNeasy mini kit (Qiagen) and reverse transcribed by QuantiNova reverse transcription kit (Qiagen). Detection of cDNA levels were performed using CFX Connect™ Real-Time PCR detection system (BioRad Laboratories). All samples were measured in triplicate in hard-shell 96-well PCR plate using QuantiNova SYBR green RT-PCR kit (Qiagen). The expression fold change was analyzed by ΔΔCt values normalized to E. coli 16S rRNA. The overexpressed heterologous genes were categorized in formaldehyde consuming and producing gene data set. The data sets were first tested by a Shapiro-Wilk test to test the data is normally distributed, which was the case. A two-tailed F-test was the done to evaluate whether a t-test with equal of unequal variance should be used. Finally, the t-test was done to evaluate if the fold changes of formaldehyde consuming and producing genes are statistically different from each other.

RNA-seq analysis. E. coli total RNA was extracted by RNeasy mini kit (Qiagen). rRNA was prepped by Ribo-Zero (Bacteria) kit. Data was then processed by CLC genomics workbench 20. The TPM were calculated, and the following metabolic pathway includes the following gene when calculating the TPM distrubtion: TCA includes aspA, fdrA, fdrB, fumA, fumB, fumC, gltA, icd, mdh, mqo, ppc, prpC, prpD, sdhA, sdhB, sdhC, sdhD, sucA, sucB, sucC, sucD, yahF and ybhJ; EMP (Glycolysis) includes: aceE, aceF, cra, eno, fbaA, fbaB, gpmA, gpmM, lpd, pfkB, pgj, pykA, pykF, tpiA and gapC; ED includes: pgi, zwf, pgl, edd and eda; RuMP includes: medh, hps, phi, tal, tkt, rpe, and rpiA. The TPM sets sorted by metabolic pathway were evaluated if they are statistically different from others by the methodology mentioned in the qRT-PCR section.

Reverting deleted genes and assessment of their phenotypic effects. Native operons of pfkA, frmA, gapA were cloned into BAC (bacteria artificial chromosome) with an AmpR selection marker and transformed back to SM1 strain. SM1 strains re-expressing pfkA, frmA, gapA or both pfkA and gapA were then re-inoculated into 400 mM methanol medium. Growth curves were recorded and compared with SM1 strain transformed with an empty BAC.

Methanol consumption and fermentation product analysis. Samples were prepared by aliquoting the supernatant of the culture after centrifugation at 15000 rpm for 3 minutes and then filtered through a 0.22 filter (Milipore). Methanol concentration was determined by an Agilent Technologies 7890 gas chromatography with a flame-ionization-detector. Nitrogen gas with constant pressure of 19.082 psi was flowed through a DB-624UI column (Agilent Technologies, 0.32 mm × 30 m × 0.25 µm) a thermal cycle consisting of the following stages: initial 45° C. for 1 minute, ramp rate of 20° C./min to 150° C., and 45° C./min to 240° C. with a final 1-minute hold.

The fermentation products, namely acetate and formate, were measured by an Agilent 1290 UPLC using an Hi-plex H column (Agilent Technologies, 300×6.5 mm). A run was done with the mobile phase consisting 30 mM sulfuric acid with a flow rate of 0.6 mL/min for 30 minutes.

Quantification and Statistical Analysis. Details of statistical analysis can be found in the figure legends or in the method. All data are presented as means with error bars that indicates standard deviations, unless specified otherwise. Calculations are computed by Microsoft Excel, R, CLC Genomics Workbench 20, Geneious 2020 and Matlab 2019b.

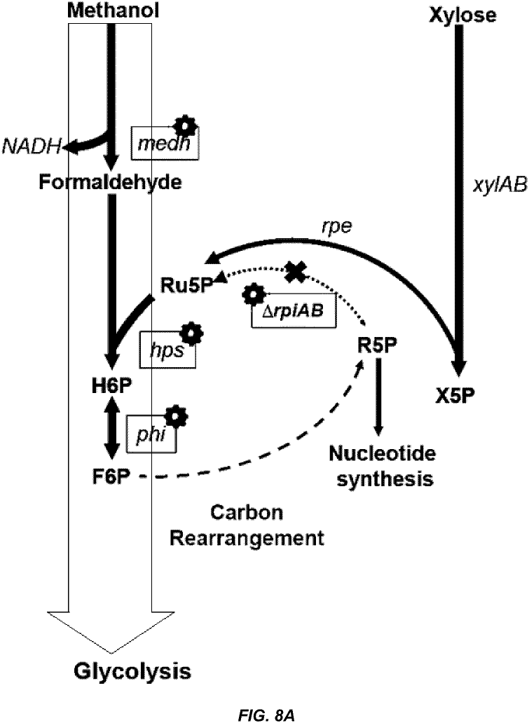

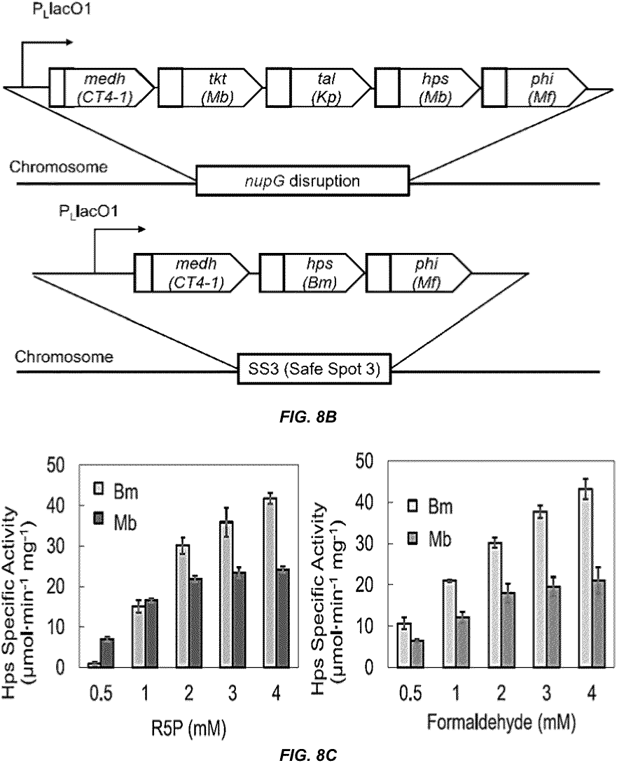

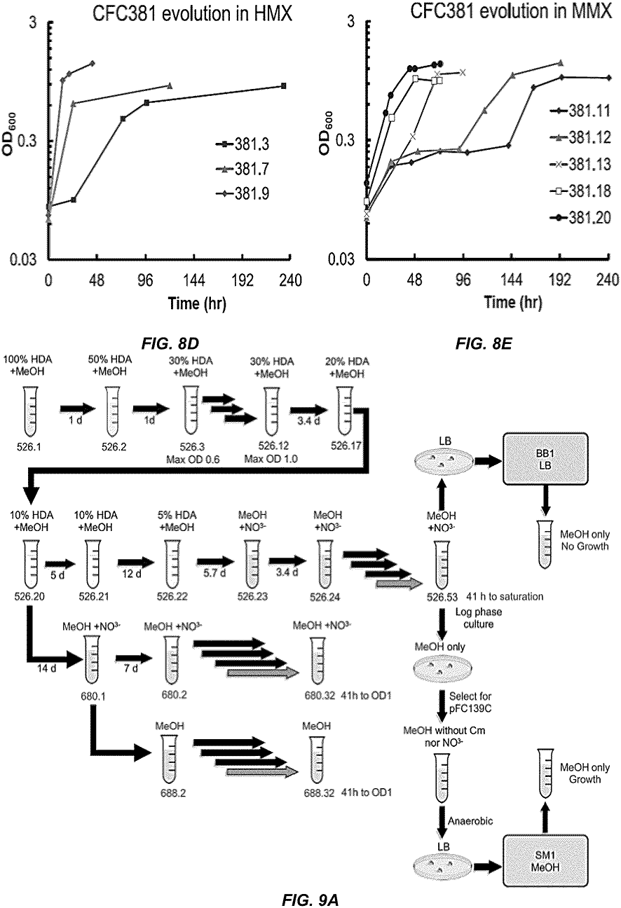

Methanol auxotrophy as a starting point. To develop a synthetic methylotroph, a RuMP cycle-based methanol auxotrophy strategy was used (FIG. 1B and FIG. 8A). It calls for a disruption of the pentose phosphate pathway by deleting the rpiAB gene and installing the methanol utilizing genes (medh, hps, phi), such that the cell can grow on methanol plus xylose in minimal media, but not on xylose alone. Thus, methanol assimilation can be used as a selection pressure during evolution. Instead of the previously established BL21 strain, a strategy was used to reconstruct an auxotrophic E. coli BW25113 ΔrpiAB strain mainly due to higher success rate of genome manipulation. Accordingly, two synthetic operons were integrated (FIG. 8B) for stable expression, designated as CFC381.0. The first operon consists of the three heterologous genes, medh (CT4-1, engineered from Cupriavidus necator), hps (from Bacillus methanolicus) and phi (from Methylobacillus flagellatus). The second operon includes the same medh and phi, but different hps (from Methylomicrobium buryatense 5GB1S) (FIG. 8C) along with tkt (encoding transketolase from Methylococcus capsulatus) and tal (encoding transaldolase from Klebsiella pneumoniae). As enzymes from different organisms differ in Kₘ or optimal substrate concentrations, various isofunctional enzymes were expressed simultaneously to maximize the flexibility of metabolic flux balance. After 20 liquid transfer cycles or “passages”, the evolved strain CFC381.20, could grow from OD₆₀₀ 0.1 to OD₆₀₀ 1.0 in a minimal medium containing 400 mM of methanol and 20 mM xylose in 48 hours (FIGS. 8D and 8E), but could not grow without methanol. Hence, CFC381.20 demonstrated the methanol auxotroph phenotype as desired.

Whole genome sequencing of CFC381.20 (Table 2) revealed a 4bp-insertion in the frmA gene. This suggests that the formaldehyde flux must be directed to biosynthetic pathways for efficient methanol-dependent growth. Other significant mutations included truncation of gnd (encoding 6-phosphogluconate dehydrogenase, Gnd) and a frameshift in fdoG (encoding formate dehydrogenase. Gnd forms a non-productive cycle with Hps, Phi, Pgi and Zwf, with a net reaction to convert formaldehyde to CO₂ and NADPH. Similarly, frmA and fdoG drains formaldehyde to CO₂ while producing excess NADH. These mutations indicated that through evolution the methanol auxotrophic strain reduced competing flux away from the productive RuMP cycle for efficient biosynthesis and biomass accumulation. This methanol auxotroph strain demonstrated that the methanol assimilation branch of the RuMP cycle was functional. However, the replenishment of ribulose-5-phosphate (Ru5P) was still supplied by xylose because the regeneration pathway was disrupted by the rpiAB deletion.

Rational design and evolution for creating a synthetic methylotroph. Next, experiments were perfomed to close the RuMP cycle by transforming a plasmid (pFC139) that carries an RBS library expressing rpiA, so that CFC381.20 could utilize methanol as the sole carbon source. Unfortunately, the strain could only acquire limited growth advantage after series of evolution in the presence of methanol while supplying limited nutrients, such as amino acids or xylose. It was hypothesized that kinetic traps in the RuMP cycle curtailed the flux during methanol assimilation. To identify them, Ensemble Modeling for Robustness Analysis (EMRA) (Lee et al., 2014; Rivera et al., 2015) was used, which examines a large number of models with different kinetic parameters, and perturbs them by varying enzyme Vₘₐₓ, which is largely proportional to expression levels. It then detects the models that become unstable after perturbation and reports the percentage of stable models after increasing or decreasing Vₘₐₓ. If a certain enzyme become unstable sharply after a small perturbation, then it may be involved in a kinetic trap. This analysis provides a qualitative way to suggest enzymes that require up or down regulation in order to facilitate the desirable metabolic flux distribution in the system.

Results revealed that high activities of phosphofructokinase (Pfk) and glyceraldehyde 3-phosphate dehydrogenase (Gapdh) tend to destabilize the system by diverting the flux away from the RuMP cycle (FIG. 2) and preventing the replenishment of the cycle intermediates. Accordingly, these enzyme activities were reduced by knocking out pfkA, which accounts for 90% of the Pfk activity, and replacing the gapA gene with the gapC gene from E. coli BL21 that possesses about 40% of K12 BW25113 GapA activity. The resulting strain, CFC526.0, was then subject to laboratory evolution with different strategies of nutrient weaning (FIG. 1B).

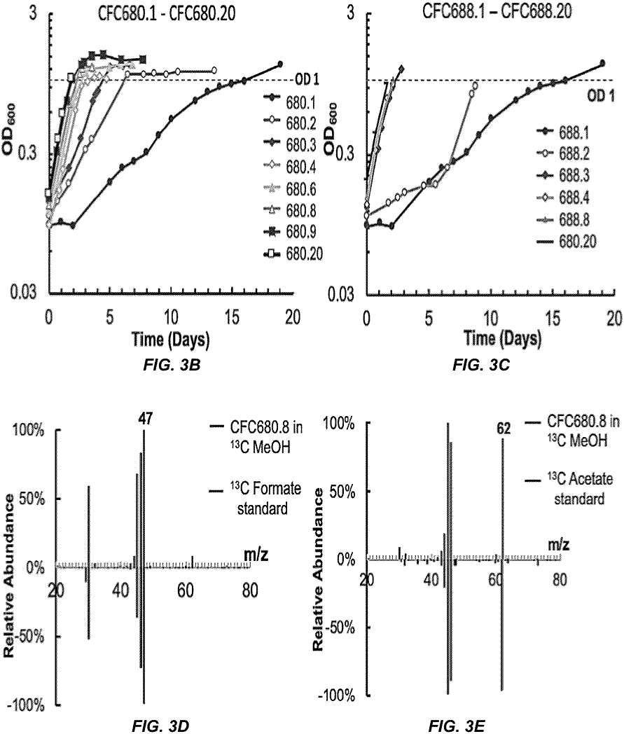

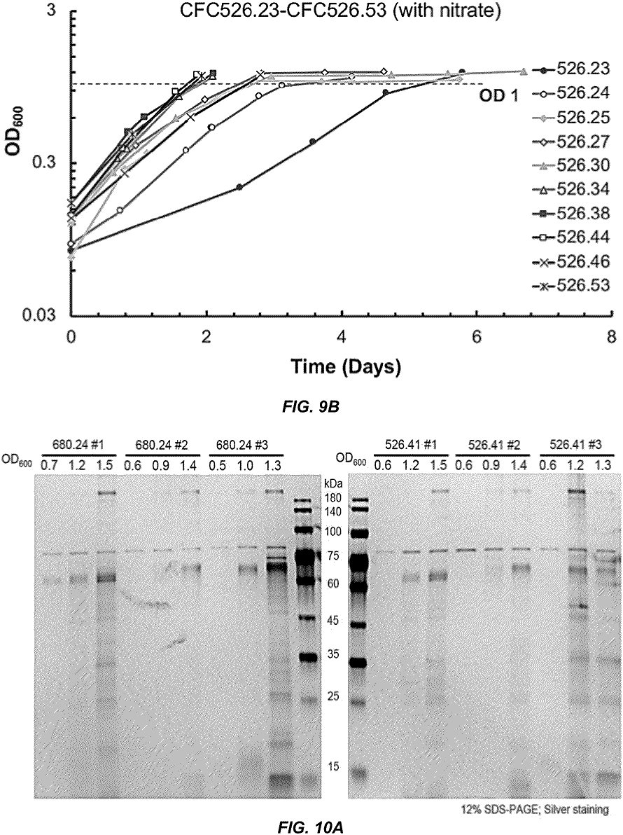

Specifically, CFC526.0 was grown in a medium containing methanol and a defined semi-minimal medium, Hi-Def azure (HDA) that contained amino acids. The HDA amount was sequentially reduced and replaced by the methanol MOPS (MM) minimal medium until the culture could grow on methanol as the sole carbon source. Extra vitamins were provided for better cell metabolism. Nitrate was also supplied as an extra electron acceptor in addition to oxygen, since methanol is an electron-rich substrate and oxygen transfer may be limiting in shaking-flasks. After about 180 days and 21 iterations, the culture could finally grow on methanol without any amino acid supplement (FIG. 3A and FIG. 9A). This initial methylotrophic culture CFC680.1 that grew solely on methanol required 20 days to grow to saturation at OD₆₀₀ = 1. After 20 more passages, CFC680.20 grew to OD₆₀₀ = 1 within 41 hours (FIG. 3B). The culture was evolved without supplying nitrate as well and generated a culture CFC688.20 that could grow without nitrate to reach a similar growth rate (FIG. 3C). Independently, another methanol-growing strain CFC526.23 was obtained by employing a slower nutrient reduction strategy and evolved it to obtain CFC526.53 (FIG. 9B).

To ensure that all metabolic products were derived from methanol, ¹³C labeling experiments were performed. CFC680.8 was passed six times in MM with ¹³C methanol until all isotopes reach a steady state. As expected, acetate was double-labeled, while formate was single-labeled (FIGS. 3D and 3E). Despite frmA being truncated, formate was still detected, which presumably was produced by either tetrahydrofolate-mediated metabolism or other unknown pathways. The isotope labeling experiments provided solid evidence that methanol was the sole carbon source for growth.

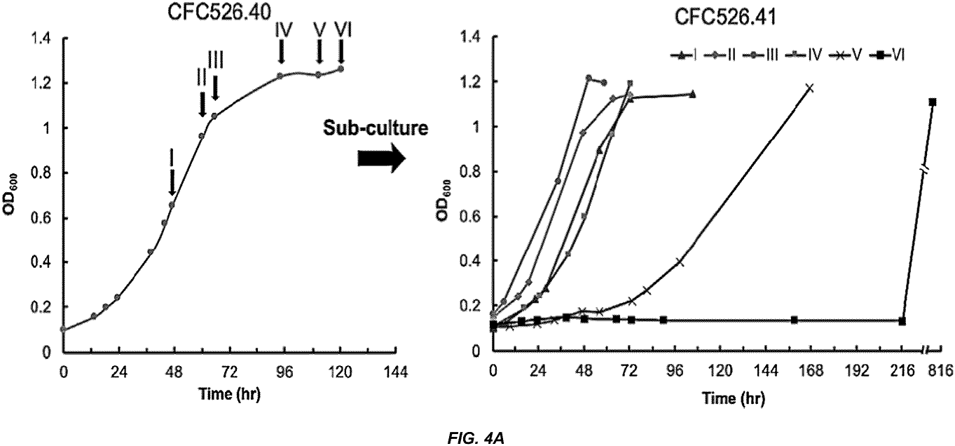

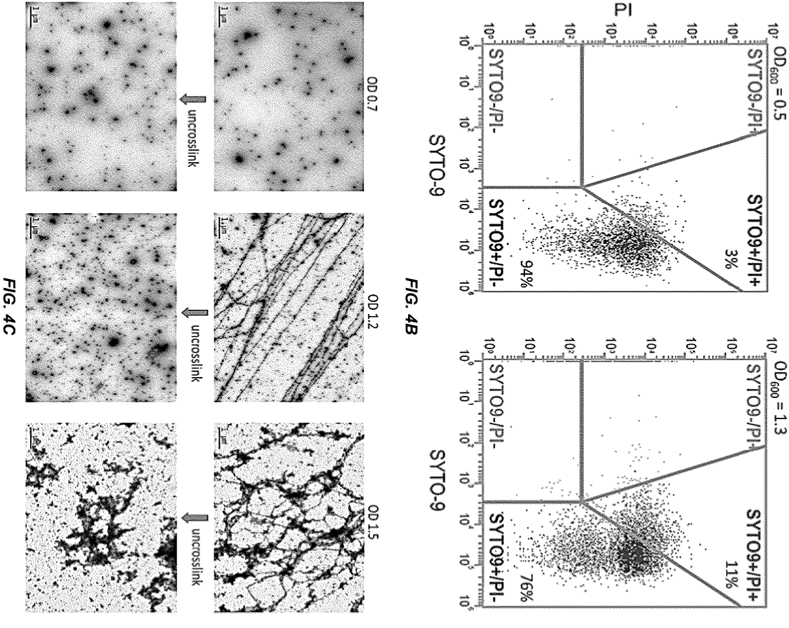

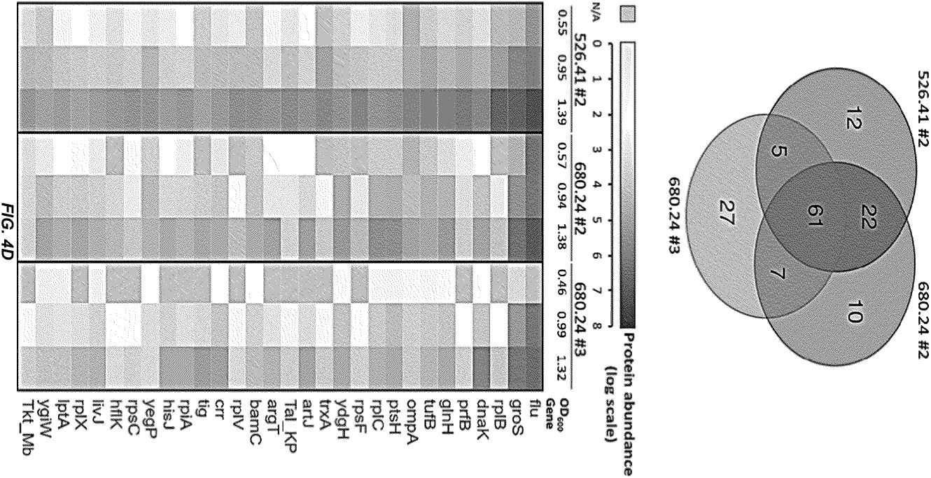

The DNA-protein crosslinking problem. One noticeable phenotype of these methylotrophic cultures was an exceedingly long lag phase (up to 20 days) if the culture was inoculated from the stationary phase (FIG. 4A), but not from the log-phase. Similarly, colonies on a methanol minimal medium plate could not proliferate in a liquid minimal medium. Although microorganisms do exhibit a lag phase when inoculated from a stationary-phase culture, these synthetic methylotrophic E. coli cultures seemed to go through a “point-of-no-return,” beyond which the exceedingly long lag phase appeared. After monitoring the viability of the cells at stationary phase by flow cytometry, the data showed that up to 10% of the cells were dead (FIG. 4B). The dead cells were stained with propidium iodide, indicating that the integrity of the cell membrane was damaged. Moreover, 7% cells had a significant shape distortion, according to the gated area of cell sorting. It was speculated that the strain may have experienced toxicity from intermediate metabolites, mostly likely due to formaldehyde accumulation. This was foreseeable as the inactivation of frmA inherited from the auxotrophy strain hindered the entire formaldehyde detoxification pathway.

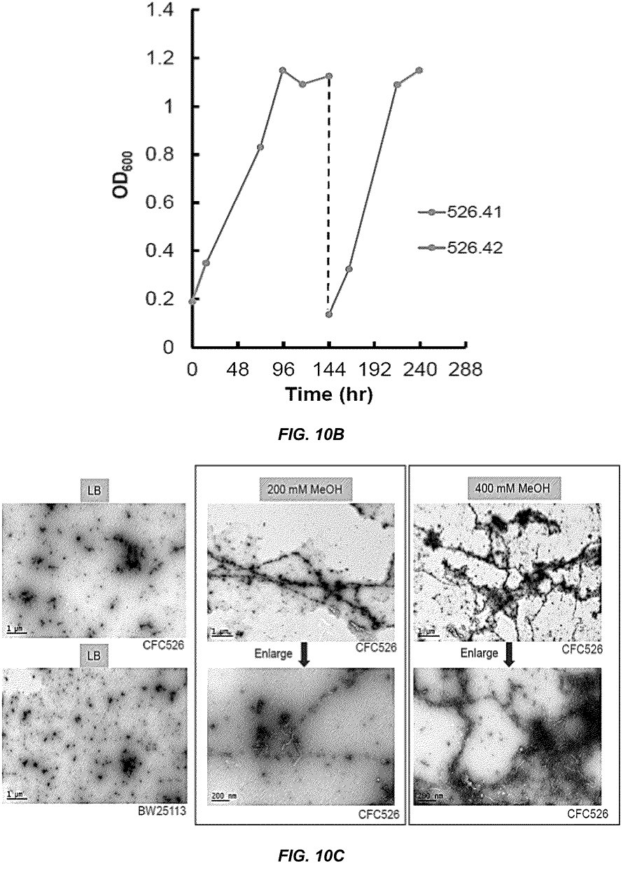

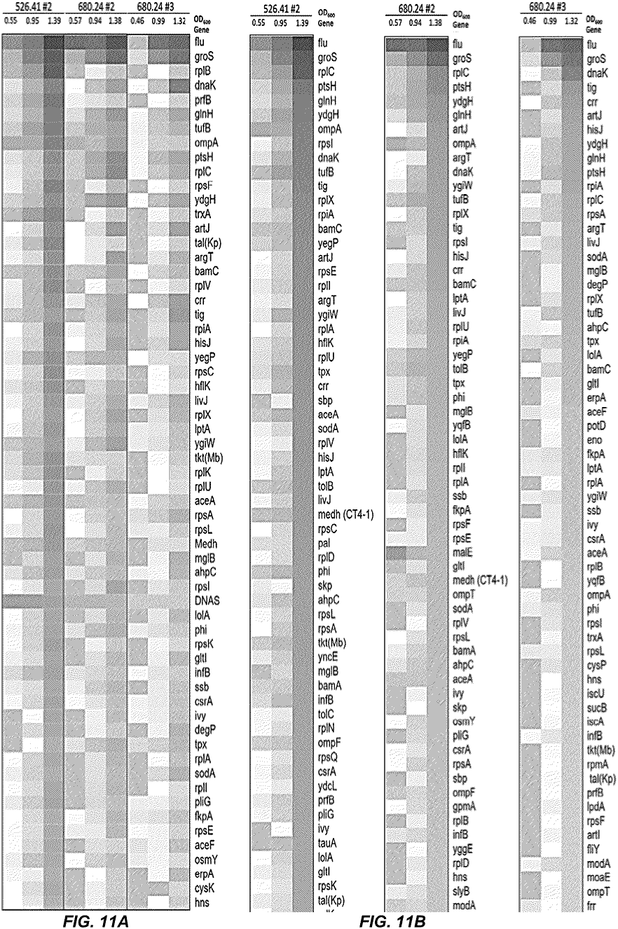

Surveying the broad spectrum of biomolecules susceptible to formaldehyde reactions, it was hypothesized that DNA-protein crosslinking (DPC) was the most likely cause of cell death which may lead to the disruption of DNA replication, transcription, translation and protein function (Stingele and Jentsch, 2015). To test the hypothesis, DPC products were purified from methanol-growing cultures by a modified DNA extraction method (Qiu and Wang, 2009b). After de-crosslinking the extractant, the protein portion was analyzed by SDS-PAGE. Results suggested that DPC did occur as the culture reached stationary phase (FIG. 10A). The isolated DPC products were then imaged using transmission electron microscope (TEM) and unveiled the severity of formaldehyde crosslinking (FIG. 4C). Typically, DNA could not be seen with negative staining without coating by proteins such as cytochrome C, as the DNA string is too thin for TEM observation.

As expected, during the log phase, only free protein particles were observed, most likely attributable to protein leftovers during the salting-out process. In contrast, the DPC level increased when the culture reached the stationary phase (O_{D600} 1.2), causing the entire DNA string to be visible as protein was coated to DNA due to formaldehyde crosslinking. Moreover, protein aggregates could be observed along the DNA string. At OD₆₀₀ 1.5, formaldehydeinduced crosslinking became extremely severe, where the DNA started to form a web-like structure by DNA-Protein-DNA crosslinking or even DNA-DNA crosslinking. Noticeably, DNA strings disappeared when heated and de-crosslinked DPCs, ruling out the possibility of DNA-protein nonspecific binding or image overlapping. DPC was less severe when the cells were growing in lower methanol concentrations (FIGS. 10B and 10C).

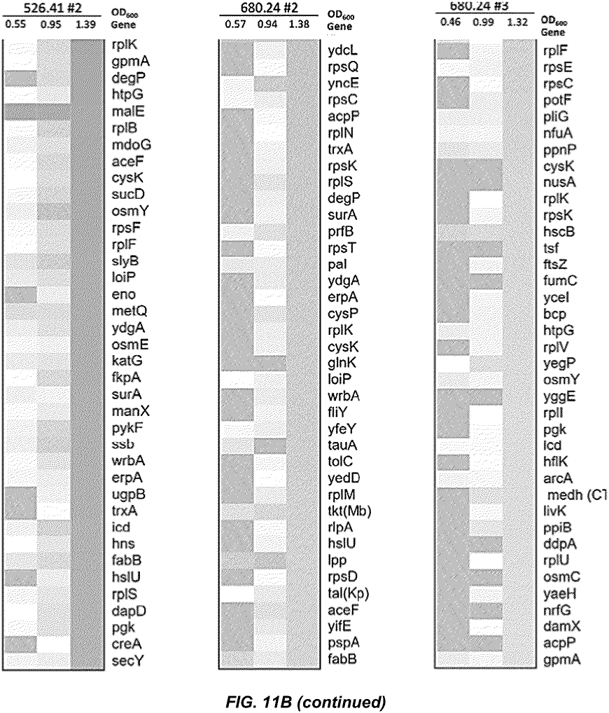

Quantitative proteomics was then conducted to reveal that more than 500 proteins were crosslinked with DNA. The common 61 hits of the 100 proteins with the highest abundance from 3 independent samples were then visualized with a heat map (FIG. 4D and FIG. 11). There was a trend of increasing crosslinked proteins as the culture enters the stationary phase, and the protein abundance in DPC products in the same culture could differ up to 7 orders of magnitude between the log phase and late stationary phase. Moreover, gene ontology analysis of the 61 proteins suggested that DPCs mainly consisted of ribosomes and outer membrane proteins, while several metabolic enzymes were also identified, such as Medh, Tkt, Tal, AceA, Eno, Pyk. Malfunction of these proteins may cause cell death due to outer membrane porin induced programmed cell death, or metabolic flux imbalance. Moreover, the strong presence of ribosomes also suggested that transcription and translation were heavily impacted by DPC as well. The accumulation of DPC could explain why the culture exhibits an exceedingly long lag phase when inoculated from a stationary phase culture, and may also shed light on the difficulty of evolving a non-methanol-utilizing bacterium to grow on methanol as the sole carbon source.

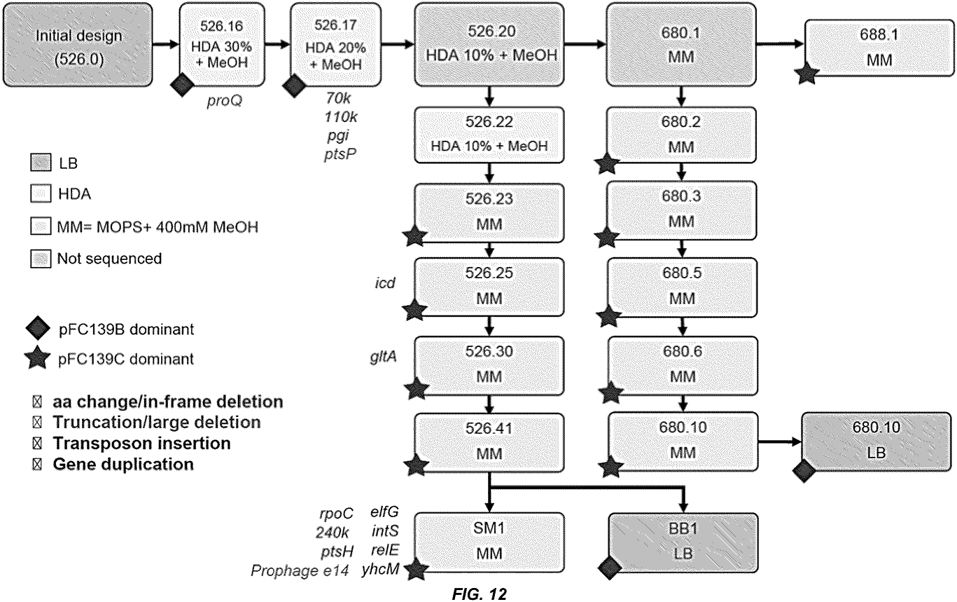

Genome sequencing revealing sub-populations in evolved cultures. Another phenomenon identified was that the cultures evolved to grow on methanol as the sole carbon source initially failed to grow in the same medium after passing through Luria-Bertani (LB) rich medium. This observation implied that the sub-populations emerged during evolution and were enriched in different media. To determine how CFC526.0 evolved to grow in methanol, the evolved cultures were sequenced along the evolution process (FIG. 5A and FIG. 12). Results showed that some mutations appeared but then vanished within a few passages. Along the evolution line, insertion sequence element 2 (IS2) was inserted upstream of two genes, gltA and ptsH that distances their promoter away from the open reading frame. Accordingly, the TCA-cycle activity may be impeded, while the ptsH encoded Hpr protein may be insufficiently expressed, causing a disruption in the pts system. Other mutations included a 12-bp in-frame deletion in pgi and truncated ptsP and proQ. Interestingly, the contents of the two operons that were integrated in nupG and SS3 site, which includes medh, hps, phi, tkt and tal, remained unchanged.

The evolved mixed cultures had three high coverage regions flanked by IS elements in their chromosomes: a 70k region spanning from yggE to yghO (FIG. 5B) that contains many glycolytic genes and a synthetic operon PLlacO1:: medh-tkt-tal-hps-phi in the RuMP pathway, a 7k region encoding the dipeptide transporter operon (ddp) (FIG. 5C), and a 130k region from rrsA to rrlB containing several 16S RNAs. The high coverage implies that the cells may have increased expression of genes in those regions.

The plasmid sequence also showed three different versions (FIG. 5D): one (pFC139A) that contained a specific RBS from the library; one (pFC139B) that contained a triplicated untranslated region (UTR) upstream to rpiA, and an IS2 insertion between the p15A replication origin and the cat gene; yet another one (pFC139C) that contained the same RBS as pFC139A, and an additional inserted IS2 before the promoter of cat gene.

To evaluate the genome variation through evolution, it was discovered that the copy number of the 70k region gradually increased up to 5.6 copies (FIG. 6A). Meanwhile, plasmid pFC139A and pFC139B dominated at the early stage of evolution, but pFC139C eventually dominated at the end of the evolution (FIG. 6B). Interestingly, the copy number dip in CFC526.17 and CFC526.23 coincided with the increase of pFC139B abundance (FIGS. 6A and 6B). On the other hand, the 70k and 130k repeats, the single nucleotide variations (SNVs) mentioned previously, and pFC139C disappeared after the culture was inoculated from MM to LB. Instead, the 7k repeated region and pFC139B were selected when the culture was grown in LB.

The coherent increase in the multi-copy 70k region and pFC139C along with some SNPs implies that there are two main sub-populations in the evolved CFC526 and CFC680 culture series: one real synthetic methylotrophic strain (SM1) containing pFC139C and the 70k multicopy region (FIG. 5B), and the other non-methylotrophic strain (BB1) containing pFC139B and the 7k multicopy region but not the 70k repeated region (FIG. 5C).

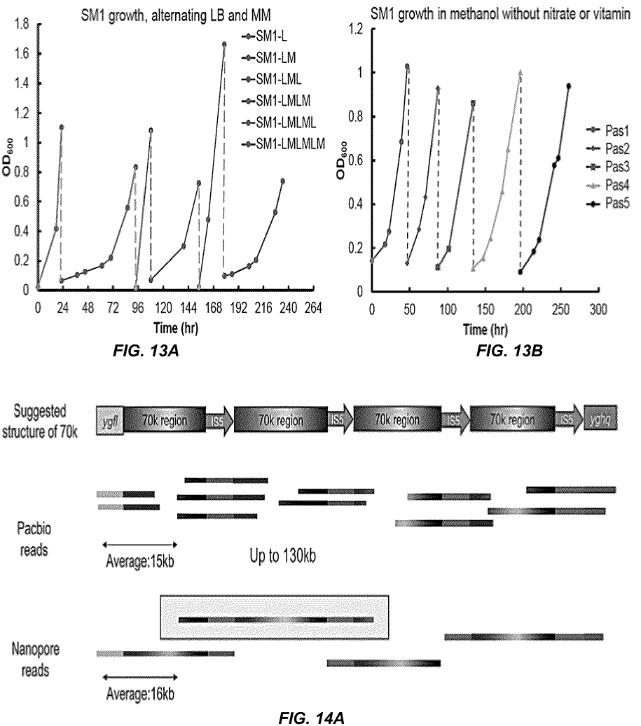

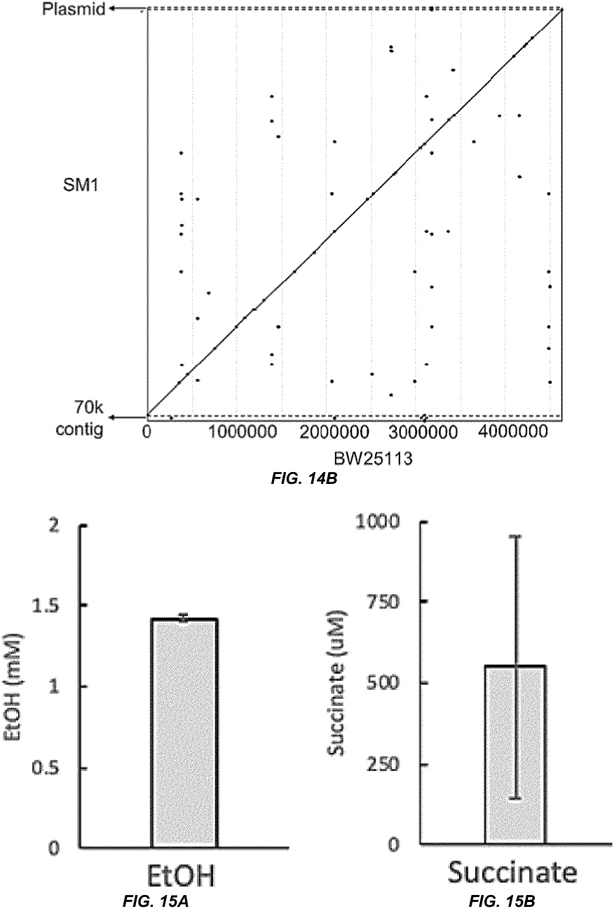

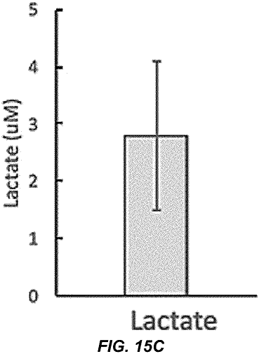

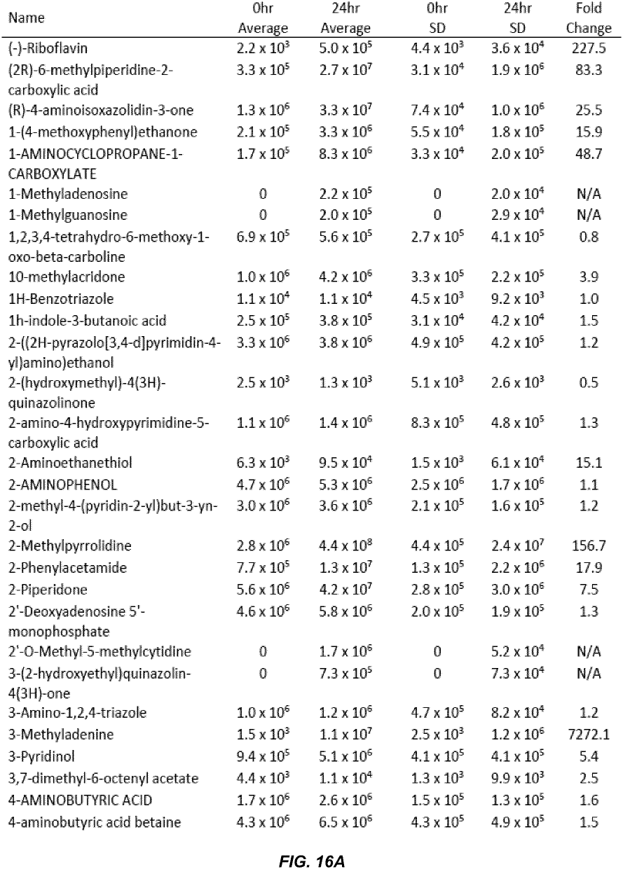

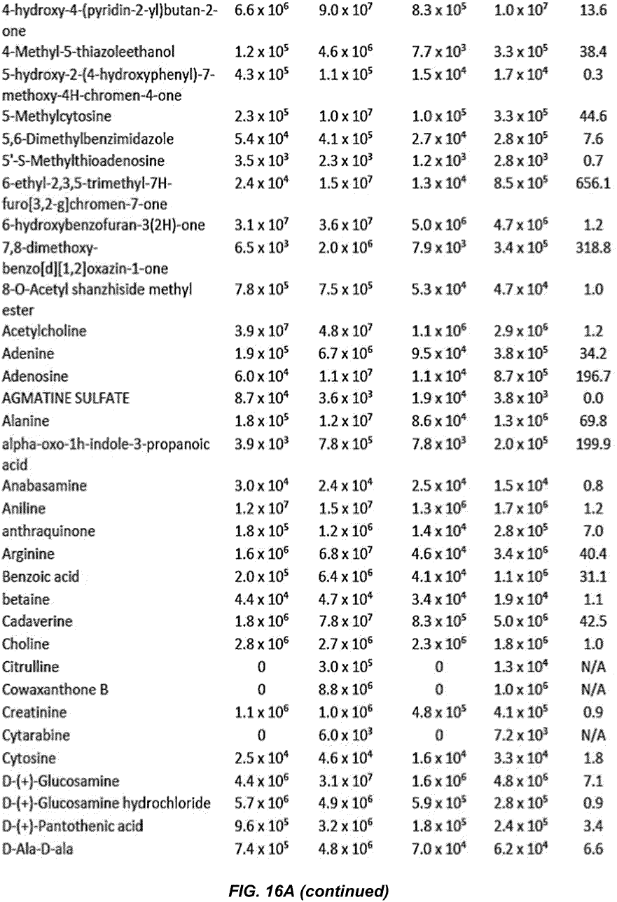

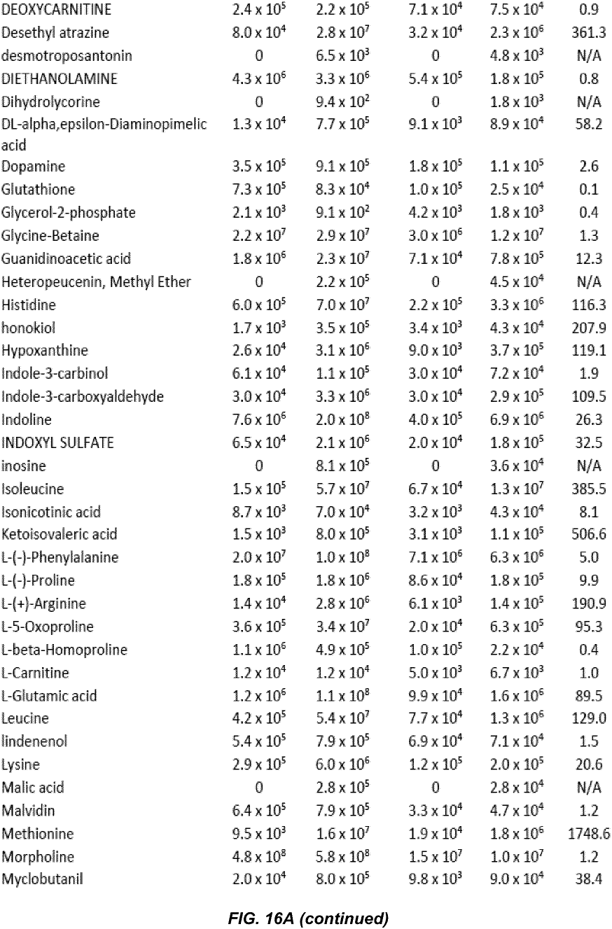

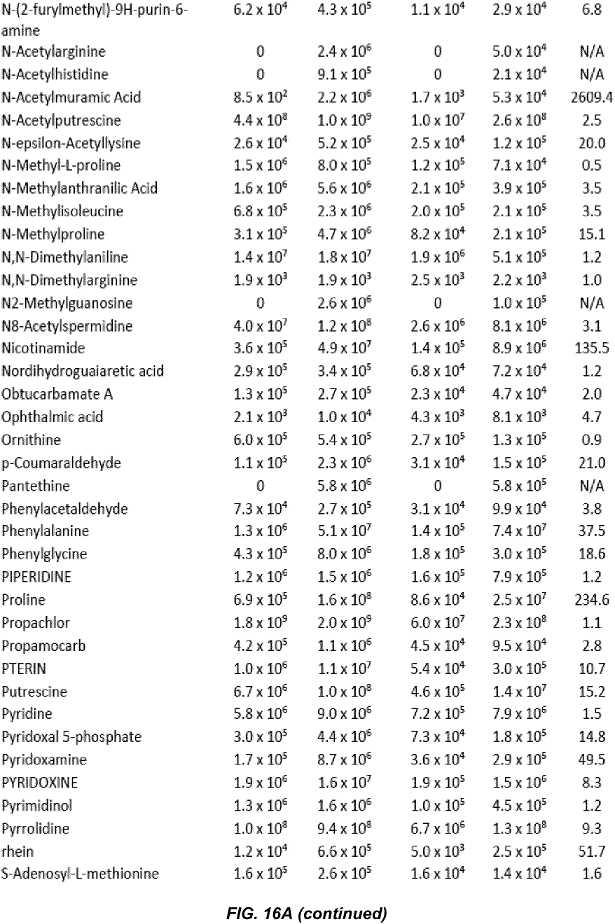

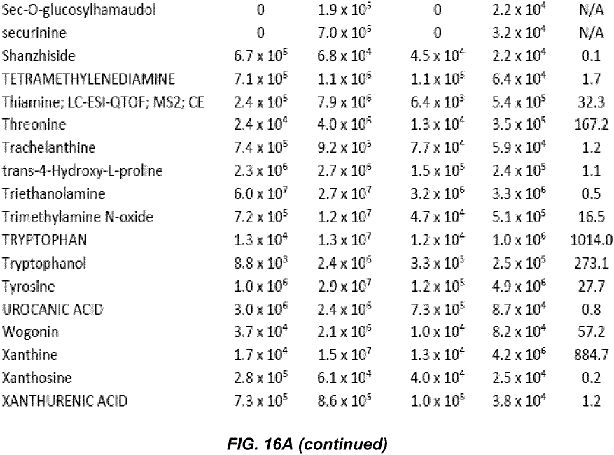

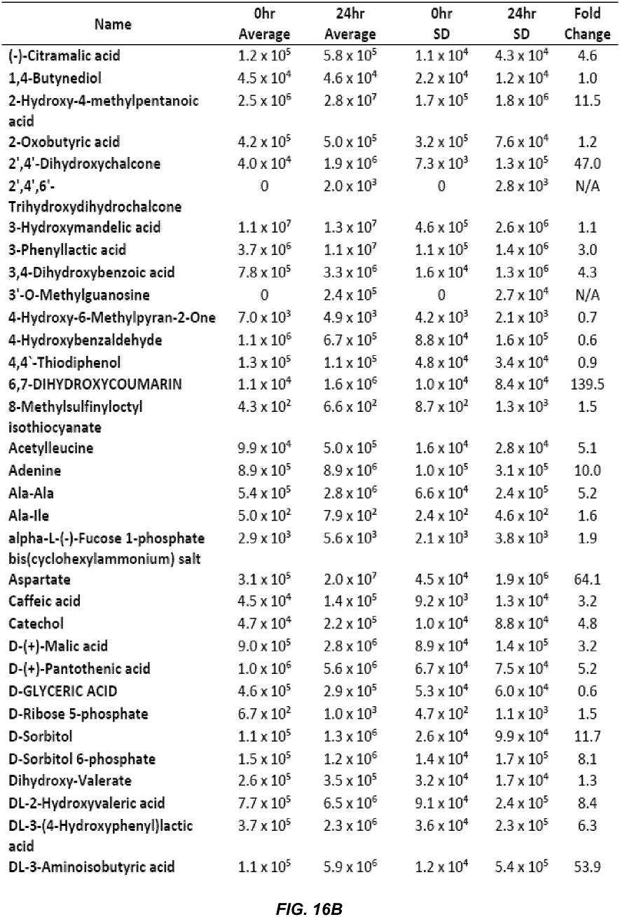

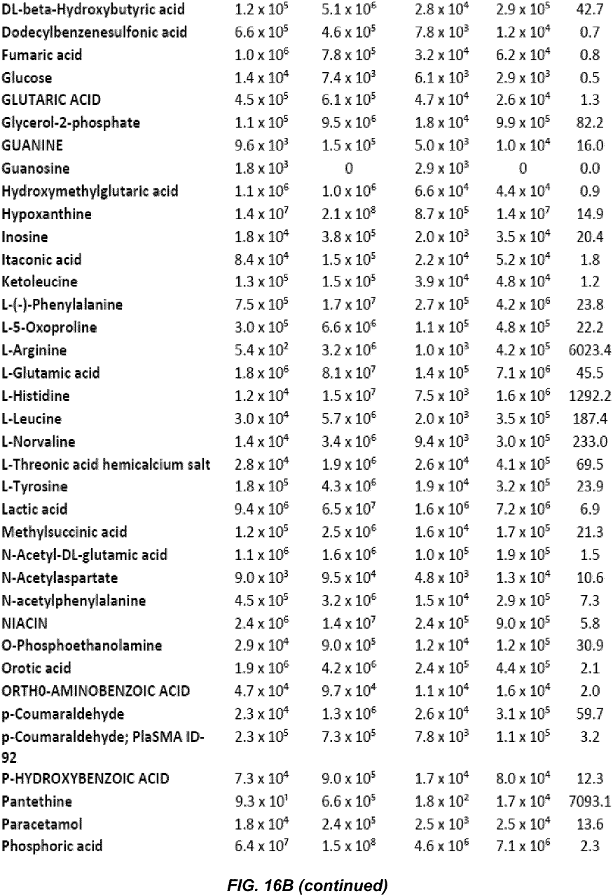

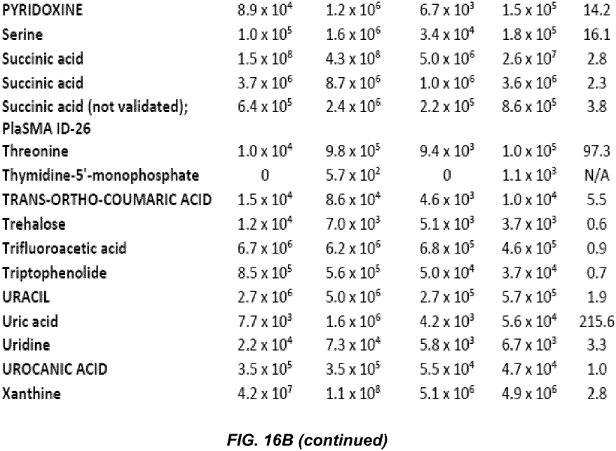

Isolation and characterization of a pure synthetic methylotrophic strain. After several attempts, SM1 and BB1 single colonies were isolated and identified by colony PCR verification of unique mutations such as pgi 12-bp deletion in SM1. As mentioned, before isolation of the SM1 strain, evolved cultures lost their ability to grow in methanol after passing in LB. This could be explained by the abrupt population shift from SM1 to BB1, where the latter could not grow in methanol at all when isolated. The final SM1 strain retains its ability to grow on methanol even after culturing in LB (FIG. 13A). Moreover, the strain can also grow without any nitrate or vitamin supplementation (FIG. 13B).

Illumina HiSeq sequencing of SM1 showed similar SNVs with increased frequency (close to 100%) compared to the last sequenced mixed culture, CFC526.30, except that some of the copy number variation (CNV) landscape changed (FIG. 6A). The 70k and 130k multicopy regions remained while another 240k duplicate appeared in SM1 (FIG. 5B). In contrast, the high coverage 7k region disappeared, which was later identified as a unique feature of BB1 strain (FIG. 5C).

To determine the genome structure, SM1 and BB1 were sequenced with Pacbio Sequel and Nanopore sequencing to seek longer reads. De novo assembly and mapping results from these long-read sequencing were instrumental for determining the genome structure and polishing the genome sequence. Several previously identified low-frequency SNVs from Hiseq sequencing were actually IS insertions (Table 2). These long sequencing reads (FIG. 14A) also revealed that the IS5-flanked 70k region consisted of tandem repeats (FIG. 5B). In particular, several ultra-long mapping reads (100~130 kb) from Nanopore sequencing that spanned three tandem repeats appeared (FIG. 14A). Comparing SM1 and the wild-type E. coli BW25113, several genomic structural variations were observed due to insertion sequences and CNVs (FIG. 14B).

Beneficial IS-mediated copy number variations. During evolution, the copy number of the 70k tandem repeat increased, leading to 4 copies in the isolated SM1 strain (FIG. 6A). The fine-tuning of CNV implies that the 70k-tandem repeats may play a role in synthetic methylotrophy as they host one of the artificially integrated operon, PLlacO1:: medh-tkt-tal-hps-phi, while also containing glycolysis and gluconeogenesis genes such as fbaA, pgk and yggF (a fructose-bisphosphatase isozyme) (FIG. 5B). The upregulation of the RuMP pathway enzymes may have enhanced the efficiency of methanol assimilation. The increase in yggF copy number may have further decreased Pfk flux, which is consistent with the EMRA prediction. The copy number of the 70k tandem repeat in SM1 was confirmed by digital PCR, Illumina sequencing, and long-read sequencing coverage data, which showed similar results. Noticeably, the copy number of the 70k reduced to 3 when the strain was grown in LB. On the other hand, the copy number of the 240k and 130k duplicated regions did not vary along the evolution path.

To further investigate the correlation between methanol growth and the 70k CNV, and the dynamics of CNV in SM1 strain, a single colony of SM1 strain was picked and passed it in LB 4 times, and then to methanol minimal medium (MM) to generate possible CNVs. Several isolated single colonies were then passed from the last MM culture through additional serial passages in LB, while tracking their 70k CNVs and their methanol growth abilities after LB exposure. The copy number of the 70k region decreased as the strain was more exposed to LB (FIG. 6C). Intriguingly, after passing the strain back to MM, the strain increased back its copy number (FIG. 6D). The copy number difference of the 70k region between cultures in LB and subsequent MM was not constant, as all cultures managed to recover back to a copy number more than 4.5. Also, their methanol growth ability was impacted as well, showing that there is a clear correlation between methanol growth rate and the 70k copy numbers (FIG. 6E). Moreover, the rate of copy number decrease across individual biological repeats at the same passage seems to be constant, suggesting that there might be a non-stochastic process underlying this phenomenon.

On the other hand, the 7k multi-copy region unique to the BB1 strain featured a remarkable 85-fold coverage (FIG. 5C). This region hosts the ddp operon that is a putative dipeptide transport and utilization, suggesting that BB1 may be co-evolved for the purpose of utilizing dipeptides derived from the debris of SM1 after cell death. After entering the stationary phase in the MM medium or passing through LB, this strain rapidly took over and dominated the culture. This explained the difficulty experienced in isolating SM1 from the evolved mixed culture when stains were isolated directly from LB plates.

Balancing the formaldehyde flux. Balancing the formaldehyde flux is useful to avoid DPC. This task is particularly challenging when the cell needs to replenish Ru5P to react with formaldehyde in methanol-only media. SM1 accomplished this task in the log phase but failed when it entered the stationary phase. An RNA-seq analysis of SM1 in the MM medium was performed and compared the mRNA transcript levels at OD₆₀₀ 1.1 to OD₆₀₀ 0.7. Indeed, the mRNA profile in the RuMP pathway was significantly altered in the stationary phase (FIG. 7A). The transcript levels of most RuMP genes responsible for the regeneration of Ru5P that reacts with formaldehyde were dramatically decreased, while the formaldehyde-forming gene (medh) was down-regulated less. Consequently, the flux imbalance caused the accumulation of formaldehyde. qRT-PCR methods were used to verify that the expression changes were consistent with the RNA-seq results (FIG. 7A). It appeared that the fine balance between formaldehyde-forming and formaldehyde consuming flux was crucial when the cells were going into the stationary phase with a very large change in their transcriptome (FIG. 7B). Note that the Entner-Doudoroff pathway (ED) was functional in the cell though its transcript is considerably lower than the EMP pathway. The ED pathway provides another route for entering the RuMP pathway to regenerate Ru5P, thus contributing formaldehyde consuming flux as well. Interestingly, the ED pathway genes were also down regulated more than the formaldehyde generation genes, medh, contributing to the DPC formation in the stationary phase.

Beneficial mutations for synthetic methylotrophy. An important reason for the success in evolving SM1 was the rational design guided by EMRA that involved the deletion of pfkA and gapA and expression of gapC. These genome changes were designed to direct more flux to replenish Ru5P to assimilate formaldehyde. To verify the importance of these genome edits along with other mutations introduced during laboratory evolution, certain changes were reversed in SM1 and their phenotypes tested. frmA, pfkA, gapA, pgi, gltA, ptsH, ptsP and proQ were cloned into a bacterial artificial chromosome (pBAC) under the bacterial native promoters. Results showed that reinstalling the wild type versions of these genes all caused a negative effect on methanol growth (FIG. 7C). Specifically, frmA, gapA, pgi and ptsP showed the most significant effects, indicating that these mutations were particularly beneficial to SM1 growth. Moreover, when both pfkA and gapA were simultaneously reintroduced, the strain almost stopped growing, requiring a 7-day recovery to grow back to OD₆₀₀ 1. Therefore, the rationally designed pfkA and gapA genome edits effectively created a path for genomic evolution towards efficient growth in methanol.

As mentioned previously, an IS2 insertion in the promoter region of gltA was identified. Re-expressing a copy of gltA on pBAC slightly reduced the growth rate, suggesting that the IS2 insertion played a role in SM1 growth. Moreover, RNA-seq data indicated that TCA cycle genes transcripts per million (TPM) were much lower than other major metabolic pathways such as glycolysis and RuMP cycle in SM1.

Pgi variant coded by the 12bp-deleted pgi gene in SM1 was expressed and His-tagged. Interestingly, this Pgi variant resulted in a higher specific activity, presumably increasing the flux through Zwf to produce NADPH for growth (FIG. 7D). NADPH in the wild type E. coli mainly comes from three sources: Icd in the TCA cycle, Gnd, and Zwf in the oxidative pentose phosphate pathway. Since Gnd is deleted and the TCA cycle activity is low as deduced from the RNA-seq data, Zwf may have become the major NADPH source for growth. In addition, the flux through Zwf directly enters the ED pathway generating G3P, which can be used to generate Ru5P for reuse in RuMP pathway to regenerate Ru5P for methylotrophic growth.

Growth characterization of SM1 strain. This strain could grow in a wide concentration range of methanol from 50 mM to 1.2 M as the sole carbon source, free of nitrate (FIG. 7E). Optimal growth was observed around 400 mM methanol, as the strain grew from OD₆₀₀ 0.1 to 1.0 in 30 hours with a doubling time of 8 hrs and consumed around 120 mM of methanol to reach a final OD₆₀₀ of 1.9. Formate and acetate were the major products (FIG. 7F).

A number of embodiments of the invention have been described. Nevertheless, it will be understood that various modifications may be made without departing from the spirit and scope of the invention. Accordingly, other embodiments are within the scope of the following claims.

What is claimed is:

1. A synthetic methylotroph (SM) that grows on methanol as a sole carbon source and has a doubling time (t_{D}) of less than 12 hours.

2. The SM of claim 1, wherein the SM expresses:
a polypeptide having methanol dehydrogenase activity,
a polypeptide having hexulose-6-phosphate synthase activity,
a polypeptide having 3-hexulose-6-phosphate isomerase activity and
comprises increased activity of a polypeptide having phosphoglucoisomerase activity,
wherein the SM can grow on methanol as the sole carbon source.

3. The SM of claim 1, wherein the SM contains deletions or reductions in expression or activity of one or more of a glyceraldehyde dehydrogenase polypeptide, phosphofructokinase polypeptide, S-(hydroxymethyl) glutathione dehydrogenase polypeptide, histidine-containing protein, and/or a ProQ polypeptide.

4. The SM of claim 1, wherein the SM has increased copy number variation of any region within the SM’s genome.

5. The SM of claim 1, wherein the SM has an increase of one or more copy number variations of 2 to 85 of a region between yggE to yghO, rrsA to rrIB, and/or ygiG to smf, and/or osmC to dosP.

6. The SM of claim 1, wherein the SM is obtained by engineering a parental microorganism selected from the group consisting of Escherichia, Bacillus, Clostridium, Enterobacter, Klebsiella, Enterobacteria, Mannheimia, Pseudomonas, Acinetobacter, Shewanella, Ralstonia, Geobacter, Zymomonas, Acetobacter, Geobacillus, Lactococcus, Streptococcus, Lactobacillus, Corynebacterium, Streptomyces, Propionibacterium, Synechocystis, Synechococcus, Cyanobacteria, Chlorobi, Deinococcus and Saccharomyces sp.

7. The SM of claim 6, wherein the parental microorganism is E. coli.

8. The SM of claim 1, wherein the SM further expresses a ribose-5-phosphate isomerase A.

9. The SM of claim 1, having the doubling time and product profile of ATCC deposit accession number PTA-126783, when grown on methanol.

10. A synthetic methylotroph designated Escherichia coli SM1 having ATCC accession no. PTA-126783.

11. A method for producing a metabolite, comprising growing a SM of claim 1 in a medium comprising methanol, wherein the methanol is the only carbon source for the SM microbe, whereby the metabolite is produced.

12. The method of claim 11, wherein the metabolite is selected from the group consisting of 4-carbon chemicals, diacids, 3-carbon chemicals, higher carboxylic acids, alcohols of higher carboxylic acids, carotenoids, cannabinoids, isoprenoids, and polyhydroxyalkanoates.

13. The method of claim 11, wherein the metabolite is selected from the group consisting of succinate, ethanol, and n-butanol.

14. A recombinant microorganism that grows on methanol and expresses:
a polypeptide having methanol dehydrogenase activity,
a polypeptide having hexulose-6-phosphate synthase activity,
a polypeptide having hexulose-6-phosphate isomerase activity and
comprises increased activity of a polypeptide having phosphoglucoisomerase activity.

15. A recombinant microorganism that assimilates a C1 carbon source and comprises a plurality of enzymes selected from the group consisting of Medh, Hps, Phi, Pgi, RpiA, Tkt, Tal and any combination thereof.

16. The recombinant microorganism of claim 15, wherein the microorganism is E. coli.

17. The recombinant microorganism of claim 15, further comprising a reduction or knockout of a gene selected from the group consisting of pfkA, gapA, frmA, ptsH, proQ and any combination thereof.

18. The recombinant microorganism of claim 15, further comprising an amplified region of the genome.

19. The SM of claim 1, wherein the recombinant microorganism expresses one or more heterologous polynucleotide or over-expression of one or more heterologous polynucleotide encoding a polypeptide having methanol dehydrogenase activity, hexulose-6-phosphate synthase activity, 6-phospho-3-hexulose isomerase activity, glucose phosphate isomerase activity and/or ribose-phosphate isomerase A activity, with a concomitant reduction or elimination of glyceraldehyde-3-phsophate dehydrogenase activity, reduction or elimination of S-(hydroxymethyl)glutathione dehydrogenase (FrmA) activity, reduction or deletion of phosphocarrier protein HPr (also referred to as Histidine-containing protein, HPr and/or PtsH) activity, and the reduction or elimination of ProQ provides, wherein the microorganism grows on methanol.


SEQUENCE LISTING (45 sequences)

SEQ ID NO: 1 - DNA, 633 bp, Bacillus subtilis
  CDS (1)..(633)
atggaattacagcttgcattagacctcgtcaacatcccagaagccattgagctcgtcaaagaggtagaacaatacatcgacgtagttgaaatcggaacaccggtcgtcattaatgaaggcctaagagccgttaaagaattaaaagaagcatttcctcaattgaaggttcttgcagacctgaaaatcatggatgccggaggctacgaaattatgaaagcgtcggaagcaggcgctgacatcatcaccgttttaggggctacagacgacgcaacgattaaaggcgcagtagaagaagccaaaaaacaaaagaagaaaatcttagtggacatgattaacgtgaaagatatcgagtcccgtgcgcaagaaattgacgcactcggtgttgactacatctgcgtccacactggctatgatcttcaagcagagggcaagaactctttcgaagaattaacgacaatcaaaaacaccgtaaaaaacgcaaaaaccgcaatcgcgggcggcatcaaacttgatacactgccagaagtgatcaagcaaaaccccgaccttgtcattgttgggggcggaattacaagcgcagctgataaggcagaaacagcttcaaaaatgaagcagctgattgtccaaggataa

SEQ ID NO: 2 - protein, 210 aa, Bacillus subtilis
MELQLALDLVNIPEAIELVKEVEQYIDVVEIGTPVVINEGLRAVKELKEAFPQLKVLADLKIMDAGGYEIMKASEAGADIITVLGATDDATIKGAVEEAKKQKKKILVDMINVKDIESRAQEIDALGVDYICVHTGYDLQAEGKNSFEELTTIKNTVKNAKTAIAGGIKLDTLPEVIKQNPDLVIVGGGITSAADKAETASKMKQLIVQG

SEQ ID NO: 3 - DNA, 687 bp, Methylobacillus flagellatum
  CDS (1)..(687)
gtggcaaaaccattagttcaaatggcattagattcactagatttcgatcagactgtagcgcttgctacgactgttgcaccacatgttgatattcttgaaatcggtactccttgtatcaagtacaacggtatcaagttgctggagactctccgcgcaaagttccctaacaacaagatcctggttgacctgaagaccatggatgctggtttttacgaagcagagcctttctacaaggcaggtgccgacatcgtgaccgtgctcggcactgctgacattggcacgatcaaaggcgtcattgatgttgccaacaaatacggcaagaaggctcaagtcgacctgatcaacgtgactgacaaggctgcacgcaccaaggaagtggccaagctcggcgctcacatcattggcgttcacactggtttggatcaacaggctgctggtcagacaccgtttgccgatctcaaccttgtttccagcctgaacctgggtgttgacatttccgtagctggtggcgtgaaggcgactaccgccaaacaagtggttgatgcaggtgccacaattgttgttgctggtgcggctatctatggtgctgccgatcctgctgctgctgctgctgaaatcagcgctgcggccaagggtacacaaagcagtggtggcctgtttggctggctgaagaaactgttcagctaa

SEQ ID NO: 4 - protein, 228 aa, Methylobacillus flagellatum
VAKPLVQMALDSLDFDQTVALATTVAPHVDILEIGTPCIKYNGIKLLETLRAKFPNNKILVDLKTMDAGFYEAEPFYKAGADIVTVLGTADIGTIKGVIDVANKYGKKAQVDLINVTDKAARTKEVAKLGAHIIGVHTGLDQQAAGQTPFADLNLVSSLNLGVDISVAGGVKATTAKQVVDAGATIVVAGAAIYGAADPAAAAAEISAAAKGTQSSGGLFGWLKKLFS

SEQ ID NO: 5 - DNA, 1152 bp, Bacillus methanolicus
  CDS (1)..(1152)
atgacgcaaagaaactttttcattccaccagctagcgtaattggacgcggcgctgtaaaagaagtaggaacaagacttaagcaaattggagctacaaaagcacttatcgttacagatgcatttcttcatggcacaggtttgtcagaagaagttgctaaaaacattcgtgaagctggccttgatgctgtaattttcccaaaagctcaaccagatccagcagatacacaagttcatgaaggcgtagatatattcaaacaagaaaaatgtgatgcacttgtttctatcggtggaggtagctctcacgatacagcaaaagcaatcggtttagttgcagcaaacggcggaagaatcaacgactatcaaggtgtaaacagtgtagaaaaaccggttgttccagtagttgcaatcactacaacagctggtactggtagtgaaacaacatctcttgcggttattacagattctgcacgtaaagtaaaaatgccagttatcgatgagaaaattacaccaactgtagcaattgttgacccagaattaatggtgaaaaaaccagctggattaacaattgcaactggtatggatgcattatcccatgcaattgaagcatatgttgcaaaacgtgctacaccagttactgatgcgtttgcaattcaagcaatgaaactcattaatgaatacttaccacgtgcggttgcaaatggagaagacatcgaagcacgtgaagcaatggcttatgcacaatacatggcaggagtggcatttaacaacggaggtttaggattagtacactctatttctcaccaagtaggtggagtttacaagttacaacacggaatctgtaactcagttaatatgccacacgtttgccaattcaacttaattgctcgtactgaacgcttcgcacacattgctgagcttttaggcgagaatgtttctggcttaagcactgcatctgctgctgagagagcaattgtagcgcttcaacgctataacaaaaacttcggtatcccatctggctatgcagaaatgggcgtaaaagaagaggatatcgaattattagcgaacaacgcgtaccaagacgtatgtactctagataacccacgtgttcctactgttcaagacattgcacaaatcatcaaaaacgctctgtaa

SEQ ID NO: 6 - protein, 383 aa, Bacillus methanolicus
MTQRNFFIPPASVIGRGAVKEVGTRLKQIGATKALIVTDAFLHGTGLSEEVAKNIREAGLDAVIFPKAQPDPADTQVHEGVDIFKQEKCDALVSIGGGSSHDTAKAIGLVAANGGRINDYQGVNSVEKPVVPVVAITTTAGTGSETTSLAVITDSARKVKMPVIDEKITPTVAIVDPELMVKKPAGLTIATGMDALSHAIEAYVAKRATPVTDAFAIQAMKLINEYLPRAVANGEDIEAREAMAYAQYMAGVAFNNGGLGLVHSISHQVGGVYKLQHGICNSVNMPHVCQFNLIARTERFAHIAELLGENVSGLSTASAAERAIVALQRYNKNFGIPSGYAEMGVKEEDIELLANNAYQDVCTLDNPRVPTVQDIAQIIKNAL

SEQ ID NO: 7 - DNA, 1650 bp, Escherichia coli
  CDS (1)..(1650)
atgaaaaacatcaatccaacgcagaccgctgcctggcaggcactacagaaacacttcgatgaaatgaaagacgttacgatcgccgatctttttgctaaagacggcgatcgtttttctaagttctccgcaaccttcgacgatcagatgctggtggattactccaaaaaccgcatcactgaagagacgctggcgaaattacaggatctggcgaaagagtgcgatctggcgggcgcgattaagtcgatgttctctggcgagaagatcaaccgcactgaaaaccgcgccgtgctgcacgtagcgctgcgtaaccgtagcaataccccgattttggttgatggcaaagacgtaatgccggaagtcaacgcggtgctggagaagatgaaaaccttctcagaagcgattatttccggtgagtggaaaggttataccggcaaagcaatcactgacgtagtgaacatcgggatcggcggttctgacctcggcccatacatggtgaccgaagctctgcgtccgtacaaaaaccacctgaacatgcactttgtttctaacgtcgatgggactcacatcgcggaagtgctgaaaaaagtaaacccggaaaccacgctgttcctggtagcatctaaaaccttcaccactcaggaaactatgaccaacgcccatagcgcgcgtgactggttcctgaaagcggcaggtgatgaaaaacacgttgcaaaacactttgcggcgctttccaccaatgccaaagccgttggcgagtttggtattgatactgccaacatgttcgagttctgggactgggttggcggccgttactctttgtggtcagcgattggcctgtcgattgttctctccatcggctttgataacttcgttgaactgctttccggcgcacacgcgatggacaagcatttctccaccacgcctgccgagaaaaacctgcctgtactgctggcgctgattggcatctggtacaacaatttctttggtgcggaaactgaagcgattctgccgtatgaccagtatatgcaccgtttcgcggcgtacttccagcagggcaatatggagtccaacggtaagtatgttgaccgtaacggtaacgttgtggattaccagactggcccgattatctggggtgaaccaggcactaacggtcagcacgcgttctaccagctgatccaccagggaaccaaaatggtaccgtgcgatttcatcgctccggctatcacccataacccgctctctgatcatcaccagaaactgctgtctaacttcttcgcccagaccgaagcgctggcgtttggtaaatcccgcgaagtggttgagcaggaatatcgtgatcagggtaaagatccggcaacgcttgactacgtggtgccgttcaaagtattcgaaggtaaccgcccgaccaactccatcctgctgcgtgaaatcactccgttcagcctgggtgcgttgattgcgctgtatgagcacaaaatctttactcagggcgtgatcctgaacatcttcaccttcgaccagtggggcgtggaactgggtaaacagctggcgaaccgtattctgccagagctgaaagatgataaagaaatcagcagccacgatagctcgaccaatggtctgattaaccgctataaagcgtggcgcggttaa

SEQ ID NO: 8 - protein, 549 aa, Escherichia coli
MKNINPTQTAAWQALQKHFDEMKDVTIADLFAKDGDRFSKFSATFDDQMLVDYSKNRITEETLAKLQDLAKECDLAGAIKSMFSGEKINRTENRAVLHVALRNRSNTPILVDGKDVMPEVNAVLEKMKTFSEAIISGEWKGYTGKAITDVVNIGIGGSDLGPYMVTEALRPYKNHLNMHFVSNVDGTHIAEVLKKVNPETTLFLVASKTFTTQETMTNAHSARDWFLKAAGDEKHVAKHFAALSTNAKAVGEFGIDTANMFEFWDWVGGRYSLWSAIGLSIVLSIGFDNFVELLSGAHAMDKHFSTTPAEKNLPVLLALIGIWYNNFFGAETEAILPYDQYMHRFAAYFQQGNMESNGKYVDRNGNVVDYQTGPIIWGEPGTNGQHAFYQLIHQGTKMVPCDFIAPAITHNPLSDHHQKLLSNFFAQTEALAFGKSREVVEQEYRDQGKDPATLDYVVPFKVFEGNRPTNSILLREITPFSLGALIALYEHKIFTQGVILNIFTFDQWGVELGKQLANRILPELKDDKEISSHDSSTNGLINRYKAWRG

SEQ ID NO: 9 - DNA, 1638 bp, Artificial Sequence
  Mutant PGi derived from E. coli
  CDS (1)..(1638)
atgaaaaacatcaatccaacgcagaccgctgcctggcaggcactacagaaacacttcgatgaaatgaaagacgttacgatcgccgatctttttgctaaagacggcgatcgtttttctaagttctccgcaaccttcgacgatcagatgctggtggattactccaaaaaccgcatcactgaagagacgctggcgaaattacaggatctggcgaaagagtgcgatctggcgggcgcgattaagtcgatgttctctggcgagaagatcaaccgcactgaaaaccgcgccgtgctgcacgtagcgctgcgtaaccgtagcaataccccgattttggttgatggcaaagacgtaatgccggaagtcaacgcggtgctggagaagatgaaaaccttctcagaagcgattatttccggtgagtggaaaggttataccggcaaagcaatcactgacgtagtgaacatcgggatcggcggttctgacctcggcccatacatggtgaccgaagctctgcgtccgtacaaaaaccacctgaacatgcactttgtttctaacgtcgatgggactcacatcgcggaagtgctgaaaaaagtaaacccggaaaccacgctgttcctggtagcatctaaaaccttcaccactcaggaaactatgaccaacgcccatagcgcgcgtgactggttcctgaaagcggcaggtgatgaaaaacactttgcggcgctttccaccaatgccaaagccgttggcgagtttggtattgatactgccaacatgttcgagttctgggactgggttggcggccgttactctttgtggtcagcgattggcctgtcgattgttctctccatcggctttgataacttcgttgaactgctttccggcgcacacgcgatggacaagcatttctccaccacgcctgccgagaaaaacctgcctgtactgctggcgctgattggcatctggtacaacaatttctttggtgcggaaactgaagcgattctgccgtatgaccagtatatgcaccgtttcgcggcgtacttccagcagggcaatatggagtccaacggtaagtatgttgaccgtaacggtaacgttgtggattaccagactggcccgattatctggggtgaaccaggcactaacggtcagcacgcgttctaccagctgatccaccagggaaccaaaatggtaccgtgcgatttcatcgctccggctatcacccataacccgctctctgatcatcaccagaaactgctgtctaacttcttcgcccagaccgaagcgctggcgtttggtaaatcccgcgaagtggttgagcaggaatatcgtgatcagggtaaagatccggcaacgcttgactacgtggtgccgttcaaagtattcgaaggtaaccgcccgaccaactccatcctgctgcgtgaaatcactccgttcagcctgggtgcgttgattgcgctgtatgagcacaaaatctttactcagggcgtgatcctgaacatcttcaccttcgaccagtggggcgtggaactgggtaaacagctggcgaaccgtattctgccagagctgaaagatgataaagaaatcagcagccacgatagctcgaccaatggtctgattaaccgctataaagcgtggcgcggttaa

SEQ ID NO: 10 - protein, 545 aa, Artificial Sequence
  Synthetic Construct
MKNINPTQTAAWQALQKHFDEMKDVTIADLFAKDGDRFSKFSATFDDQMLVDYSKNRITEETLAKLQDLAKECDLAGAIKSMFSGEKINRTENRAVLHVALRNRSNTPILVDGKDVMPEVNAVLEKMKTFSEAIISGEWKGYTGKAITDVVNIGIGGSDLGPYMVTEALRPYKNHLNMHFVSNVDGTHIAEVLKKVNPETTLFLVASKTFTTQETMTNAHSARDWFLKAAGDEKHFAALSTNAKAVGEFGIDTANMFEFWDWVGGRYSLWSAIGLSIVLSIGFDNFVELLSGAHAMDKHFSTTPAEKNLPVLLALIGIWYNNFFGAETEAILPYDQYMHRFAAYFQQGNMESNGKYVDRNGNVVDYQTGPIIWGEPGTNGQHAFYQLIHQGTKMVPCDFIAPAITHNPLSDHHQKLLSNFFAQTEALAFGKSREVVEQEYRDQGKDPATLDYVVPFKVFEGNRPTNSILLREITPFSLGALIALYEHKIFTQGVILNIFTFDQWGVELGKQLANRILPELKDDKEISSHDSSTNGLINRYKAWRG

SEQ ID NO: 11 - protein, 85 aa, Escherichia coli
MFQQEVTITAPNGLHTRPAAQFVKEAKGFTSEITVTSNGKSASAKSLFKLQTLGLTQGTVVTISAEGEDEQKAVEHLVKLMAELE

SEQ ID NO: 12 - protein, 232 aa, Escherichia coli
MENQPKLNSSKEVIAFLAERFPHCFSAEGEARPLKIGIFQDLVDRVAGEMNLSKTQLRSALRLYTSSWRYLYGVKPGATRVDLDGNPCGELDEQHVEHARKQLEEAKARVQAQRAEQQAKKREAAATAGEKEDAPRRERKPRPTTPRRKEGAERKPRAQKPVEKAPKTVKAPREEQHTPVSDISALTVGQALKVKAGQNAMDATVLEITKDGVRVQLNSGMSLIVRAEHLVF

SEQ ID NO: 13 - DNA, 660 bp, Escherichia coli
  CDS (1)..(660)
atgacgcaggatgaattgaaaaaagcagtaggatgggcggcacttcagtatgttcagcccggcaccattgttggtgtaggtacaggttccaccgccgcacactttattgacgcgctcggtacaatgaaaggccagattgaaggggccgtttccagttcagatgcttccactgaaaaactgaaaagcctcggcattcacgtttttgatctcaacgaagtcgacagccttggcatctacgttgatggcgcagatgaaatcaacggccacatgcaaatgatcaaaggcggcggcgcggcgctgacccgtgaaaaaatcattgcttcggttgcagaaaaatttatctgtattgcagacgcttccaagcaggttgatattctgggtaaattcccgctgccagtagaagttatcccgatggcacgtagtgcagtggcgcgtcagctggtgaaactgggcggtcgtccggaataccgtcagggcgtggtgaccgataatggcaacgtgatcctcgacgtccacggcatggaaatccttgacccgatagcgatggaaaacgccataaatgcgattcctggcgtggtgactgttggcttgtttgctaaccgtggcgcggacgttgcgctgattggcacacctgacggtgtcaaaaccattgtgaaatga

SEQ ID NO: 14 - protein, 219 aa, Escherichia coli
MTQDELKKAVGWAALQYVQPGTIVGVGTGSTAAHFIDALGTMKGQIEGAVSSSDASTEKLKSLGIHVFDLNEVDSLGIYVDGADEINGHMQMIKGGGAALTREKIIASVAEKFICIADASKQVDILGKFPLPVEVIPMARSAVARQLVKLGGRPEYRQGVVTDNGNVILDVHGMEILDPIAMENAINAIPGVVTVGLFANRGADVALIGTPDGVKTIVK

SEQ ID NO: 15 - protein, 333 aa, Escherichia coli
MSKVGINGFGRIGRLVLRRLLEVKSNIDVVAINDLTSPKILAYLLKHDSNYGPFPWSVDFTEDSLIVDGKSIAVYAEKEAKNIPWKAKGAEIIVECTGFYTSAEKSQAHLDAGAKKVLISAPAGEMKTIVYNVNDDTLDGNDTIVSVASCTTNCLAPMAKALHDSFGIEVGTMTTIHAYTGTQSLVDGPRGKDLRASRAAAENIIPHTTGAAKAIGLVIPELSGKLKGHAQRVPVKTGSVTELVSILGKKVTAEEVNNALKQATTNNESFGYTDEEIVSSDIIGSHFGSVFDATQTEITAVGDLQLVKTVAWYDNEYGFVTQLIRTLEKFAKL

SEQ ID NO: 16 - DNA, 954 bp, Escherichia coli
  CDS (1)..(954)
atgacggacaaattgacctcccttcgtcagtacaccaccgtagtggccgacactggggacatcgcggcaatgaagctgtatcaaccgcaggatgccacaaccaacccttctctcattcttaacgcagcgcagattccggaataccgtaagttgattgatgatgctgtcgcctgggcgaaacagcagagcaacgatcgcgcgcagcagatcgtggacgcgaccgacaaactggcagtaaatattggtctggaaatcctgaaactggttccgggccgtatctcaactgaagttgatgcgcgtctttcctatgacaccgaagcgtcaattgcgaaagcaaaacgcctgatcaaactctacaacgatgctggtattagcaacgatcgtattctgatcaaactggcttctacctggcagggtatccgtgctgcagaacagctggaaaaagaaggcatcaactgtaacctgaccctgctgttctccttcgctcaggctcgtgcttgtgcggaagcgggcgtgttcctgatctcgccgtttgttggccgtattcttgactggtacaaagcgaataccgataagaaagagtacgctccggcagaagatccgggcgtggtttctgtatctgaaatctaccagtactacaaagagcacggttatgaaaccgtggttatgggcgcaagcttccgtaacatcggcgaaattctggaactggcaggctgcgaccgtctgaccatcgcaccggcactgctgaaagagctggcggagagcgaaggggctatcgaacgtaaactgtcttacaccggcgaagtgaaagcgcgtccggcgcgtatcactgagtccgagttcctgtggcagcacaaccaggatccaatggcagtagataaactggcggaaggtatccgtaagtttgctattgaccaggaaaaactggaaaaaatgatcggcgatctgctgtaa

SEQ ID NO: 17 - protein, 317 aa, Escherichia coli
MTDKLTSLRQYTTVVADTGDIAAMKLYQPQDATTNPSLILNAAQIPEYRKLIDDAVAWAKQQSNDRAQQIVDATDKLAVNIGLEILKLVPGRISTEVDARLSYDTEASIAKAKRLIKLYNDAGISNDRILIKLASTWQGIRAAEQLEKEGINCNLTLLFSFAQARACAEAGVFLISPFVGRILDWYKANTDKKEYAPAEDPGVVSVSEIYQYYKEHGYETVVMGASFRNIGEILELAGCDRLTIAPALLKELAESEGAIERKLSYTGEVKARPARITESEFLWQHNQDPMAVDKLAEGIRKFAIDQEKLEKMIGDLL

SEQ ID NO: 18 - DNA, 1992 bp, Escherichia coli
  CDS (1)..(1992)
atgtcctcacgtaaagagcttgccaatgctattcgtgcgctgagcatggacgcagtacagaaagccaaatccggtcacccgggtgcccctatgggtatggctgacattgccgaagtcctgtggcgtgatttcctgaaacacaacccgcagaatccgtcctgggctgaccgtgaccgcttcgtgctgtccaacggccacggctccatgctgatctacagcctgctgcacctcaccggttacgatctgccgatggaagaactgaaaaacttccgtcagctgcactctaaaactccgggtcacccggaagtgggttacaccgctggtgtggaaaccaccaccggtccgctgggtcagggtattgccaacgcagtcggtatggcgattgcagaaaaaacgctggcggcgcagtttaaccgtccgggccacgacattgtcgaccactacacctacgccttcatgggcgacggctgcatgatggaaggcatctcccacgaagtttgctctctggcgggtacgctgaagctgggtaaactgattgcattctacgatgacaacggtatttctatcgatggtcacgttgaaggctggttcaccgacgacaccgcaatgcgtttcgaagcttacggctggcacgttattcgcgacatcgacggtcatgacgcggcatctatcaaacgcgcagtagaagaagcgcgcgcagtgactgacaaaccttccctgctgatgtgcaaaaccatcatcggtttcggttccccgaacaaagccggtacccacgactcccacggtgcgccgctgggcgacgctgaaattgccctgacccgcgaacaactgggctggaaatatgcgccgttcgaaatcccgtctgaaatctatgctcagtgggatgcgaaagaagcaggccaggcgaaagaatccgcatggaacgagaaattcgctgcttacgcgaaagcttatccgcaggaagccgctgaatttacccgccgtatgaaaggcgaaatgccgtctgacttcgacgctaaagcgaaagagttcatcgctaaactgcaggctaatccggcgaaaatcgccagccgtaaagcgtctcagaatgctatcgaagcgttcggtccgctgttgccggaattcctcggcggttctgctgacctggcgccgtctaacctgaccctgtggtctggttctaaagcaatcaacgaagatgctgcgggtaactacatccactacggtgttcgcgagttcggtatgaccgcgattgctaacggtatctccctgcacggtggcttcctgccgtacacctccaccttcctgatgttcgtggaatacgcacgtaacgccgtacgtatggctgcgctgatgaaacagcgtcaggtgatggtttacacccacgactccatcggtctgggcgaagacggcccgactcaccagccggttgagcaggtcgcttctctgcgcgtaaccccgaacatgtctacatggcgtccgtgtgaccaggttgaatccgcggtcgcgtggaaatacggtgttgagcgtcaggacggcccgaccgcactgatcctctcccgtcagaacctggcgcagcaggaacgaactgaagagcaactggcaaacatcgcgcgcggtggttatgtgctgaaagactgcgccggtcagccggaactgattttcatcgctaccggttcagaagttgaactggctgttgctgcctacgaaaaactgactgccgaaggcgtgaaagcgcgcgtggtgtccatgccgtctaccgacgcatttgacaagcaggatgctgcttaccgtgaatccgtactgccgaaagcggttactgcacgcgttgctgtagaagcgggtattgctgactactggtacaagtatgttggcctgaacggtgctatcgtcggtatgaccaccttcggtgaatctgctccggcagagctgctgtttgaagagttcggcttcactgttgataacgttgttgcgaaagcaaaagaactgctgtaa

SEQ ID NO: 19 - protein, 663 aa, Escherichia coli
MSSRKELANAIRALSMDAVQKAKSGHPGAPMGMADIAEVLWRDFLKHNPQNPSWADRDRFVLSNGHGSMLIYSLLHLTGYDLPMEELKNFRQLHSKTPGHPEVGYTAGVETTTGPLGQGIANAVGMAIAEKTLAAQFNRPGHDIVDHYTYAFMGDGCMMEGISHEVCSLAGTLKLGKLIAFYDDNGISIDGHVEGWFTDDTAMRFEAYGWHVIRDIDGHDAASIKRAVEEARAVTDKPSLLMCKTIIGFGSPNKAGTHDSHGAPLGDAEIALTREQLGWKYAPFEIPSEIYAQWDAKEAGQAKESAWNEKFAAYAKAYPQEAAEFTRRMKGEMPSDFDAKAKEFIAKLQANPAKIASRKASQNAIEAFGPLLPEFLGGSADLAPSNLTLWSGSKAINEDAAGNYIHYGVREFGMTAIANGISLHGGFLPYTSTFLMFVEYARNAVRMAALMKQRQVMVYTHDSIGLGEDGPTHQPVEQVASLRVTPNMSTWRPCDQVESAVAWKYGVERQDGPTALILSRQNLAQQERTEEQLANIARGGYVLKDCAGQPELIFIATGSEVELAVAAYEKLTAEGVKARVVSMPSTDAFDKQDAAYRESVLPKAVTARVAVEAGIADYWYKYVGLNGAIVGMTTFGESAPAELLFEEFGFTVDNVVAKAKELL

SEQ ID NO: 20 - DNA, 1080 bp, Escherichia coli
  CDS (1)..(1080)
atgtctaagatttttgatttcgtaaaacctggcgtaatcactggtgatgacgtacagaaagttttccaggtagcaaaagaaaacaacttcgcactgccagcagtaaactgcgtcggtactgactccatcaacgccgtactggaaaccgctgctaaagttaaagcgccggttatcgttcagttctccaacggtggtgcttcctttatcgctggtaaaggcgtgaaatctgacgttccgcagggtgctgctatcctgggcgcgatctctggtgcgcatcacgttcaccagatggctgaacattatggtgttccggttatcctgcacactgaccactgcgcgaagaaactgctgccgtggatcgacggtctgttggacgcgggtgaaaaacacttcgcagctaccggtaagccgctgttctcttctcacatgatcgacctgtctgaagaatctctgcaagagaacatcgaaatctgctctaaatacctggagcgcatgtccaaaatcggcatgactctggaaatcgaactgggttgcaccggtggtgaagaagacggcgtggacaacagccacatggacgcttctgcactgtacacccagccggaagacgttgattacgcatacaccgaactgagcaaaatcagcccgcgtttcaccatcgcagcgtccttcggtaacgtacacggtgtttacaagccgggtaacgtggttctgactccgaccatcctgcgtgattctcaggaatatgtttccaagaaacacaacctgccgcacaacagcctgaacttcgtattccacggtggttccggttctactgctcaggaaatcaaagactccgtaagctacggcgtagtaaaaatgaacatcgataccgatacccaatgggcaacctgggaaggcgttctgaactactacaaagcgaacgaagcttatctgcagggtcagctgggtaacccgaaaggcgaagatcagccgaacaagaaatactacgatccgcgcgtatggctgcgtgccggtcagacttcgatgatcgctcgtctggagaaagcattccaggaactgaacgcgatcgacgttctgtaa

SEQ ID NO: 21 - protein, 359 aa, Escherichia coli
MSKIFDFVKPGVITGDDVQKVFQVAKENNFALPAVNCVGTDSINAVLETAAKVKAPVIVQFSNGGASFIAGKGVKSDVPQGAAILGAISGAHHVHQMAEHYGVPVILHTDHCAKKLLPWIDGLLDAGEKHFAATGKPLFSSHMIDLSEESLQENIEICSKYLERMSKIGMTLEIELGCTGGEEDGVDNSHMDASALYTQPEDVDYAYTELSKISPRFTIAASFGNVHGVYKPGNVVLTPTILRDSQEYVSKKHNLPHNSLNFVFHGGSGSTAQEIKDSVSYGVVKMNIDTDTQWATWEGVLNYYKANEAYLQGQLGNPKGEDQPNKKYYDPRVWLRAGQTSMIARLEKAFQELNAIDVL

SEQ ID NO: 22 - protein, 394 aa, Geobacillus stearothermophilus
MNKKTIRDVDVRGKRVFCRVDFNVPMEQGAITDDTRIRAALPTIRYLIEHGAKVILASHLGRPKGKVVEELRLDAVAKRLGELLERPVAKTNEAVGDEVKAAVDRLNEGDVLLLENVRFYPGEEKNDPELAKAFAELADLYVNDAFGAAHRAHASTEGIAHYLPAVAGFLMEKELEVLGKALSNPDRPFTAIIGGAKVKDKIGVIDNLLEKVDNLIIGGGLAYTFVKALGHDVGKSLLEEDKIELAKSFMEKAKEKGVRFYMPVDVVVADRFANDANTKVVPIDAIPADWSALDIGPKTRELYRDVIRESKLVVWNGPMGVFEMDAFAHGTKAIAEALAEALDTYSVIGGGDSAAAVEKFGLADKMDHISTGGGASLEFMEGKQLPGVVALEDK

SEQ ID NO: 23 - DNA, 20 bp, Artificial Sequence
  rpiA knockout forward primer
cgccttctaccagcagaaac

SEQ ID NO: 24 - DNA, 20 bp, Artificial Sequence
  rpiA knockout reverse primer
cccagaccgttgtatgcttt

SEQ ID NO: 25 - DNA, 20 bp, Artificial Sequence
  rpiB knockout forward primer
ggaagcgctgaatcaaactc

SEQ ID NO: 26 - DNA, 20 bp, Artificial Sequence
  rpiB knockout reverse primer
gctcttcatcctccagttgc

SEQ ID NO: 27 - DNA, 20 bp, Artificial Sequence
  nupG knockin forward primer
atatgccatttgccacacca

SEQ ID NO: 28 - DNA, 20 bp, Artificial Sequence
  nupG knockin reverse primer
cttatattcgcggtgacgtg

SEQ ID NO: 29 - DNA, 24 bp, Artificial Sequence
  SS3 knockin forward primer
tgttaattagcgggcaattgtacc

SEQ ID NO: 30 - DNA, 24 bp, Artificial Sequence
  SS3 knockin reverse primer
gatacctacagcgcagaaaaacaa

SEQ ID NO: 31 - DNA, 23 bp, Artificial Sequence
  gapA knockout/gapC knockin forward primer
tgcttcgatattatggcgggctt

SEQ ID NO: 32 - DNA, 23 bp, Artificial Sequence
  gapA knockout/gapC knockin reverse primer
gccagatgtgcaggtttctcttt

SEQ ID NO: 33 - DNA, 23 bp, Artificial Sequence
  pfkA knckout forward primer
atcaatcttatggacggctggtc

SEQ ID NO: 34 - DNA, 23 bp, Artificial Sequence
  pfkA knockout reverse primer
tgctgatctgatcgaacgtaccg

SEQ ID NO: 35 - DNA, 20 bp, Artificial Sequence
  frmA frameshift validation forward primer
tattttgccagccgccaaag

SEQ ID NO: 36 - DNA, 20 bp, Artificial Sequence
  frmA frameshift validation reverse primer
cgaaatgactgctacagccg

SEQ ID NO: 37 - DNA, 18 bp, Artificial Sequence
  pgi deletion validation forward primer
gaagtcaacgcggtgctg

SEQ ID NO: 38 - DNA, 18 bp, Artificial Sequence
  pgi deletion validation reverse primer
ccctggtggatcagctgg

SEQ ID NO: 39 - DNA, 25 bp, Artificial Sequence
  pFC139 plasmid variation validation forward primer
atcctactgcttttttcaattcatc

SEQ ID NO: 40 - DNA, 20 bp, Artificial Sequence
  pFC139 plasmid variation validation reverse primer
caagggtgaacactatccca

SEQ ID NO: 41 - DNA, 963 bp, Escherichia coli
  CDS (1)..(963)
atgattaagaaaatcggtgtgttgacaagcggcggtgatgcgccaggcatgaacgccgcaattcgcggggttgttcgttctgcgctgacagaaggtctggaagtaatgggtatttatgacggctatctgggtctgtatgaagaccgtatggtacagctagaccgttacagcgtgtctgacatgatcaaccgtggcggtacgttcctcggttctgcgcgtttcccggaattccgcgacgagaacatccgcgccgtggctatcgaaaacctgaaaaaacgtggtatcgacgcgctggtggttatcggcggtgacggttcctacatgggtgcaatgcgtctgaccgaaatgggcttcccgtgcatcggtctgccgggcactatcgacaacgacatcaaaggcactgactacactatcggtttcttcactgcgctgagcaccgttgtagaagcgatcgaccgtctgcgtgacacctcttcttctcaccagcgtatttccgtggtggaagtgatgggccgttattgtggagatctgacgttggctgcggccattgccggtggctgtgaattcgttgtggttccggaagttgaattcagccgtgaagacctggtaaacgaaatcaaagcgggtatcgcgaaaggtaaaaaacacgcgatcgtggcgattaccgaacatatgtgtgatgttgacgaactggcgcatttcatcgagaaagaaaccggtcgtgaaacccgcgcaactgtgctgggccacatccagcgcggtggttctccggtgccttacgaccgtattctggcttcccgtatgggcgcttacgctatcgatctgctgctggcaggttacggcggtcgttgtgtaggtatccagaacgaacagctggttcaccacgacatcatcgacgctatcgaaaacatgaagcgtccgttcaaaggtgactggctggactgcgcgaaaaaactgtattaa

SEQ ID NO: 42 - protein, 320 aa, Escherichia coli
MIKKIGVLTSGGDAPGMNAAIRGVVRSALTEGLEVMGIYDGYLGLYEDRMVQLDRYSVSDMINRGGTFLGSARFPEFRDENIRAVAIENLKKRGIDALVVIGGDGSYMGAMRLTEMGFPCIGLPGTIDNDIKGTDYTIGFFTALSTVVEAIDRLRDTSSSHQRISVVEVMGRYCGDLTLAAAIAGGCEFVVVPEVEFSREDLVNEIKAGIAKGKKHAIVAITEHMCDVDELAHFIEKETGRETRATVLGHIQRGGSPVPYDRILASRMGAYAIDLLLAGYGGRCVGIQNEQLVHHDIIDAIENMKRPFKGDWLDCAKKLY

SEQ ID NO: 43 - DNA, 930 bp, Escherichia coli
  CDS (1)..(930)
atggtacgtatctatacgttgacacttgcgccctctctcgatagcgcaacaattaccccgcaaatttatcccgaaggaaaactgcgctgtaccgcaccggtgttcgaacccgggggcggcggcatcaacgtcgcccgcgccattgcccatcttggaggcagtgccacagcgatcttcccggcgggtggcgcgaccggcgaacacctggtttcactgttggcggatgaaaatgtccccgtcgctactgtagaagccaaagactggacccggcagaatttacacgtacatgtggaagcaagcggtgagcagtatcgttttgttatgccaggcgcggcattaaatgaagatgagtttcgccagcttgaagagcaagttctggaaattgaatccggggccatcctggtcataagcggaagcctgccgccaggtgtgaagctggaaaaattaacccaactgatttccgctgcgcaaaaacaagggatccgctgcatcgtcgacagttctggcgaagcgttaagtgcagcactggcaattggtaacatcgagttggttaagcctaaccaaaaagaactcagtgcgctggtgaatcgcgaactcacccagccggacgatgtccgcaaagccgcgcaggaaatcgttaatagcggcaaggccaaacgggttgtcgtttccctgggtccacaaggagcgctgggtgttgatagtgaaaactgtattcaggtggtgccaccaccggtgaaaagccagagtaccgttggcgctggtgacagcatggtcggcgcgatgacactgaaactggcagaaaatgcctctcttgaagagatggttcgttttggcgtagctgcggggagtgcagccacactcaatcagggaacacgtctgtgctcccatgacgatacgcaaaaaatttacgcttacctttcccgctaa

SEQ ID NO: 44 - protein, 309 aa, Escherichia coli
MVRIYTLTLAPSLDSATITPQIYPEGKLRCTAPVFEPGGGGINVARAIAHLGGSATAIFPAGGATGEHLVSLLADENVPVATVEAKDWTRQNLHVHVEASGEQYRFVMPGAALNEDEFRQLEEQVLEIESGAILVISGSLPPGVKLEKLTQLISAAQKQGIRCIVDSSGEALSAALAIGNIELVKPNQKELSALVNRELTQPDDVRKAAQEIVNSGKAKRVVVSLGPQGALGVDSENCIQVVPPPVKSQSTVGAGDSMVGAMTLKLAENASLEEMVRFGVAAGSAATLNQGTRLCSHDDTQKIYAYLSR

SEQ ID NO: 45 - DNA, 35 bp, Artificial Sequence
  RBS sequence
gactaaaaacattcggaggcttaagcagtcatcgt